SEQUENCE LISTING (3 sequences)

SEQ ID NO: 1 - DNA, 20 bp, Artificial Sequence
  Description of Artificial Sequence Synthetic primer
aagaaaaaccccgcctgtaa

SEQ ID NO: 2 - DNA, 23 bp, Artificial Sequence
  Description of Artificial Sequence Synthetic primer
cctgttctgactactgcctctcc

SEQ ID NO: 3 - DNA, 27 bp, Artificial Sequence
  Description of Artificial Sequence Synthetic probe
cctgatgtgatgttctccatgttcagc

(19) United States
(12) Patent Application Publication
(10) Pub. No.: US 2022/0213049 A1
(43) Pub. Date: Jul. 7, 2022

(54) SUBSTITUTED CHROMEN-4-ONE FOR THE TREATMENT AND PROPHYLAXIS OF HEPATITIS B VIRUS INFECTION

(71) Applicant: Hoffmann-La Roche Inc., Little Falls, NJ (US)

(72) Inventors: Dongdong CHEN, Shanghai (CN); Song FENG, Shanghai (CN); Chungen LIANG, Shanghai (CN); Kun MIAO, Shanghai (CN); Xuefei TAN, Shanghai (CN); Hong SHEN, Shanghai (CN)

(73) Assignee: Hoffmann-La Roche Inc., Little Falls, NJ (US)

(21) Appl. No.: 17/423,073

(22) PCT Filed: Jan. 16, 2020
(86) PCT No.: PCT/EP2020/050953

§ 371 Date: Jul. 14, 2021

(30) Foreign Application Priority Data
Jan. 17, 2019 (CN) PCT/CN2019/072117

(51) Int. Cl. C07D 311/30; A61P 31/20

(52) U.S. Cl. CPC C07D 311/30; A61P 31/20

(57) ABSTRACT

The present invention provides novel compounds having the general formula: (I) wherein R¹ to R¹⁰, Gᵢ, G₂ and m are as described herein, compositions including the impounds and methods of using the compounds for the treatment of hepatitis B.

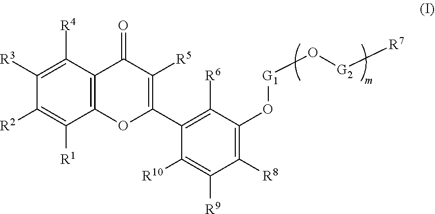

The present invention relates to organic compounds useful for therapy and/or prophylaxis of HBV infection in a mammal, and in particular to cccDNA (covalently closed circular DNA) inhibitors useful for treating HBV infection.

### FIELD OF THE INVENTION

The present invention relates to substituted chromen-4-one having pharmaceutical activity, their manufacture, pharmaceutical compositions containing them and their potential use as medicaments.

The present invention relates to compounds of formula (I)

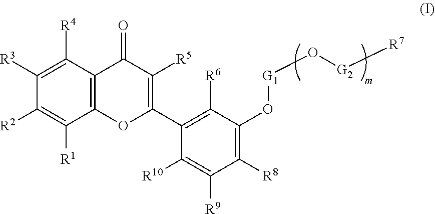

wherein R¹ to R¹⁰, G₁, G₂ and m are as described below, or a pharmaceutically acceptable salt thereof.

Hepatitis B virus (HBV) infection is one of the most prevalent viral infections and is a leading cause of chronic hepatitis. It is estimated that worldwide, around 2 billion people have evidence of past or present infection with HBV. Over 250 million individuals are currently chronically infected with HBV and are therefore at high risk to develop liver fibrosis, cirrhosis and hepatocellular carcinoma (HCC). There are data to indicate ˜800,000 deaths per year are directly linked to HBV infection (Lozano, R. et al., Lancet (2012), 380 (9859), 2095-2128; Goldstein, S. T. et al., Int J Epidemiol (2005), 34 (6), 1329-1339).

Many countries in the world administer hepatitis B vaccine starting at birth or in early childhood, which has greatly reduced the incidence and prevalence of hepatitis B in most endemic regions over the past few decades. However, the vaccine has no impact on people who were infected before the widely use of the vaccine in developing end-stage liver disease or HCC (Chen, D. S., J Hepatol (2009), 50 (4), 805-816). Vaccination at birth of infants born to HBV positive mothers is usually not sufficient for protecting vertical transmission and combination with hepatitis B immune globulin is needed (Li, X. M. et al., World J Gastroenterol (2003), 9 (7), 1501-1503).

Currently FDA-approved treatments for chronic hepatitis B include two type 1 interferons (IFN) which are IFNalfa-2b and pegylated IFN alfa-2a and six nucleos(t)ide analogues (NAs) which are lamivudine (3TC), tenofovir disoproxil fumarate (TDF), adefovir (ADV), telbivudine (LdT), entecavir (ETV), and vemlidy (tenofovir alafenamide (TAF)). IFN treatment is finite, but it is known to have severe side effects, and only a small percentage of patients showed a sustained virological response, measured as loss of hepatitis B surface antigen (HBsAg). NAs are inhibitors of the HBV reverse transcriptase, profoundly reduce the viral load in vast majority of treated patients, and lead to improvement of liver function and reduced incidence of liver failure and hepatocellular carcinoma. However, the treatment of NAs is infinite (Ahmed, M. et al., Drug Discov Today (2015), 20 (5), 548-561; Zoulim, F. and Locarnini, S., Gastroenterology (2009), 137 (5), 1593-1608 e1591-1592).

HBV chronic infection is caused by persistence of covalently closed circular (ccc)DNA, which exists as an episomal form in hepatocyte nuclei. cccDNA serves as the template for viral RNA transcription and subsequent viral DNA generation. Only a few copies of cccDNA per liver cell can establish or re-initiate viral replication. Therefore, a complete cure of chronic hepatitis B will require elimination of cccDNA or permanently silencing of cccDNA. However, cccDNA is intrinsically very stable and currently available therapeutics could not eliminate cccDNA or permanently silence cccDNA (Nassal, M., Gut (2015), 64 (12), 1972-1984; Gish, R. G. et al., Antiviral Res (2015), 121, 47-58; Levrero, M. et al., J Hepatol (2009), 51 (3), 581-592.). The current SoC could not eliminate the cccDNA which are already present in the infected cells. There is an urgent need to discover and develop new anti-HBV reagents to eliminate or permanently silence cccDNA, the source of chronicity (Ahmed, M. et al., Drug Discov Today (2015), 20 (5), 548-561; Nassal, M., Gut (2015), 64 (12), 1972-1984).

### SUMMARY OF THE INVENTION

Objects of the present invention are compounds of formula (I), their manufacture, medicaments based on a compound in accordance with the invention and their production as well as the use of compounds of formula (I) as cccDNA inhibitors and for the treatment or prophylaxis of HBV infection. The compounds of formula (I) show superior anti-HBV activity. In addition, the compounds of formula (I) also show good PK profiles.

The present invention relates to a compound of formula (I)

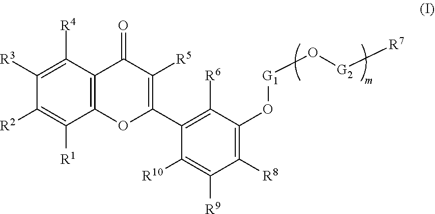

wherein
R¹ is halogen;
R² is selected from H, OH, halogen, C₁₋₆alkyl, haloC₁₋₆alkyl and C₁₋₆alkoxy;
R³ is selected from H, OH, halogen, C₁₋₆alkyl, haloC₁₋₆alkyl and C₁₋₆alkoxy;
R⁴ is selected from H, OH, halogen, C₁₋₆alkyl, haloC₁₋₆alkyl and C₁₋₆alkoxy;
R⁵ is selected from H, OH, halogen, C₁₋₆alkyl, haloC₁₋₆alkyl and C₁₋₆alkoxy;
R⁶ is selected from H, OH, halogen, C₁₋₆alkyl, haloC₁₋₆alkyl and C₁₋₆alkoxy;
R⁷ is selected from carboxy, C₁₋₆alkoxycarbonyl, carboxycarbonylamino, C₃₋₇ cycloalkylsulfonylaminocarbonyl and C₁₋₆alkoxycarbonylcarbonylamino;
R⁸ is selected from H, OH, halogen, C₁₋₆alkyl, haloC₁₋₆alkyl and C₁₋₆alkoxy;
R⁹ is selected from H, OH, halogen, C₁₋₆alkyl, haloC₁₋₆alkyl and C₁₋₆alkoxy;
R¹⁰ is selected from H, OH, halogen, C₁₋₆alkyl, haloC₁₋₆alkyl, C₁₋₆alkoxy and haloC₁₋₆alkoxy;
G₁ is selected from C₁₋₆alkyl and C₃₋₇cycloalkyl;
G₂ is selected from C₁₋₆alkyl and C₃₋₇cycloalkyl;
m is selected from 0 and 1;
or a pharmaceutically acceptable salt thereof.

### DETAILED DESCRIPTION OF THE INVENTION

### Definitions

As used herein, the term “C₁₋₆alkyl” alone or in combination signifies a saturated, linear- or branched chain alkyl group containing 1 to 6, particularly 1 to 4 carbon atoms, for example methyl, ethyl, propyl, isopropyl, butyl, isobutyl, tert-butyl and the like. Particular “C₁₋₆alkyl” groups are methyl, ethyl, propyl, isopropyl, isobutyl and tert-butyl. Most particular “C₁₋₆alkyl” group is methyl.

The term “C₁₋₆alkoxy” alone or in combination signifies a group C₁₋₆alkyl-O—, wherein the “C₁₋₆alkyl” is as defined above; for example, methoxy, ethoxy, propoxy, iso-propoxy, n-butoxy, iso-butoxy, 2-butoxy, tert-butoxy, pentoxy, hexyloxy and the like. Particular “C₁₋₆alkoxy” groups are methoxy and ethoxy.

The term “C₃₋₇cycloalkyl” denotes to a saturated carbon ring containing from 3 to 7 carbon atoms, particularly from 3 to 6 carbon atoms, for example, cyclopropyl, cyclobutyl, cyclopentyl, cyclohexyl, cycloheptyl and the like. Particular “C₃₋₇cycloalkyl” groups are cyclopropyl and cyclobutyl.

The term “halogen” and “halo” are used interchangeably herein and denote fluoro, chloro, bromo, or iodo.

The term “haloC₁₋₆alkyl” denotes an alkyl group wherein at least one of the hydrogen atoms of the alkyl group is replaced by same or different halogen atoms, particularly fluoro atoms. Examples of haloC₁₋₆alkyl include monochloro-, difluoro- or trifluoro-methyl, -ethyl or -propyl, for example difluoromethyl and trifluoromethyl.

The term “haloC₁₋₆alkoxy” denotes a C₁₋₆alkoxy group wherein at least one of the hydrogen atoms of the C₁₋₆alkoxy group is replaced by same or different halogen atoms, particularly fluoro atoms. Examples of haloC₁₋₆alkoxy include monofluoro-, difluoro- or trifluoro-methoxy, -ethoxy or -propoxy, for example difluoromethoxy and trifluoromethoxy.

The term “carbonyl” alone or in combination refers to the group —C(O)—.

The term “sulfonyl” alone or in combination refers to the group —S(O)₂—.

The compounds according to the present invention may exist in the form of their pharmaceutically acceptable salts. The term “pharmaceutically acceptable salt” refers to conventional acid-addition salts or base-addition salts that retain the biological effectiveness and properties of the compounds of formula (I) and are formed from suitable non-toxic organic or inorganic acids or organic or inorganic bases. Acid-addition salts include for example those derived from inorganic acids such as hydrochloric acid, hydrobromic acid, hydroiodic acid, sulfuric acid, sulfamic acid, phosphoric acid and nitric acid, and those derived from organic acids such asp-toluenesulfonic acid, salicylic acid, methanesulfonic acid, oxalic acid, succinic acid, citric acid, malic acid, lactic acid, fumaric acid, and the like. Base-addition salts include those derived from ammonium, potassium, sodium and, quaternary ammonium hydroxides, such as for example, tetramethyl ammonium hydroxide. The chemical modification of a pharmaceutical compound into a salt is a technique well known to pharmaceutical chemists in order to obtain improved physical and chemical stability, hygroscopicity, flowability and solubility of compounds. It is for example described in Bastin R. J., et al., Organic Process Research & Development 2000, 4, 427-435. Particular are the sodium salts of the compounds of formula (I).

Compounds of the general formula (I) which contain one or several chiral centers can either be present as racemates, diastereomeric mixtures, or optically active single isomers. The racemates can be separated according to known methods into the enantiomers. Particularly, diastereomeric salts which can be separated by crystallization are formed from the racemic mixtures by reaction with an optically active acid such as e.g. D- or L-tartaric acid, mandelic acid, malic acid, lactic acid or camphorsulfonic acid.

### HBV Inhibitors

The present invention provides (i) a compound having the general formula (I):

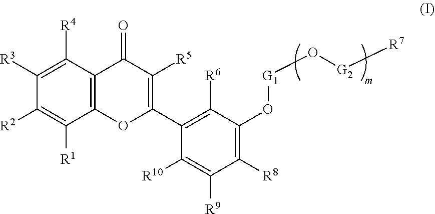

wherein
R¹ is halogen;
R² is selected from H, OH, halogen, C₁₋₆alkyl, haloC₁₋₆alkyl and C₁₋₆alkoxy;
R³ is selected from H, OH, halogen, C₁₋₆alkyl, haloC₁₋₆alkyl and C₁₋₆alkoxy;
R⁴ is selected from H, OH, halogen, C₁₋₆alkyl, haloC₁₋₆alkyl and C₁₋₆alkoxy;
R⁵ is selected from H, OH, halogen, C₁₋₆alkyl, haloC₁₋₆alkyl and C₁₋₆alkoxy;
R⁶ is selected from H, OH, halogen, C₁₋₆alkyl, haloC₁₋₆alkyl and C₁₋₆alkoxy;
R⁷ is selected from carboxy, C₁₋₆alkoxycarbonyl, carboxycarbonylamino, C₃₋₇cycloalkylsulfonylaminocarbonyl and C₁₋₆alkoxycarbonylcarbonylamino;
R⁸ is selected from H, OH, halogen, C₁₋₆alkyl, haloC₁₋₆alkyl and C₁₋₆alkoxy;
R⁹ is selected from H, OH, halogen, C₁₋₆alkyl, haloC₁₋₆alkyl and C₁₋₆alkoxy;
R¹⁰ is selected from H, OH, halogen, C₁₋₆alkyl, haloC₁₋₆alkyl, C₁₋₆alkoxy and haloC₁₋₆alkoxy;
G₁ is selected from C₁₋₆alkyl and C₃₋₇cycloalkyl;
G₂ is selected from C₁₋₆alkyl and C₃₋₇cycloalkyl;
m is selected from 0 and 1;
or a pharmaceutically acceptable salt thereof.

A further embodiment of the present invention is (ii) a compound of formula (I) according to (i), wherein
R¹ is halogen;
R² is H;
R³ is H;
R⁴ is H;
R⁵ is H;
R⁶ is selected from H and C₁₋₆alkoxy;
R⁷ is selected from carboxy, carboxycarbonylamino and C₃₋₇cycloalkylsulfonylaminocarbonyl;
R⁸ is selected from H, halogen, C₁₋₆alkyl, haloC₁₋₆alkyl and C₁₋₆alkoxy;
R⁹ is selected from H and C₁₋₆alkoxy;
R¹⁰ is selected from H, halogen, C₁₋₆alkoxy and haloC₁₋₆alkoxy;
G₁ is selected from C₁₋₆alkyl and C₃₋₇cycloalkyl;
G₂ is selected from C₁₋₆alkyl and C₃₋₇cycloalkyl;
m is selected from 0 and 1;
or a pharmaceutically acceptable salt thereof.

A further embodiment of the present invention is (iii) a compound of formula (I) according to (i), wherein
R¹ is Cl;
R² is H;
R³ is H;
R⁴ is H;
R⁵ is H;
R⁶ is selected from H and methoxy;
R⁷ is selected from carboxy, carboxycarbonylamino and cyclopropylsulfonylaminocarbonyl;
R⁸ is selected from H, Cl, Br, methyl, CF₃ and methoxy;
R⁹ is selected from H and methoxy;
R¹⁰ is selected from H, F, Cl, Br, methoxy, ethoxy, difluoromethoxy and trifluoromethoxy;
G₁ is selected from methyl, ethyl, propyl and cyclobutyl;
G₂ is selected from methyl and cyclobutyl;
m is selected from 0 and 1;
or a pharmaceutically acceptable salt thereof.

A further embodiment of the present invention is (iv) a compound of formula (I) according to (i), or a pharmaceutically acceptable salt thereof, wherein R⁷ is selected from carboxy and C₃₋₇ cycloalkylsulfonylaminocarbonyl.

A further embodiment of the present invention is (v) a compound of formula (I) according to (i), or a pharmaceutically acceptable salt thereof, wherein R⁷ is selected from carboxy and cyclopropylsulfonylaminocarbonyl.

A further embodiment of the present invention is (vi) a compound of formula (I) according to (i), or a pharmaceutically acceptable salt thereof, wherein R⁹ is H.

A further embodiment of the present invention is (vii) a compound of formula (I) according to (i), or a pharmaceutically acceptable salt thereof, wherein R¹⁰ is selected from H, halogen and C₁₋₆alkoxy.

A further embodiment of the present invention is (viii) a compound of formula (I) according to (i), or a pharmaceutically acceptable salt thereof, wherein R¹⁰ is selected from H, F, methoxy and ethoxy.

A further embodiment of the present invention is (ix) a compound of formula (II) according to (i), or a pharmaceutically acceptable salt thereof,

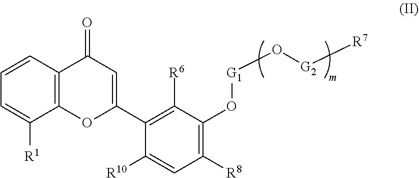

wherein
R¹ is halogen;
R⁶ is selected from H and C₁₋₆alkoxy;
R⁷ is selected from carboxy and C₃₋₇cycloalkylsulfonylaminocarbonyl;
R⁸ is selected from H, halogen, C₁₋₆alkyl, haloC₁₋₆alkyl and C₁₋₆alkoxy;
R¹⁰ is selected from H, halogen and C₁₋₆alkoxy;
G₁ is selected from C₁₋₆alkyl and C₃₋₇cycloalkyl;
G₂ is selected from C₁₋₆alkyl and C₃₋₇cycloalkyl;
m is selected from 0 and 1.

A further embodiment of the present invention is (x) a compound of formula (II) according to (i), or a pharmaceutically acceptable salt thereof, wherein
R¹ is Cl;
R⁶ is selected from H and methoxy;
R⁷ is selected from carboxy and cyclopropylsulfonylaminocarbonyl;
R⁸ is selected from H, Cl, Br, methyl, CF₃ and methoxy;
R¹⁰ is selected from H, F, methoxy and ethoxy;
G₁ is selected from methyl, ethyl, propyl and cyclobutyl;
G₂ is selected from methyl and cyclobutyl;
m is selected from 0 and 1.

In another embodiment (xi) of the present invention, particular compounds of the present invention are selected from:
cis-3-[2-[2-bromo-5-(8-chloro-4-oxo-chromen-2-yl)-4-methoxy-phenoxy]ethoxy]cyclobutanecarboxylic acid;
3-[2-bromo-5-(8-chloro-4-oxo-chromen-2-yl)-4-methoxy-phenoxy]propanoic acid;
3-[2-bromo-5-(8-chloro-4-oxo-chromen-2-yl)-4-methoxy-phenoxy]-N-cyclopropylsulfonyl-propanamide;
2-[2-bromo-5-(8-chloro-4-oxo-chromen-2-yl)-4-methoxy-phenoxy]acetic acid;
3-[2-bromo-5-(8-chloro-4-oxo-chromen-2-yl)-4-methoxy-phenoxy]cyclobutanecarboxylic acid;
2-[3-[2-bromo-5-(8-chloro-4-oxo-chromen-2-yl)-4-methoxy-phenoxy]propoxy]acetic acid;
3-[2-chloro-5-(8-chloro-4-oxo-chromen-2-yl)-4-methoxy-phenoxy]cyclobutanecarboxylic acid;
cis-3-[2-[2-chloro-5-(8-chloro-4-oxo-chromen-2-yl)-4-methoxy-phenoxy]ethoxy]cyclobutanecarboxylic acid;
3-[2-[5-(8-chloro-4-oxo-chromen-2-yl)-4-methoxy-2-methyl-phenoxy]ethoxy]cyclobutanecarboxylic acid;
3-[5-(8-chloro-4-oxo-chromen-2-yl)-4-methoxy-2-methyl-phenoxy]propanoic acid;
2-[3-[5-(8-chloro-4-oxo-chromen-2-yl)-4-methoxy-2-methyl-phenoxy]propoxy]acetic acid;
3-[5-(8-chloro-4-oxo-chromen-2-yl)-2,4-dimethoxy-phenoxy]cyclobutanecarboxylic acid;
3-[2-[5-(8-chloro-4-oxo-chromen-2-yl)-2,4-dimethoxy-phenoxy]ethoxy]cyclobutanecarboxylic acid;
2-[5-(8-chloro-4-oxo-chromen-2-yl)-2-methyl-phenoxy]acetic acid;
cis-3-[5-(8-chloro-4-oxo-chromen-2-yl)-2-methyl-phenoxy]cyclobutanecarboxylic acid;
trans-3-[5-(8-chloro-4-oxo-chromen-2-yl)-2-methyl-phenoxy]cyclobutanecarboxylic acid;
3-[5-(8-chloro-4-oxo-chromen-2-yl)-2-methoxy-phenoxy]cyclobutanecarboxylic acid;
3-[2-[5-(8-chloro-4-oxo-chromen-2-yl)-2-methoxy-phenoxy]ethoxy]cyclobutanecarboxylic acid;
3-[5-(8-chloro-4-oxo-chromen-2-yl)-2-methoxy-phenoxy]propanoic acid;
cis-3-[5-(8-chloro-4-oxo-chromen-2-yl)-2-(trifluoromethyl)phenoxy]cyclobutanecarboxylic acid;
trans-3-[5-(8-chloro-4-oxo-chromen-2-yl)-2-(trifluoromethyl)phenoxy]cyclobutanecarboxylic acid;
2-[5-(8-chloro-4-oxo-chromen-2-yl)-2-(trifluoromethyl)phenoxy]acetic acid;
cis-3-[5-(8-chloro-4-oxo-chromen-2-yl)-2,3-dimethoxy-phenoxy]cyclobutanecarboxylic acid;
trans-3-[2-[5-(8-chloro-4-oxo-chromen-2-yl)-2,3-dimethoxy-phenoxy]ethoxy]cyclobutanecarboxylic acid;
2-[5-(8-chloro-4-oxo-chromen-2-yl)-2,3-dimethoxy-phenoxy]acetic acid;
trans-3-[4-bromo-5-(8-chloro-4-oxo-chromen-2-yl)-2-methoxy-phenoxy]cyclobutanecarboxylic acid;
trans-3-[2-[4-bromo-5-(8-chloro-4-oxo-chromen-2-yl)-2-methoxy-phenoxy]ethoxy]cyclobutanecarboxylic acid;
3-[3-(8-chloro-4-oxo-chromen-2-yl)-2-methoxy-phenoxy]cyclobutanecarboxylic acid;
cis-3-[2-[3-(8-chloro-4-oxo-chromen-2-yl)-2-methoxy-phenoxy]ethoxy]cyclobutanecarboxylic acid;
3-[3-(8-chloro-4-oxo-chromen-2-yl)-4-methoxy-phenoxy]cyclobutanecarboxylic acid;
cis-3-[2-[3-(8-chloro-4-oxo-chromen-2-yl)-4-methoxy-phenoxy]ethoxy]cyclobutanecarboxylic acid;
2-[3-(8-chloro-4-oxo-chromen-2-yl)-4-methoxy-phenoxy]acetic acid;
3-[3-(8-chloro-4-oxo-chromen-2-yl)-4-methoxy-phenoxy]propanoic acid;
cis-3-[2-[6-bromo-3-(8-chloro-4-oxo-chromen-2-yl)-2-methoxy-phenoxy]ethoxy]cyclobutanecarboxylic acid;
3-[6-bromo-3-(8-chloro-4-oxo-chromen-2-yl)-2-methoxy-phenoxy]cyclobutanecarboxylic acid;
3-[3-(8-chloro-4-oxo-chromen-2-yl)-2-methoxy-6-methyl-phenoxy]cyclobutanecarboxylic acid;
2-[6-chloro-3-(8-chloro-4-oxo-chromen-2-yl)-2-methoxy-phenoxy]acetic acid;
2-[3-(8-chloro-4-oxo-chromen-2-yl)-2,6-dimethoxy-phenoxy]acetic acid;
3-[2-bromo-5-(8-chloro-4-oxo-chromen-2-yl)-4-ethoxy-phenoxy]propanoic acid;
3-[2-bromo-5-(8-chloro-4-oxo-chromen-2-yl)-4-(difluoromethoxy)phenoxy]propanoic acid;
3-[2-bromo-5-(8-chloro-4-oxo-chromen-2-yl)-4-fluoro-phenoxy]propanoic acid;
3-[2-bromo-5-(8-chloro-4-oxo-chromen-2-yl)-4-(trifluoromethoxy)phenoxy]propanoic acid;
3-[2-bromo-4-chloro-5-(8-chloro-4-oxo-chromen-2-yl)phenoxy]propanoic acid;
2-[3-[6-bromo-3-(8-chloro-4-oxo-chromen-2-yl)-2-methoxy-phenoxy]propylamino]-2-oxo-acetic acid;
2-[2-[2-bromo-5-(8-chloro-4-oxo-chromen-2-yl)-4-methoxy-phenoxy]ethylamino]-2-oxo-acetic acid; and
2-[3-[2-bromo-5-(8-chloro-4-oxo-chromen-2-yl)-4-methoxy-phenoxy]propylamino]-2-oxo-acetic acid;
or a pharmaceutically acceptable salt thereof.

In another embodiment (xii) of the present invention, particular compounds of the present invention are selected from:
3-[2-bromo-5-(8-chloro-4-oxo-chromen-2-yl)-4-methoxy-phenoxy]propanoic acid;
3-[2-bromo-5-(8-chloro-4-oxo-chromen-2-yl)-4-methoxy-phenoxy]-N-cyclopropylsulfonyl-propanamide;
2-[2-bromo-5-(8-chloro-4-oxo-chromen-2-yl)-4-methoxy-phenoxy]acetic acid;
2-[3-[2-bromo-5-(8-chloro-4-oxo-chromen-2-yl)-4-methoxy-phenoxy]propoxy]acetic acid;
cis-3-[2-[2-chloro-5-(8-chloro-4-oxo-chromen-2-yl)-4-methoxy-phenoxy]ethoxy]cyclobutanecarboxylic acid;
3-[5-(8-chloro-4-oxo-chromen-2-yl)-2,4-dimethoxy-phenoxy]cyclobutanecarboxylic acid;
3-[2-[5-(8-chloro-4-oxo-chromen-2-yl)-2,4-dimethoxy-phenoxy]ethoxy]cyclobutanecarboxylic acid;
cis-3-[5-(8-chloro-4-oxo-chromen-2-yl)-2-methyl-phenoxy]cyclobutanecarboxylic acid;
2-[5-(8-chloro-4-oxo-chromen-2-yl)-2-(trifluoromethyl)phenoxy]acetic acid;
3-[3-(8-chloro-4-oxo-chromen-2-yl)-2-methoxy-phenoxy]cyclobutanecarboxylic acid;
cis-3-[2-[6-bromo-3-(8-chloro-4-oxo-chromen-2-yl)-2-methoxy-phenoxy]ethoxy]cyclobutanecarboxylic acid;
3-[6-bromo-3-(8-chloro-4-oxo-chromen-2-yl)-2-methoxy-phenoxy]cyclobutanecarboxylic acid;
2-[3-(8-chloro-4-oxo-chromen-2-yl)-2,6-dimethoxy-phenoxy]acetic acid;
3-[2-bromo-5-(8-chloro-4-oxo-chromen-2-yl)-4-ethoxy-phenoxy]propanoic acid; and
3-[2-bromo-5-(8-chloro-4-oxo-chromen-2-yl)-4-fluoro-phenoxy]propanoic acid;
or a pharmaceutically acceptable salt thereof.

### Synthesis

The compounds of the present invention can be prepared by any conventional means. Suitable processes for synthesizing these compounds as well as their starting materials are provided in the schemes below and in the examples. All substituents, in particular, R¹ to R¹⁰, G₁, G₂ and m are as defined above unless otherwise indicated. Furthermore, and unless explicitly otherwise stated, all reactions, reaction conditions, abbreviations and symbols have the meanings well known to a person of ordinary skill in organic chemistry.

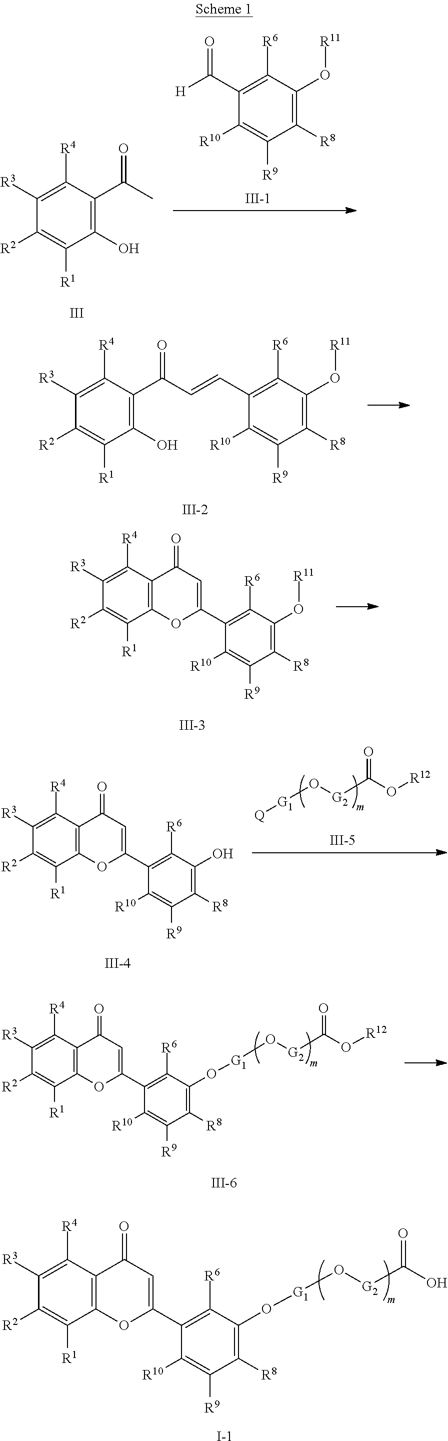

wherein R¹¹ is C₁₋₆alkyl, PMB or MOM; R¹² is C₁₋₆alkyl.

A compound of formula III-2 can be prepared by condensation of a compound of formula III and a compound formula III-1 in the presence of a base, such as KOH, in a suitable solvent, such as ethanol. Cyclization of a compound of formula III-2 in the presence of I₂ in DMSO gives a compound of formula III-3. Deprotection of a compound of formula III-3 in the presence of TFA or BBr₃ gives a compound of formula III-4. Alkylation of a compound of formula III-4 with a compound of formula III-5 affords a compound of formula III-6 in the presence of a base, such as K₂CO₃ or Cs₂CO₃, in a suitable solvent, such as DMF or acetone. A compound of formula I-1 is obtained by hydrolysis of a compound of formula III-6 in the presence of a base, such as LiOH, or an acid, such as TFA.

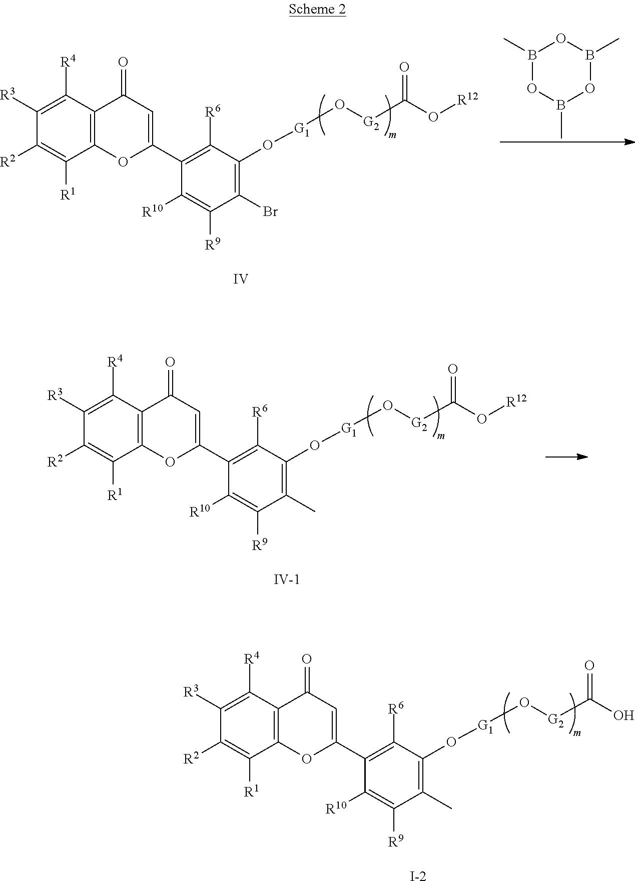

wherein R¹² is C₁₋₆alkyl.

A compound of formula IV-1 can be prepared by the reaction of a compound of formula IV with trimethylboroxine in the presence of a palladium catalyst. A compound of formula I-2 can be prepared by hydrolysis of a compound of formula IV-1 in the presence of a base, such as LiOH, or an acid, such as TFA.

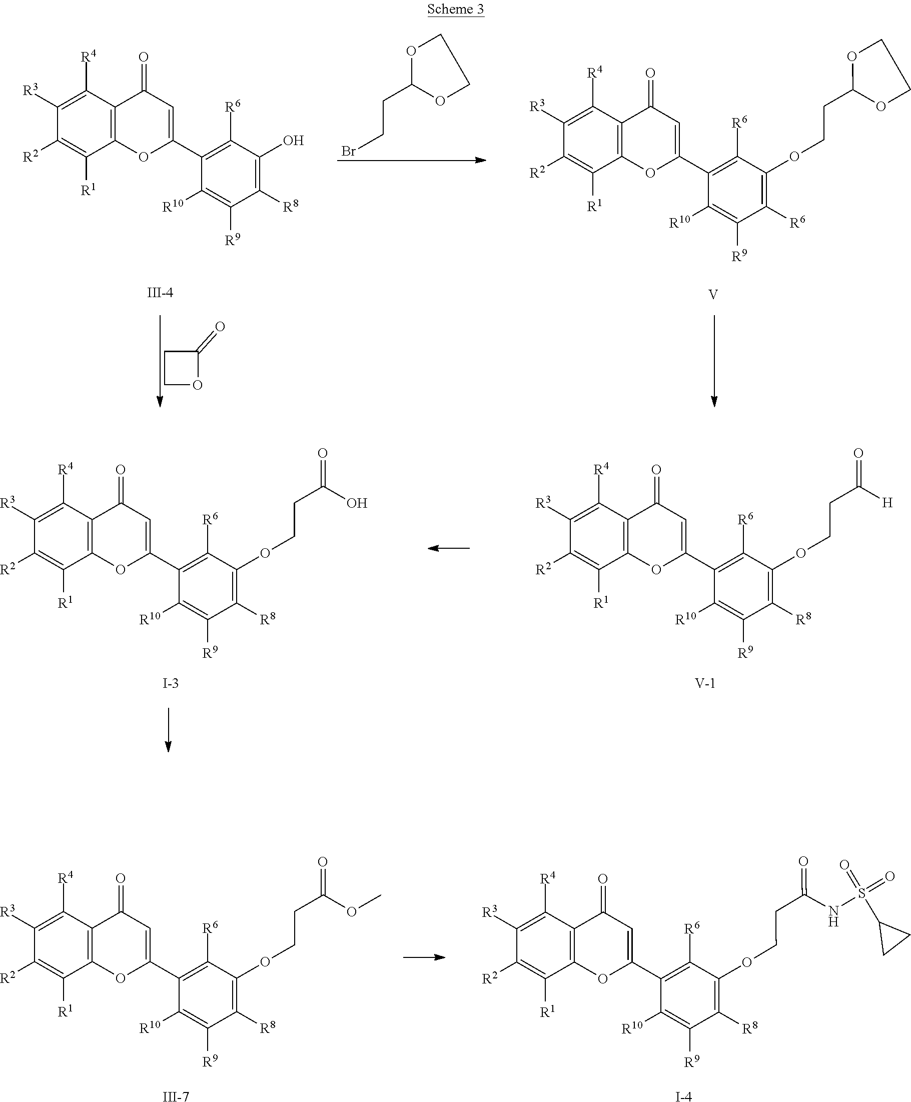

A compound of formula I-3 can be prepared directly by reaction of a compound of formula III-4 with oxetan-2-one in the presence of a base, such as sodium hydride, in a suitable solvent, such as DMF. Alternatively, alkylation of a compound of formula III-4 with 2-(2-bromoethyl)-1,3-dioxolane in the presence of a base, such as K₂CO₃, Cs₂CO₃ in the presence of a suitable solvent, such as DMF or acetone, gives a compound of formula V. Deprotection of compound of formula V in the presence of TFA affords a compound of formula V-1. Oxidation of a compound of formula V-1 using NaClO₂ gives compound of formula I-3. Treatment of a compound of formula I-3 with thionyl chloride in the presence of methanol affords a compound of formula III-7. Reaction of a compound of formula III-7 with sulfonamide in the presence of a Lewis acid, such as titanium tetrachloride, gives a compound of formula I-4.

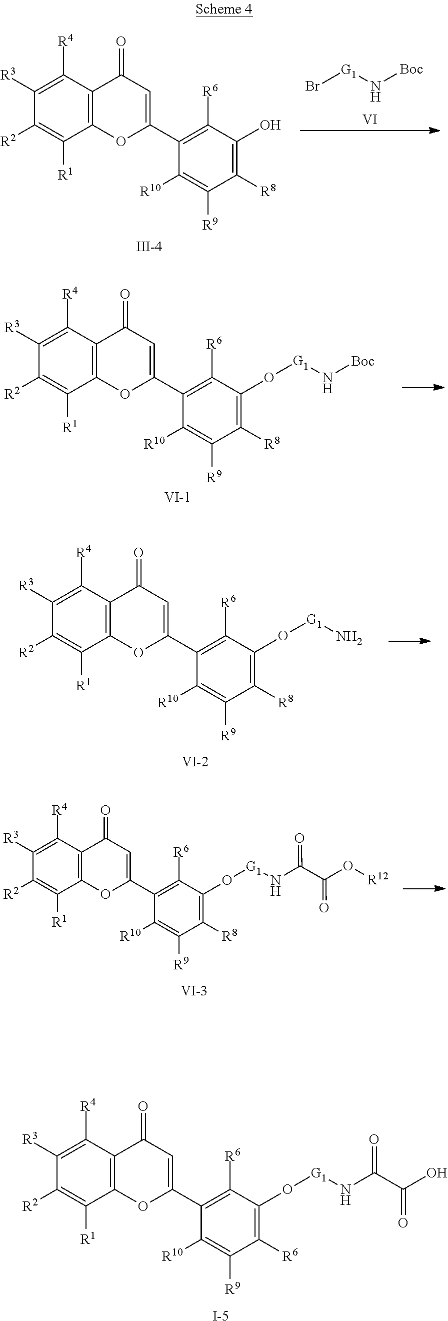

wherein R¹² is C₁₋₆alkyl.

Alkylation of a compound of formula III-4 with bromide VI in the presence of a base, such as t-BuOK, in a suitable solvent, such as DMF, gives a compound of formula VI-1. Deprotection of a compound of formula VI-1 in the presence of an acid, such as TFA, gives a compound of formula VI-2. Reaction of a compound of formula VI-2 with ethyl 2-chloro-2-oxo-acetate in a suitable organic base, such as TEA, affords a compound of formula VI-3. Hydrolysis of a compound of formula VI-3 in the presence of a base, such as LiOH or NaOH, affords a compound for formula I-5.

This invention also relates to a process for the preparation of a compound of formula (I) comprising at least one of the following steps:

(a) Hydrolysis of a compound of formula (III-6),

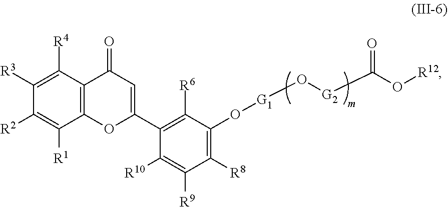

in the presence of a base or an acid;
(b) Hydrolysis of a compound of formula (IV-1),

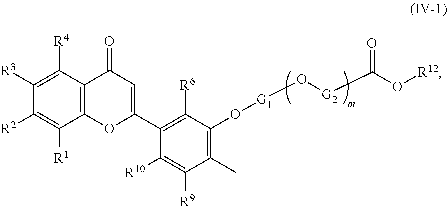

in the presence of a base or an acid;
(c) Reaction of a compound of formula (III-4),

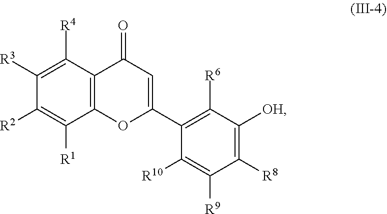

with oxetan-2-one in the presence of a base;
(d) Oxidation of a compound of formula (VI-1),

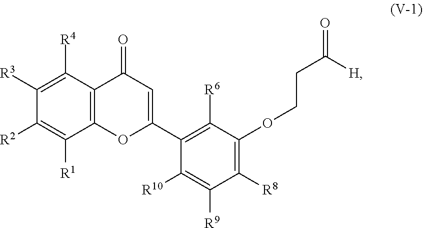

with NaClO₂;
(e) Reaction of a compound of formula (III-7),

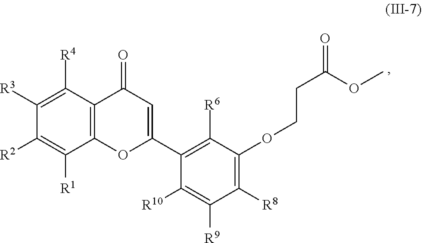

with sulfonamide in the presence of a Lewis acid;
(f) Hydrolysis of a compound of formula (VI-3),

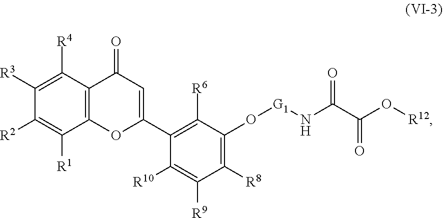

in the presence of a base; wherein R¹ to R⁴, R⁶, R⁸ to R¹⁰, G₁, G₂ and m are defined as any one of claims 1 to 3; R¹² is C₁₋₆alkyl.
The base in step (a) can be for example LiOH;
The acid in step (a) can be for example TFA;
The base in step (b) can be for example LiOH;
The acid in step (b) can be for example TFA;
The base in step (c) can be for example sodium hydride;
The Lewis acid in step (e) can be for example titanium tetrachloride;
The base in step (f) can be for example LiOH.

A compound of formula (I) or (II) when manufactured according to the above process is also an object of the invention.

The compound of this invention also shows good safety and PK profile.

### Pharmaceutical Compositions and Administration

The invention also relates to a compound of formula (I) or (II) for use as therapeutically active substance. Another embodiment provides pharmaceutical compositions or medicaments containing the compounds of the invention and a therapeutically inert carrier, diluent or excipient, as well as methods of using the compounds of the invention to prepare such compositions and medicaments. In one example, compounds of formula (I) or (II) may be formulated by mixing at ambient temperature at the appropriate pH, and at the desired degree of purity, with physiologically acceptable carriers, i.e., carriers that are non-toxic to recipients at the dosages and concentrations employed into a galenical administration form. The pH of the formulation depends mainly on the particular use and the concentration of compound, but preferably ranges anywhere from about 3 to about 8. In one example, a compound of formula (I) or (II) is formulated in an acetate buffer, at pH 5. In another embodiment, the compounds of formula (I) or (II) are sterile. The compound may be stored, for example, as a solid or amorphous composition, as a lyophilized formulation or as an aqueous solution.

Compositions are formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “effective amount” of the compound to be administered will be governed by such considerations, and is the minimum amount necessary to inhibit cccDNA in HBV patients, consequently lead to the reduction of HBsAg and HBeAg (HBV e antigen) in serum. For example, such amount may be below the amount that is toxic to normal cells, or the mammal as a whole.

In one example, the pharmaceutically effective amount of the compound of the invention administered parenterally per dose will be in the range of about 0.1 to 100 mg/kg, alternatively about 0.1 to 50 mg/kg of patient body weight per day, with the typical initial range of compound used being 0.3 to 15 mg/kg/day. In another embodiment, oral unit dosage forms, such as tablets and capsules, preferably contain from about 25 to about 1000 mg of the compound of the invention.

The compounds of the invention may be administered by any suitable means, including oral, topical (including buccal and sublingual), rectal, vaginal, transdermal, parenteral, subcutaneous, intraperitoneal, intrapulmonary, intradermal, intrathecal and epidural and intranasal, and, if desired for local treatment, intralesional administration. Parenteral infusions include intramuscular, intravenous, intraarterial, intraperitoneal, or subcutaneous administration.

The compounds of the present invention may be administered in any convenient administrative form, e.g., tablets, powders, capsules, solutions, dispersions, suspensions, syrups, sprays, suppositories, gels, emulsions, patches, etc. Such compositions may contain components conventional in pharmaceutical preparations, e.g., diluents, carriers, pH modifiers, sweeteners, bulking agents, and further active agents.

A typical formulation is prepared by mixing a compound of the present invention and a carrier or excipient. Suitable carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound of the present invention or pharmaceutical composition thereof) or aid in the manufacturing of the pharmaceutical product (i.e., medicament).

An example of a suitable oral dosage form is a tablet containing about 25 to 500 mg of the compound of the invention compounded with about 90 to 30 mg anhydrous lactose, about 5 to 40 mg sodium croscarmellose, about 5 to 30 mg polyvinylpyrrolidone (PVP) K30, and about 1 to 10 mg magnesium stearate. The powdered ingredients are first mixed together and then mixed with a solution of the PVP. The resulting composition can be dried, granulated, mixed with the magnesium stearate and compressed to tablet form using conventional equipment. An example of an aerosol formulation can be prepared by dissolving the compound, for example 5 to 400 mg, of the invention in a suitable buffer solution, e.g. a phosphate buffer, adding a tonicifier, e.g. a salt such sodium chloride, if desired. The solution may be filtered, e.g., using a 0.2 micron filter, to remove impurities and contaminants.

An embodiment, therefore, includes a pharmaceutical composition comprising a compound of Formula (I) or (II), or pharmaceutically acceptable salt or enantiomer or diastereomer thereof.

In a further embodiment includes a pharmaceutical composition comprising a compound of formula (I) or (II), or pharmaceutically acceptable salt or enantiomer or diastereomer thereof, together with a pharmaceutically acceptable carrier or excipient.

Another embodiment includes a pharmaceutical composition comprising a compound of formula (I) or (II), or pharmaceutically acceptable salt or enantiomer or diastereomer thereof for use in the treatment of HBV infection.

### Indications and Methods of Treatment

The compounds of the invention can inhibit cccDNA and have anti-HBV activity. Accordingly, the compounds of the invention are useful for the treatment or prophylaxis of HBV infection.

The invention relates to the use of a compound of formula (I) or (II) for the inhibition of cccDNA.

The invention also relates to the use of a compound of formula (I) or (II) for the inhibition of HBeAg.

The invention further relates to the use of a compound of formula (I) or (II) for the inhibition of HBsAg.

The invention relates to the use of a compound of formula (I) or (II) for the inhibition of HBV DNA.

The invention relates to the use of a compound of formula (I) or (II) for use in the treatment or prophylaxis of HBV infection.

The use of a compound of formula (I) or (II) for the preparation of medicaments useful in the treatment or prophylaxis diseases that are related to HBV infection is an object of the invention.

The invention relates in particular to the use of a compound of formula (I) or (II) for the preparation of a medicament for the treatment or prophylaxis of HBV infection.

Another embodiment includes a method for the treatment or prophylaxis of HBV infection, which method comprises administering an effective amount of a compound of Formula (I) or (II), or enantiomers, diastereomers, prodrugs or pharmaceutically acceptable salts thereof.

### EXAMPLES

The invention will be more fully understood by reference to the following examples. They should not, however, be construed as limiting the scope of the invention.

Abbreviations used herein are as follows:
ACN: acetonitrile
BBr3: boron tribromide
DMAP: 4-dimethylaminopyridine
DMF: N,N-dimethylformamide
IC50: the molar concentration of an inhibitor, which produces 50% of the maximum possible response for that inhibitor.
FBS: fetal bovine serum
HPLC: high performance liquid chromatography
MS (ESI): mass spectroscopy (electron spray ionization)
Ms: methylsulfonyl
obsd.: observed
PE: petroleum ether
EtOAc: ethyl acetate
AcOH: acetic acid
THF: tetrahydrofuran
TFA: trifluoroacetic acid
TEA: triethyl amine
DIPEA: N,N-Diisopropylethylamine
DIAD: Diisopropyl azodicarboxylate
Ts: p-tolylsulfonyl
δ: chemical shift
V/V: volume ratio
BOC: butyloxylcarbonyl
PMB: p-methoxybenzyl
MOM: methoxymethyl
TMS: trimethylsilyl
hr(s): hour(s)
min(s): minute(s)
TBS: tert-butyldimethylsilane

### General Experimental Conditions

Intermediates and final compounds were purified by flash chromatography using one of the following instruments: i) Biotage SP1 system and the Quad 12/25 Cartridge module. ii) column chromatography on silica gel combi-flash chromatography instrument. Silica gel Brand and pore size: i) KP-SIL 60 Å, particle size: 40-60 μm; ii) CAS registry NO: Silica Gel: 63231-67-4, particle size: 47-60 micron silica gel; iii) ZCX from Qingdao Haiyang Chemical Co., Ltd, pore: 200-300 or 300-400.

Intermediates and final compounds were purified by preparative HPLC on reversed phase column using X Bridge™ Perp C₁₈ (5 μm, OBD™ 30×100 mm) column or SunFire™ Perp C₁₈ (5 μm, OBD™ 30×100 mm) column.

LC/MS spectra were obtained using a Waters UPLC-SQD Mass. Standard LC/MS conditions were as follows (running time 3 minutes):
Acidic condition: A: 0.1% formic acid and 1% acetonitrile in H₂O; B: 0.1% formic acid in acetonitrile;
Basic condition: A: 0.05% NH₃—H₂O in H₂O; B: acetonitrile.

Mass spectra (MS): generally only ions which indicate the parent mass are reported, and unless otherwise stated the mass ion quoted is the positive mass ion (M+H)⁺.

NMR spectra were obtained using Bruker Avance 400 MHz.

All reactions involving air-sensitive reagents were performed under an argon atmosphere.

Reagents were used as received from commercial suppliers without further purification unless otherwise noted.

### PREPARATIVE EXAMPLES

### Intermediate AA

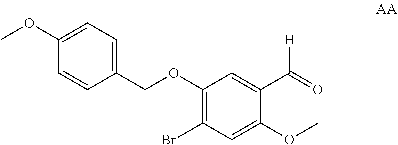

### Step 1. Synthesis of 4-bromo-5-hydroxy-2-methoxy-benzaldehyde

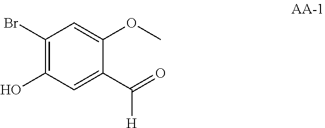

To a solution of 5-hydroxy-2-methoxy-benzaldehyde (1.6 g) in dry THF (20 mL) and tetrachloroethylene (40 mL) at 0° C. was added N-bromosuccinimide (1.87 g, 10.5 mmol) in small portions. After being stirred at 50° C. for 16 hrs, the reaction mixture was poured into water (150 mL). The resulting solution was extracted with DCM (100 mL) three times. The combined organics layer was dried over anhydrous Na₂SO₄, filtered and concentrated in vacuo. The resulting oil was purified by flash column chromatography (eluting with EtOAc/PE=1/5) to give 4-bromo-5-hydroxy-2-methoxy-benzaldehyde (1.0 g, compound AA-1) as a brown solid. MS obsd. (ESI⁺): 231.0 [(M+H)⁺], 233.0 [(M+2+H)⁺].

### Step 2. Synthesis 4-bromo-2-methoxy-5-[(4-methoxyphenyl)methoxy]benzaldehyde

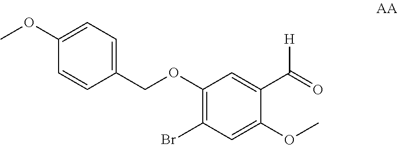 [SMILES: COc1ccc(COc2cc(C=O)c(OC)cc2Br)cc1]

To a solution of 4-bromo-5-hydroxy-2-methoxy-benzaldehyde (1.0 g, AA-1) in dry DMF (20 mL) at 0° C. was added sodium hydride (180 mg) in small portions. The reaction mixture was stirred at 0° C. for 0.5 hr. Then 4-methoxybenzylchloride (0.61 mL) was added dropwise. After being stirred at 25° C. for 16 hrs, the reaction mixture was poured into ice/water (150 g), extracted with EtOAc/THF(V/V)=4/1 (100 mL) three times. The combined organic layer was washed with brine (50 mL), dried over anhydrous sodium sulfate, filtered and concentrated in vacuo. The crude product was purified by flash silica gel chromatography (eluting with EtOAc/PE=1/10) to give 4-bromo-2-methoxy-5-[(4-methoxyphenyl)methoxy]benzaldehyde (800 mg, Intermediate AA) as a white solid. MS obsd. (ESI⁺): 373.0 [(M+Na)⁺], 375.0 [(M+2+Na)⁺].

### Intermediate AB

### Step 1. Synthesis of 4-chloro-5-hydroxy-2-methoxy-benzaldehyde

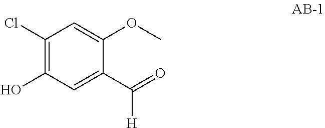

To a solution of 5-hydroxy-2-methoxy-benzaldehyde (0.5 g, 3.29 mmol) in dry THF (20 mL) and tetrachloroethylene (20 mL) was added N-chlorosuccinimide (0.53 g). After being stirred at 25° C. for 16 hrs, the reaction mixture was concentrated in vacuo. The resulting reaction mixture was diluted with EtOAc (100 mL), washed with brine (50 mL), dried over anhydrous sodium sulfate, filtered and concentrated in vacuo. The residue was purified by flash silica gel chromatography (eluting with EtOAc/PE=1/4) to give 4-chloro-5-hydroxy-2-methoxy-benzaldehyde (450 mg, compound AB-1) as a yellow solid. MS obsd. (ESI⁺): 187.1 [(M+H)⁺], 189.1 [(M+2+H)⁺].

### Step 2. Synthesis of 4-chloro-2-methoxy-5-(methoxymethoxy)benzaldehyde

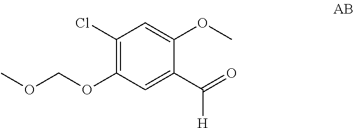

To a solution of 4-chloro-5-hydroxy-2-methoxy-benzaldehyde (0.4 g, AB-1) in dry DMF (10 mL) at 0° C. was added sodium hydride (154 mg, 3.86 mmol) in small portions. The reaction mixture was stirred at 0° C. for 0.5 h, bromomethyl methyl ether (482 mg) was added dropwise. After being stirred at 25° C. for 6 hrs, the reaction mixture was poured into ice/water (50 g), extracted with EtOAc (30 mL) three times. The combined organic layer was washed with brine (25 mL), dried over anhydrous sodium sulfate, filtered and concentrated in vacuo. The crude product was purified by flash silica gel chromatography (eluting with EtOAc/PE=1/10) to give 4-chloro-2-methoxy-5-(methoxymethoxy)benzaldehyde (280 mg, Intermediate AB) as a white solid. MS obsd. (ESI⁺): 231.1 [(M+H)⁺], 233.1 [(M+2+H)⁺].

### Intermediate AC

### 5-Hydroxy-2,4-dimethoxy-benzaldehyde

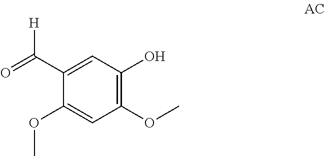

To a solution of sodium methoxide (4.21 g, 77.9 mmol) in DMF (50 ml) was added copper (I) iodide (247 mg, 1.3 mmol) and 2-bromo-5-hydroxy-4-methoxybenzaldehyde (3 g, 13 mmol). The resulting mixture was refluxed for 4 hrs. After it was cooled to room temperature and filtered, the filtrate was transferred to an Erlenmeyer flask containing 30 g of ice and 10 mL of concentrated HCl and extracted with EtOAc (50 mL) three times. The organic layer was dried over Na₂SO₄ and concentrated in vacuo to give of 5-hydroxy-2,4-dimethoxy-benzaldehyde (1.8 g, Intermediate AC) as a yellow solid. MS obsd. (ESI⁺) [(M+H)⁺]:183.

### Intermediate AD

### 3-[Tert-butyl(dimethyl)silyl]oxy-2-methoxy-benzaldehyde

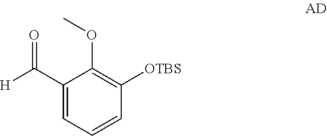 [SMILES: COc1c(C=O)cccc1O[Si](C)(C)C(C)(C)C]

### Step 1. Synthesis of 3-[tert-butyl(dimethyl)silyl]oxy-2-hydroxy-benzaldehyde

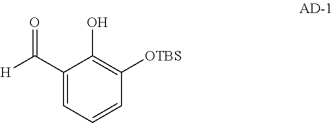 [SMILES: CC(C)(C)[Si](C)(C)Oc1cccc(C=O)c1O]

To a solution of tert-butyldimethylchlorosilane (7.19 mL) and imidazole (3.75 g) in DCM (100 mL) was added 2,3-dihydroxybenzaldehyde (5.07 g). After being stirred at 25° C. for 16 hrs, the reaction mixture was diluted with DCM (100 mL). The organic phase was washed with water (150 mL), brine (150 mL), dried over anhydrous sodium sulfate, filtered and concentrated in vacuo. The residue was purified by flash silica gel chromatography (eluting with EtOAc/PE=1/20) to give 3-[tert-butyl(dimethyl)silyl]oxy-2-hydroxy-benzaldehyde (7.2 g, AD-1) as a yellow oil. MS obsd. (ESI⁺): 253.1 [(M+H)⁺], 275.1 [(M+Na)⁺].

### Step 2. Synthesis of 3-[tert-butyl(dimethyl)silyl]oxy-2-methoxy-benzaldehyde

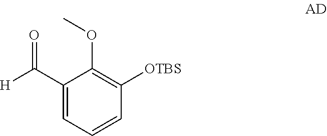 [SMILES: COc1c(C=O)cccc1O[Si](C)(C)C(C)(C)C]

To a solution of 3-[tert-butyl(dimethyl)silyl]oxy-2-hydroxy-benzaldehyde (7.2 g) and potassium carbonate (6.3 g) in DMF (70 mL) was added iodomethane (6.56 g). After being stirred at 25° C. for 16 hrs, the reaction mixture was filtered through celite. The filtered cake was washed with EtOAc (100 mL). The combined filtrate was concentrated in vacuo. The residue was diluted with EtOAc (200 mL), washed with brine (50 mL), dried over anhydrous sodium sulfate, filtered and concentrated in vacuo. The crude product was purified by flash silica gel chromatography (eluting with EtOAc/PE=1/10) to give 3-[tert-butyl(dimethyl)silyl]oxy-2-methoxy-benzaldehyde (6 g, Intermediate AD) as a colorless oil. MS obsd. (ESI⁺): 267.1 [(M+H)⁺], 289.1 [(M+Na)⁺].

### Intermediate AE

### 5-Hydroxy-2-methoxy-benzaldehyde

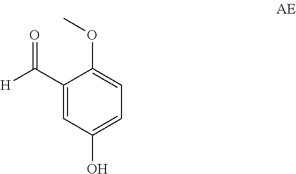

### Step 1. Synthesis of 2,5-dimethoxybenzaldehyde

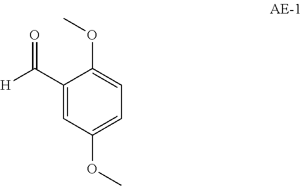

To a solution of 2,5-dihydroxybenzaldehyde (2.76 g) and potassium carbonate (11.06 g) in dry DMF (40 mL) was added iodomethane (4.98 mL). After being stirred at 25° C. for 16 hrs, the reaction mixture was filtered through celite. The filtered cake was washed with DCM (100 mL), the combined filtrate was concentrated in vacuo. The residue was diluted with EtOAc (200 mL), washed with brine (50 mL), dried over anhydrous sodium sulfate, filtered and concentrated in vacuo. The crude product was purified by flash silica gel chromatography (eluting with EtOAc/PE=1/10) to give 2,5-dimethoxybenzaldehyde (2.4 g. compound AE-1) as a white solid. MS obsd. (ESI⁺): 167.1 [(M+H)⁺], 189.1 [(M+Na)⁺].

### Step 2. Synthesis of 5-hydroxy-2-methoxy-benzaldehyde

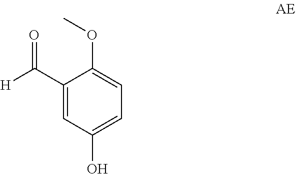

To 2,5-dimethoxybenzaldehyde (1.2 g, compound AE-1) at 0° C. was added sulfuric acid (6.5 mL). After being stirred at 60° C. for 72 hrs, the reaction mixture was cooled to room temperature and poured into ice (250 g). The precipitated oil was extracted with diethyl ether (50 mL) three times. The diethyl ether solution was washed with 20 mL of 5% sodium hydroxide, the water solution was acidified to pH=5 with 2 M HCl at 0° C., extracted with diethyl ether (50 mL) three times. The combined organics were washed with brine (50 mL), dried over anhydrous Na₂SO₄, filtered and concentrated in vacuo. The crude product was purified by flash column chromatography (eluting with EtOAc/PE=2/3) to give 5-hydroxy-2-methoxy-benzaldehyde (300 mg, Intermediate AE) as a yellow solid. MS obsd. (ESI⁺): 153.1 [(M+H)⁺], 175.1 [(M+Na)⁺].

### Intermediate AF

### 4-Bromo-3-hydroxy-2-methoxy-benzaldehyde

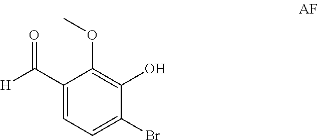

### Step 1. Synthesis of 3-hydroxy-2-methoxy-benzaldehyde

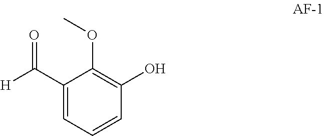

To a solution of 3-[tert-butyl(dimethyl)silyl]oxy-2-methoxy-benzaldehyde (1.1 g) in methanol (10 mL) was added a solution of hydrochloric acid (10.0 mL) in methanol (4 M). After being stirred at 25° C. for 16 hrs, the reaction mixture was concentrated in vacuo. The crude product was diluted with EtOAc (100 mL), washed with brine (100 mL), dried over anhydrous Na₂SO₄, filtered and concentrated in vacuo to give 3-hydroxy-2-methoxy-benzaldehyde (550 mg, compound AF-1) as a brown solid which was used for next step without purification. MS obsd. (ESI⁺): 153.1 [(M+H)⁺], 175.1 [(M+Na)⁺].

### Step 2. Synthesis of 4-bromo-3-hydroxy-2-methoxy-benzaldehyde

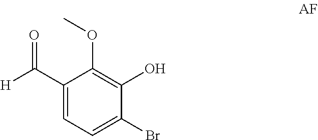

To a solution of 3-hydroxy-2-methoxy-benzaldehyde (0.32 g, compound AF-1) in dry DCM (25 mL) was added N-bromosuccinimide (0.45 g). After being stirred at 25° C. for 16 hrs, the reaction mixture was diluted with DCM (25 mL), washed with brine (25 mL), dried over anhydrous sodium sulfate, filtered and concentrated in vacuo. The crude product was purified by flash silica gel chromatography (eluting with EtOAc/PE=1/4) to give 4-bromo-3-hydroxy-2-methoxy-benzaldehyde (420 mg, Intermediate AF) as a white solid. MS obsd. (ESI⁺): 231.0 [(M+H)⁺], 233.0 [(M+2+H)⁺].

### Intermediate AG

### 2-Methoxy-3-(methoxymethoxy)-4-methyl-benzaldehyde

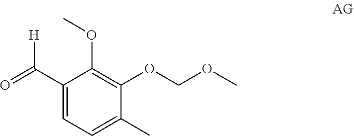

### Step 1. Synthesis of 3-hydroxy-2-methoxy-4-methyl-benzaldehyde

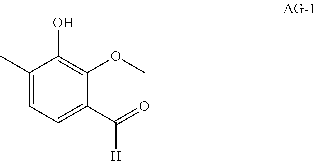

To a solution of 4-bromo-3-hydroxy-2-methoxy-benzaldehyde (420.0 mg) in 1,4-dioxane (20 mL) were added [1,1′-bis(diphenylphosphino)ferrocene]dichloropalladium(II) (110.4 mg), cesium carbonate (983 mg). The mixture was degassed with N₂ for 3 times. Then trimethylboroxine (378.81 mg) was added. After being stirred at 100° C. for 16 hrs, the reaction mixture was diluted with EtOAc (30 mL), filtered through celite, acidified to pH=3 with 4 M HCl, extracted with EtOAc (30 mL) three times. The combined organics were dried over anhydrous Na₂SO₄, filtered and concentrated in vacuo. The crude product was purified by flash silica gel chromatography (eluting with EtOAc/PE=1/5) to give 3-hydroxy-2-methoxy-4-methyl-benzaldehyde (200 mg, compound AG-1) as a yellow solid. MS obsd. (ESI⁺): 167.1 [(M+H)⁺].

### Step 2. Synthesis of 2-methoxy-3-(methoxymethoxy)-4-methyl-benzaldehyde

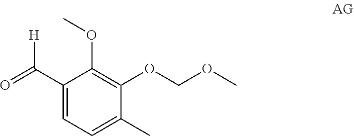

To a solution of 3-hydroxy-2-methoxy-4-methyl-benzaldehyde (180.0 mg) in dry DMF (10 mL) was added sodium hydride (58 mg) in small portions at 0° C. After the mixture was stirred for 0.5 h, then bromomethyl methyl ether (180.71 mg) was added dropwise. After being stirred at 25° C. for 16 hrs, the reaction mixture was poured into ice/water (50 g), extracted with EtOAc (30 mL) three times. The combined organic layer was washed with brine (25 mL), dried over anhydrous sodium sulfate, filtered and concentrated in vacuo. The crude product was purified by flash silica gel chromatography (eluting with EtOAc/PE=1/10) to give 2-methoxy-3-(methoxymethoxy)-4-methyl-benzaldehyde (200 mg, Intermediate AG) as a white solid. MS obsd. (ESI⁺): 211.1 [(M+H)⁺].

### Intermediate AH

### Step 1. Synthesis of 4-chloro-3-hydroxy-2-methoxy-benzaldehyde

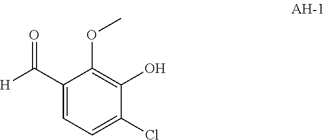

To a solution of 3-hydroxy-2-methoxy-benzaldehyde (2.0 g) in dry THF (37.5 mL) and tetrachloroethylene (75 mL) was added N-chlorosuccinimide (1.93 g) in small portions at 0° C. After addition, the mixture was degassed with N₂ for 3 times. After being stirred at 50° C. for 48 hrs, the reaction mixture was poured into water (150 mL), and the resulting biphasic mixture was extracted with DCM (100 mL) three times. The combined organics were dried over anhydrous Na₂SO₄, filtered and concentrated in vacuo. The resulting oil was purified by flash silica gel column chromatography (eluting with EtOAc/PE=1/5) to give 4-chloro-3-hydroxy-2-methoxy-benzaldehyde (1.7 g, compound AH-1) as a white solid. MS obsd. (ESI⁺): 187.1 [(M+H)⁺], 189.1 [(M+2+H)⁺].

### Step 2. Synthesis of 4-chloro-2-methoxy-3-[(4-methoxyphenyl)methoxy]benzaldehyde

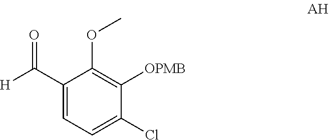

To a solution of 4-chloro-3-hydroxy-2-methoxy-benzaldehyde (1.7 g, compound AH-1) in dry DMF (30 mL) was added sodium hydride (398 mg) in small portions at 0° C. After being stirred at 0° C. for 0.5 h, a solution of 4-methoxybenzylchloride (1.35 mL) in dry DMF (10 mL) was added dropwise. After being stirred at 25° C. for 6 hrs, the reaction mixture was poured into ice/water (150 g) and extracted with EtOAc/THF=4/1 (100 mL) three times. The combined organic layer was washed with brine (150 mL), dried over anhydrous sodium sulfate, filtered and concentrated in vacuo. The crude product was purified by flash silica gel chromatography (eluting with EtOAc/PE=1/10) to give 4-chloro-2-methoxy-3-[(4-methoxyphenyl)methoxy]benzaldehyde (1.1 g, Intermediate AH) as a white solid. MS obsd. (ESI⁺): 329.0 [(M+Na)⁺], 331.1 [(M+2+Na)⁺].

### Intermediate AI

### 2,4-Dimethoxy-3-[(4-methoxyphenyl)methoxy]benzaldehyde

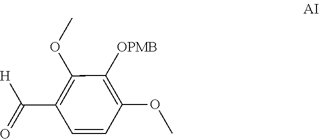

### Step 1. Synthesis of 3-hydroxy-2,4-dimethoxy-benzaldehyde

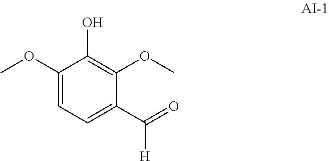

To a solution of 3-[tert-butyl(dimethyl)silyl]oxy-2,4-dimethoxy-benzaldehyde (3.8 g) in DCM (39 mL) at 0° C., was added a solution of hydrochloric acid (32 mL) in MeOH (4 M). After addition, the mixture was stirred 25° C. for 16 hrs. Then the reaction mixture was concentrated in vacuo, diluted with ethyl acetate (200 mL), washed with brine (50 mL) two times, dried over anhydrous sodium sulfate, filtered and concentrated in vacuo to give 3-hydroxy-2,4-dimethoxy-benzaldehyde (2.2 g, compound AI-1) as a yellow oil. MS obsd. (ESI⁺): 183.1 [(M+H)⁺].

### Step 2. Synthesis of 2,4-dimethoxy-3-[(4-methoxyphenyl)methoxy]benzaldehyde

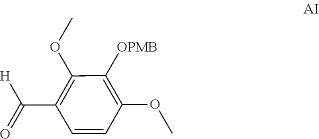

To a solution of 3-hydroxy-2,4-dimethoxy-benzaldehyde (2.2 g, compound AI-1) in DMF (50 mL) at 25° C. were added potassium iodide (0.13 mL), dipotassium carbonate (2.5 g) and 4-methoxybenzylchloride (2.46 mL). After being stirred at 60° C. for 16 hrs, the reaction mixture was poured into ice/water (250 mL), extracted with EtOAc (100 mL) three times. The combined organic layer was washed with brine (100 mL) four times. The organic phase was dried over anhydrous sodium sulfate, filtered and concentrated in vacuo. The crude product was purified by flash silica gel chromatography (eluting with EtOAc/PE=3/7) to give 2,4-dimethoxy-3-[(4-methoxyphenyl)methoxy]benzaldehyde (3 g, Intermediate AI) as a yellow solid. MS obsd. (ESI⁺): 325.1 [(M+Na)⁺].

### Intermediate AJ

### Synthesis of 4-bromo-2-hydroxy-5-(methoxymethoxy)benzaldehyde

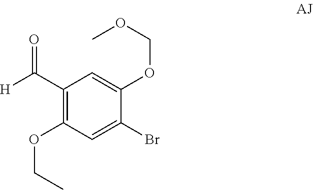 [SMILES: CCOc1cc(Br)c(OCOC)cc1C=O]

### Step 1. Synthesis of 4-bromo-2-hydroxy-5-(methoxymethoxy)benzaldehyde

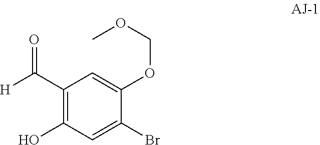

To a solution of 4-bromo-2,5-dihydroxy-benzaldehyde (1.1 g, 5.07 mmol) in THF (6.29 mL) was added N,N-diisopropylethylamine (1.77 mL, 10.14 mmol). The reaction was stirred at 0° C. for 10 mins. Then bromomethyl methyl ether (0.41 mL, 5.07 mmol) was added. After being stirred at 0° C. for 1 hr, the reaction mixture was diluted with water (20 mL) and extracted with EtOAc (30 mL) three times. The combined organic layer was washed with brine (30 mL) two times, then the organic layer was dried with MgSO₄, filtered and concentrated. The crude was purified by flash column chromatography (eluting with EtOAc/PE=1/10) to afford 4-bromo-2-hydroxy-5-(methoxymethoxy)benzaldehyde (200 mg, 15% yield, compound AJ-1) as a white solid. MS obsd. (ESI⁺): 261.0 [(M+H)⁺], 263.0 [(M+2+H)⁺].

### Step 2. Synthesis 4-bromo-2-ethoxy-5-(methoxymethoxy)benzaldehyde

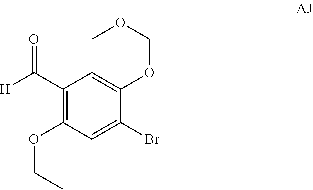 [SMILES: CCOc1cc(Br)c(OCOC)cc1C=O]

To a mixture of 4-bromo-2-hydroxy-5-(methoxymethoxy)benzaldehyde (100.0 mg), potassium carbonate (158 mg) in ACN (3 mL) was added iodoethane (0.06 mL). The mixture was stirred at 25° C. for 1.5 h. The reaction mixture was added water (20 mL) and extracted with EtOAc (30 mL) three times. The combined organic layer was washed with brine, dried over anhydrous sodium sulfate and concentrated. The residue was purified by flash column chromatography (eluting with EtOAc/PE=1/10) to give 4-bromo-2-ethoxy-5-(methoxymethoxy)benzaldehyde (90 mg, 81%, Intermediate AJ) as a yellow solid. ¹H NMR (400 MHz, DMSO-d₆): δ ppm 10.11-10.45 (m, 1H), 7.49-7.67 (m, 1H), 7.44 (s, 1H), 5.14-5.38 (m, 2H), 4.05-4.32 (m, 2H), 3.36-3.54 (m, 3H), 1.25-1.46 (m, 3H). MS obsd. (ESI⁺): 289.0 [(M+H)⁺], 291.0 [(M+2+H)⁺].

### Intermediate AK

### 4-Bromo-2-(difluoromethoxy)-5-(methoxymethoxy)benzaldehyde

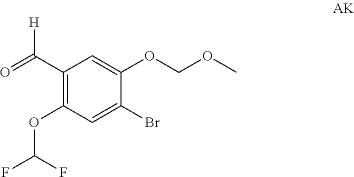 [SMILES: COCOc1cc(C=O)c(OC(F)F)cc1Br]

### Step 1. Synthesis of 4-bromo-2-hydroxy-5-(methoxymethoxy)benzaldehyde

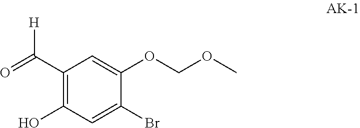

To a solution of 4-bromo-2,5-dihydroxy-benzaldehyde (3.45 g) in THF (40 mL) was added N,N-diisopropylethylamine (5.54 mL). The reaction was stirred at 25° C. for 10 min, then bromomethyl methyl ether (1.03 mL) was added. After being stirred at 25° C. for 1.5 h, the reaction mixture was diluted with water (20 mL). The solution was extracted with EtOAc (30 mL) three times. The combined organic layer was washed with brine (30 mL) two times, then the organic layer was dried with MgSO₄, filtered and concentrated. The crude was purified by flash column chromatography (eluting with EtOAc/PE=1/10) to afford 4-bromo-2-hydroxy-5-(methoxymethoxy)benzaldehyde (1.8 g, 43% yield, compound AK-1) as a white solid. MS obsd. (ESI⁺): 261.0 [(M+H)⁺], 263.0 [(M+2+H)⁺].

### Step 2. Synthesis of 5-bromo-2-(hydroxymethyl)-4-(methoxymethoxy)phenol

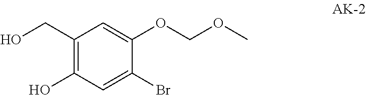

To the solution of 4-bromo-2-hydroxy-5-(methoxymethoxy)benzaldehyde (300.0 mg, compound AK-1) in THF (15 mL) at 0° C. was added sodium borohydride (65 mg). Then the mixture was stirred at 25° C. for 1 hr. The reaction mixture was poured into water (30 mL) and extracted with EtOAc (30 mL) three times. The combined organic layer was washed with brine, dried over anhydrous sodium sulfate and concentrated. The residue was purified by flash silica column chromatography (eluting with EtOAc/PE=1/10) to give 5-bromo-2-(hydroxymethyl)-4-(methoxymethoxy)phenol (200 mg, 66% yield, compound AD-2) as a yellow solid. MS obsd. (ESI⁺): 283.0 [(M+Na)⁺], 285.0 [(M+2+Na)⁺].

### Step 3. Synthesis of 4-bromo-2-(difluoromethoxy)-5-(methoxymethoxy)benzaldehyde

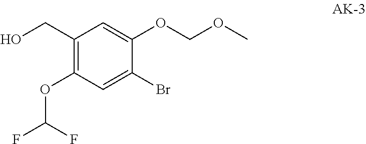 [SMILES: COCOc1cc(CO)c(OC(F)F)cc1Br]

To a solution of 5-bromo-2-(hydroxymethyl)-4-(methoxymethoxy)phenol (200.0 mg, compound AK-2) in DMF (3.3 mL) was added (2-chloro-2,2-difluoro-acetyl)oxysodium (127 mg) and cesium carbonate (743 mg). After being stirred at 80° C. for 3 hrs, the reaction mixture was added water (50 mL) and extracted with EtOAc (50 mL) three times. The combined organic layer was washed with brine, dried over anhydrous sodium sulfate and concentrated. The residue was purified by flash silica column chromatography (eluting with EtOAc/PE=1/4) to give 4-bromo-2-(difluoromethoxy)-5-(methoxymethoxy)benzaldehyde (160 mg, 68% yield, compound AK-3) as a yellow solid. MS obsd. (ESI⁺): 295.0 [(M−18+H)⁺], 297.0 [(M−18+2+H)⁺].

### Step 4. Synthesis of 4-bromo-2-(difluoromethoxy)-5-(methoxymethoxy)benzaldehyde (A4)

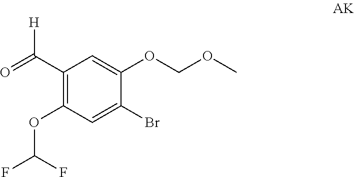 [SMILES: COCOc1cc(C=O)c(OC(F)F)cc1Br]

A mixture of [4-bromo-2-(difluoromethoxy)-5-(methoxymethoxy)phenyl]methanol (150.0 mg, compound AK-3) and manganese dioxide (416.53 mg) in DCM (2.5 mL) was stirred at 25° C. for 10 hrs. The mixture was filtered and concentrated. The residue was purified by flash column chromatography (eluting with EtOAc/PE=1/10) to give 4-bromo-2-(difluoromethoxy)-5-(methoxymethoxy)benzaldehyde (90 mg, 60% yield, Intermediate AK) as a yellow solid. ¹H NMR (400 MHz, DMSO-d₆): δ ppm 10.18 (s, 1H), 7.76 (s, 1H), 7.57 (s, 1H), 7.51 (s, 0.25H), 7.33 (s, 0.5H), 7.14 (s, 0.25H), 5.37 (s, 2H), 3.40 (d, J=5.5 Hz, 4H). MS obsd. (ESI⁺): 291.0 [(M−18+H)⁺], 293.0 [(M−18+2+H)⁺].

### Intermediate AL

### 4-Bromo-2-fluoro-5-(methoxymethoxy)benzaldehyde

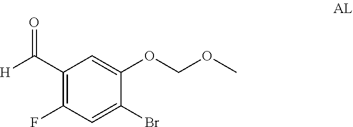

### Step 1. Synthesis of 4-bromo-2-fluoro-5-methoxy-benzaldehyde

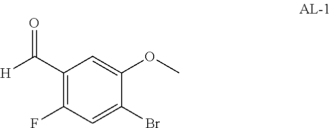

To a 25 mL round-bottom flask was added titanium tetrachloride (8.42 mL), followed by 2-bromo-4-fluoroanisole (3.15 g) in N₂. The stirred mixture was cooled in an ice water bath and treated dropwise with 1,1-dichlorodimethyl ether (6.95 mL). After being stirred for 1 h, water (20 mL) was added. The solution was extracted with EtOAc (50 mL) three times. The combined organic layer was washed with brine (50 mL) two times, dried with MgSO₄, filtered and concentrated. The crude was purified by flash column chromatography (eluting with EtOAc/PE=1/20) to afford 4-bromo-2-fluoro-5-methoxy-benzaldehyde (1.6 g, 44% yield, AL-1) as a white solid. MS obsd. (ESI⁺): 233.0 [(M+H)⁺], 234.9 [(M+H+2)⁺].

### Step 2. Synthesis of 4-bromo-2-fluoro-5-hydroxy-benzaldehyde

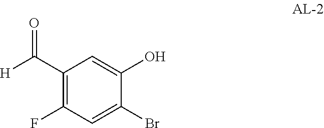 [SMILES: O=Cc1cc(O)c(Br)cc1F]

To a solution of 4-bromo-2-fluoro-5-methoxy-benzaldehyde (1.6 g, AL-1) in DCM (30 mL) at −78° C. was added boron tribromide (3.31 mL). After being stirred at −78° C. for 1 hr, water was added. The solution was extracted with DCM (50 mL) three times. The combined organic layer was washed with brine (50 mL) three times, then the organic layer was dried with MgSO₄, filtered and concentrated. The crude was purified by flash column chromatography (eluting with EtOAc/PE=1/4) to afford 4-bromo-2-fluoro-5-hydroxy-benzaldehyde (1.35 g, 89% yield, compound AL-2) as a white solid. MS obsd. (ESI⁺): 219.0 [(M+H)⁺], 220.9 [(M+H+2)⁺].

### Step 2. Synthesis of 4-bromo-2-fluoro-5-(methoxymethoxy)benzaldehyde

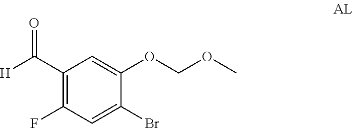

To a solution of 4-bromo-2-fluoro-5-hydroxy-benzaldehyde (300.0 mg, AL-2) in THF (6 mL) was added sodium hydride (82 mg), the reaction was stirred at 0° C. for 10 min. Then bromomethyl methyl ether (205.4 mg) was added. After being stirred at 0° C. for 1 h, the reaction mixture was diluted with water (20 mL) and extracted with EtOAc (30 mL) three times. The combined organic layer was washed with brine (30 mL) two times, then the organic layer was dried with MgSO₄, filtered and concentrated. The crude was purified by flash column chromatography (eluting with EtOAc/PE=1/10) to afford 4-bromo-2-fluoro-5-(methoxymethoxy)benzaldehyde (260 mg, 72% yield, Intermediate AE) as a white solid. MS obsd. (ESI⁺): 263.0 [(M+H)⁺], 265.0 [(M+H+2)⁺].

### Intermediate AM

### 4-Bromo-2-chloro-5-(methoxymethoxy)benzaldehyde

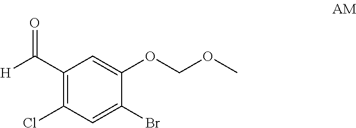

### Step 1. Synthesis of 4-bromo-2-chloro-5-hydroxy-benzaldehyde

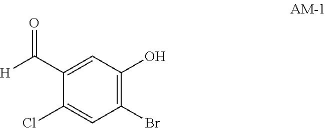 [SMILES: O=Cc1cc(O)c(Br)cc1Cl]

To a solution of 2-chloro-5-hydroxy-benzaldehyde (2.0 g) in chloroform (20 mL) was added bromine (2.0 g) and the mixture was stirred at 25° C. for 1 hrs. The reaction was concentrated to dryness and the residue was taken up in EtOAc (50 mL) and washed with water (50 mL) three times. The organic phase was dried over MgSO₄ and concentrated. The crude product was then purified by flash column chromatography (eluting with EtOAc/isohexane=1/4.

The desired fractions were concentrated in vacuo to give 4-bromo-2-chloro-5-hydroxy-benzaldehyde (1.50 g, compound AM-1) as a white solid MS obsd. (ESI⁺): 249.0 [(M+Me)⁺], 251.0 [(M+2+Me)⁺].

### Step 2. Synthesis of 4-bromo-2-chloro-5-(methoxymethoxy)benzaldehyde

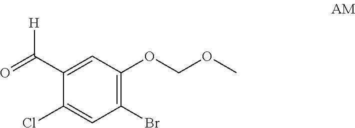

To a solution of 4-bromo-2-chloro-5-hydroxy-benzaldehyde (300.0 mg, AG-1) in THF (3 mL) was added sodium hydride (76 mg) and stirred for 20 min, followed by addition of bromomethyl methyl ether (238 mg). After being stirred for 0.5 h, the reaction mixture was quenched with water and concentrated in vacuo. The residue was purified by flash chromatography (eluting with EtOAc/PE=1/10) to give 4-bromo-2-chloro-5-(methoxymethoxy)benzaldehyde (325 mg, Intermediate AM) as colorless oil.

### Intermediate AN

### 3-[2-Bromo-5-formyl-4-(trifluoromethoxy)phenoxy]propanoic acid

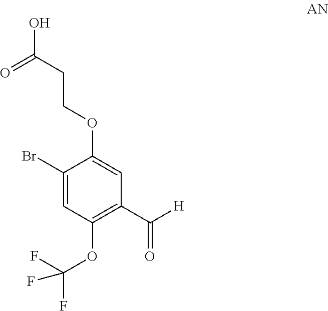 [SMILES: O=Cc1cc(OCCC(=O)O)c(Br)cc1OC(F)(F)F]

### Step 1. Synthesis of 2-bromo-4-methoxy-1-(trifluoromethoxy)benzene

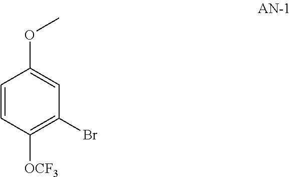

To a solution of 3-bromo-4-(trifluoromethoxy)phenol (4.0 g) in acetone (20 mL) were added potassium carbonate (8.6 g) and iodomethane (6.6 g) at r.t and then heated at 80° C. for 4 hrs. The mixture was quenched with water and extracted with EtOAc (50 mL) three times. The combined organic layer was concentrated and then purified by flash chromatography (eluting with EtOAc/PE=1/10) to give 2-bromo-4-methoxy-1-(trifluoromethoxy)benzene (3.5 g, compound AN-1) as a colorless oil.

### Step 2. Synthesis of 5-methoxy-2-(trifluoromethoxy)benzaldehyde

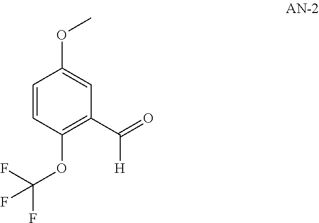

To a solution of 2-bromo-4-methoxy-1-(trifluoromethoxy)benzene (2.0 g, compound AN-1) in THF (33 mL) was added dropwise butyllithium solution (0.66 g) at −60° C. The mixture was added dimethylformamide (1.71 mL). After being warmed to r.t for 1 h, the reaction mixture was quenched by NH₄Cl (2 mL), diluted with water and extracted with EtOAc (80 mL) three times. The combined organic layer was washed by brine and dried over Na₂SO₄, concentrated and purified by flash chromatography (eluting with EtOAc/PE=1/10) to give 5-methoxy-2-(trifluoromethoxy)benzaldehyde (1.4 g, compound AN-2) as a colorless oil.

### Step 3. Synthesis of 4-bromo-5-methoxy-2-(trifluoromethoxy)benzaldehyde

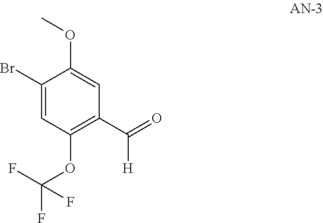 [SMILES: COc1cc(C=O)c(OC(F)(F)F)cc1Br]

To a solution of 5-methoxy-2-(trifluoromethoxy)benzaldehyde (3.8 g, compound AN-2) in ACN (100 mL) was added N-bromosuccinimide (3.1 g) and benzoyl peroxide (0.21 g) at r.t. After being heated at 90° C. for 2 hrs, the reaction mixture was cooled with ice-water, poured into water and extracted with EtOAc (120 mL) three times. The organic phase was washed with water, dried over Na₂SO₄, concentrated. The residue was purified by flash chromatography (eluting with EtOAc/PE=1/10) to give 4-bromo-5-methoxy-2-(trifluoromethoxy)benzaldehyde (2.6 g, compound AN-3) as a light yellow solid. MS obsd. (ESI⁺): 299.0 [(M+H)⁺], 301.0 [(M+2+H)⁺].

### Step 4. Synthesis of 4-bromo-5-hydroxy-2-(trifluoromethoxy)benzaldehyde

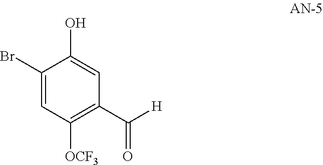 [SMILES: O=Cc1cc(O)c(Br)cc1OC(F)(F)F]

To a solution of 4-bromo-5-methoxy-2-(trifluoromethoxy)benzaldehyde (400.0 mg, compound AN-4) in DCM (8 mL) were added boron tribromide (335.1 mg) at −65° C. for 0.5 h and warmed up to r.t for 3 hrs. The mixture dropwise added to ice water and extracted with DCM (20 mL) three times. The combined organic layer was dried over Na₂SO₄, concentrated in vacuo. The residue was purified by flash chromatography (eluting with EtOAc/PE=1/10) to give 4-bromo-5-hydroxy-2-(trifluoromethoxy)benzaldehyde (300 mg, AN-5) as a brown oil. MS obsd. (ESI⁺): 299.0 [(M+14+H)⁺], 301.0 [(M+14+2+H)⁺].

### Step 5. Synthesis of 3-[2-bromo-5-formyl-4-(trifluoromethoxy)phenoxy]propanoic acid

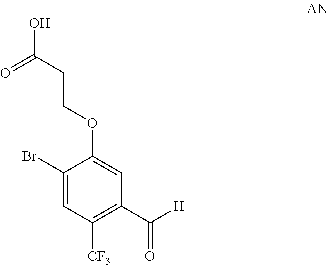 [SMILES: O=Cc1cc(OCCC(=O)O)c(Br)cc1C(F)(F)F]

To a solution of 4-bromo-5-hydroxy-2-(trifluoromethoxy)benzaldehyde (2.6 g, compound AN-5) in NaOH (9.1 mL, 1 M) were added 3-bromopropionic acid (1.4 g) with NaOH (9.1 mL, 1M). The resulting mixture was stirred at 110° C. for 16 hrs. The reaction mixture was acidified to pH=6 with 1 M HCl, extracted with EtOAc (80 mL) three times. The combined organic layer was washed by brine, dried over anhydrous sodium sulfate, concentrated in vacuo. The residue was purified by flash column chromatography (eluting with EtOAc/PE=1:8-1:1) to give 3-[2-bromo-5-formyl-4-(trifluoromethoxy)phenoxy]propanoic acid (1.0 g, Intermediate AN) as a light brown solid. MS obsd. (ESI⁺): 357.0 [(M+H)⁺], 359.0 [(M+2+H)⁺].

### Intermediate AO

### Methyl 3-[2-(p-tolylsulfonyloxy)ethoxy]cyclobutanecarboxylate

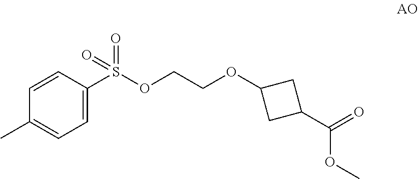

### Step 1: Preparation of 2-benzyloxyethoxy(trimethyl)silane

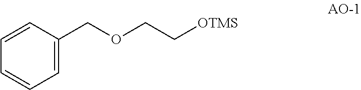

To a solution of 2-benzyloxyethanol (20.0 g, 131.4 mmol) and TEA (20.0 g, 197.1 mmol) in dichloromethane (200 mL) cooled at 0° C. was added trimethylsilyl chloride (17.1 g, 157.7 mmol) and the mixture was then stirred at 25° C. for 16 hrs. After the reaction was completed, the mixture was concentrated in vacuo and the residue was purified by column chromatography on silica gel (eluting with PE:EtOAc=50:1 to 10:1) to give the 2-benzyloxyethoxy(trimethyl)silane (25.0 g, 84.9%, compound AO-1) as a colorless oil.

### Step 2: Preparation of methyl 3-(2-benzyloxyethoxy)cyclobutanecarboxylate

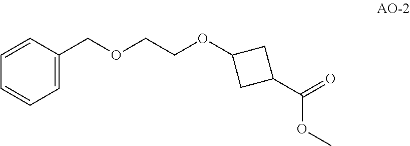 [SMILES: COC(=O)C1CC(OCCOCc2ccccc2)C1]

To a solution of 2-benzyloxyethoxy(trimethyl)silane (25.0 g, 111.4 mmol) and methyl 3-oxocyclobutanecarboxylate (CAS #: 4934-99-0, Cat. #: PB01390, from PharmaBlock (NanJing) R&D Co. Ltd, 15.0 g, 117.0 mmol) in dichloromethane (200 mL) was added trimethylsilyl trifluoromethanesulfonate (12.4 g, 55.7 mmol) dropwise at −78° C. After addition, the mixture was stirred at −78° C. for additional 1 hr, then to the resulting mixture was added triethylsilane (14.25 g, 122.57 mmol). After addition, the resulting mixture was warmed to room temperature and stirred for additional 1 hr. After the reaction was completed, the mixture was washed with saturated NH₄Cl solution, brine, dried over anhydrous sodium sulfate, and concentrated in vacuo. The residue was purified by column chromatography on silica gel (eluting with PE/EtOAc=100:1-50:1) to give methyl 3-(2-benzyloxyethoxy)cyclobutanecarboxylate (28 g, 95.1%, compound AO-2) as a colorless oil. MS obsd. (ESI⁺) [(M+H)⁺]: 265.1.

### Step 3: Preparation of methyl 3-(2-hydroxyethoxy)cyclobutanecarboxylate

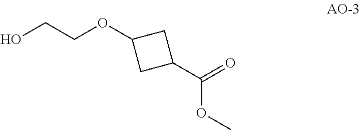

To a solution of methyl 3-(2-benzyloxyethoxy)cyclobutanecarboxylate (28.0 g, 105.9 mmol, compound AO-2) in MeOH (300.0 mL) was added Pd(OH)₂(wet) (1.48 g, 10.6 mmol) at room temperature and the mixture was then hydrogenated under H₂ atmosphere at room temperature overnight. After the reaction was completed, the reaction was filtered through silica gel pad and the filtrate was concentrated in vacuo to give 18 g crude methyl 3-(2-hydroxyethoxy)cyclobutanecarboxylate (18 g, 97.6%, compound AO-3) as a colorless oil.

### Step 4: Preparation of methyl 3-[2-(p-tolylsulfonyloxy)ethoxy]cyclobutanecarboxylate

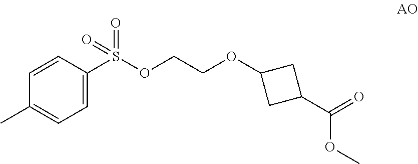

To a solution of methyl 3-(2-hydroxyethoxy)cyclobutanecarboxylate (5 g, 28.7 mmol) and DMAP (5.26 g, 43.1 mmol) in dichloromethane (80 mL) was added 4-methylbenzene-1-sulfonyl chloride (6.02 g, 31.6 mmol) at room temperature and the mixture was then stirred at room temperature overnight. After the reaction was completed, the mixture was washed with 1N HCl (25 mL), water (15 mL), saturated NaHCO₃ solution, brine and concentrated in vacuo to give the crude methyl 3-[2-(p-tolylsulfonyloxy)ethoxy]cyclobutanecarboxylate (8.1 g, 85.6%, Intermediate AO) as a colorless oil, which was used in the next step directly without further purification. MS obsd. (ESI⁺) [(M+H)⁺]: 329.2.

### Intermediate AP

### Cis-tert-butyl 3-[2-(p-tolylsulfonyloxy)ethoxy]cyclobutanecarboxylate

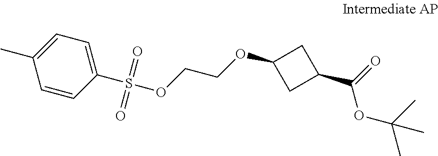 [SMILES: Cc1ccc(S(=O)(=O)OCCO[C@H]2C[C@@H](C(=O)OC(C)(C)C)C2)cc1]

### Step 1: preparation of cis-tert-butyl 3-(2-benzyloxyethoxy)cyclobutanecarboxylate

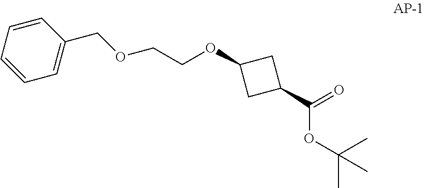 [SMILES: CC(C)(C)OC(=O)[C@H]1C[C@@H](OCCOCc2ccccc2)C1]

To a solution of trifluoromethanesulfonic anhydride (27.8 g, 98.56 mmol) and 2,6-lutidine (11.48 mL, 98.56 mmol) in DCM (100 mL) cooled at −30° C. was added 2-(benzyloxy)ethanol (10.0 g, 65.71 mmol) and the reaction mixture was stirred at −30° C. for 1 hr. The reaction mixture was washed with brine (30 ml) twice and the organic layer was concentrated in vacuo to give the crude 2-(benzyloxy)ethyltrifluoromethanesulfonate (18.7 g) as a yellow oil. To a solution of cis-tert-butyl 3-hydroxycyclobutanecarboxylate (CAS #: 939768-64-6, Cat. #: B253665, from BePharm Ltd., 11.3 g, 65.71 mmol) in THF (150 mL) cooled at 0° C. was added NaH (3.95 g, 98.56 mmol) and the mixture was stirred at room temperature for 1 hr. To the resulting solution was added 2-(benzyloxy)ethyltrifluoromethanesulfonate (18.7 g, previously prepared) and the mixture was stirred at room temperature for 2 hrs. The reaction was then quenched with ice water (100 mL) and extracted with EtOAc (200 mL) twice. The combined organic layer was dried over Na₂SO₄ and concentrated in vacuo. The residue was purified by column chromatography on silica gel (eluting with PE:EtOAc 100:1 to 2:1) to give cis-tert-butyl 3-(2-benzyloxyethoxy)cyclobutanecarboxylate (10.0 g, 49.67% yield) as a yellow oil. ¹H NMR (CDCl₃, 400 MHz): δ ppm 1.44 (s, 9H), 2.17 (m, 2H), 2.54-2.42 (m, 3H), 3.55-3.50 (m, 2H), 3.62-3.57 (m, 2H), 3.99-3.83 (m, 1H), 4.57 (s, 2H), 7.30-7.27 (m, 1H), 7.34 (d, J=4.3 Hz, 4H). MS obsd. (ESI⁺) [(M+Na)⁺]: 329.1.

### Step 2: preparation of cis-tert-butyl 3-[2-(p-tolylsulfonyloxy)ethoxy]cyclobutanecarboxylate

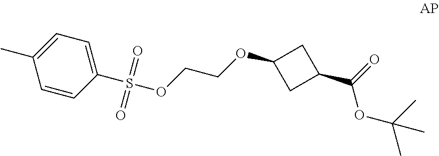 [SMILES: Cc1ccc(S(=O)(=O)OCCO[C@H]2C[C@@H](C(=O)OC(C)(C)C)C2)cc1]

Intermediate AP was prepared in analogy to the procedure described for the preparation of compound AN by using cis-tert-butyl 3-(2-benzyloxyethoxy)cyclobutanecarboxylate as the starting material instead of methyl 3-(2-benzyloxyethoxy)cyclobutanecarboxylate in Step 3. MS obsd. (ESI⁺) [(M+H)⁺]: 371.2.

### Intermediate AQ

### Tert-butyl 2-[3-(p-tolylsulfonyloxy)propoxy]acetate

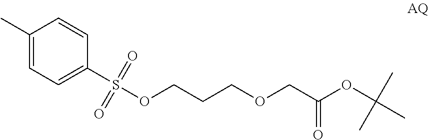 [SMILES: Cc1ccc(S(=O)(=O)OCCCOCC(=O)OC(C)(C)C)cc1]

### Step 1: Preparation of tert-butyl 2-(3-(benzyloxy)propoxy)acetate

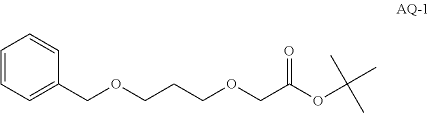 [SMILES: CC(C)(C)OC(=O)COCCCOCc1ccccc1]

To a mixture of NaOH (10M, 300.0 mL), tert-butyl 2-bromoacetate (23.5 g, 120.3 mmol) and tetrabutylammonium iodide (8.8 g, 24.06 mmol) in DCM (300 mL) was added 3-benzyloxypropan-1-ol (12.99 mL, 120.32 mmol) at 30° C. and the mixture was stirred at 30° C. for 72 hrs. After the reaction was completed, the organic phase was separated out and the aquatic phase was extracted with DCM (150 mL) twice. The combined organic layer was washed with brine, dried over MgSO₄ and concentrated in vacuo. The residue was purified by column chromatography on silica gel (eluting with PE:EtOAc=3:1) to give tert-butyl 2-(3-(benzyloxy)propoxy)acetate (21.3 g, 63.3% yield, compound AQ-1) as a colorless liquid. MS obsd. (ESI⁺) [(M+Na)⁺]: 303.2.

### Step 2: Preparation of tert-butyl 2-[3-(p-tolylsulfonyloxy)propoxy]acetate

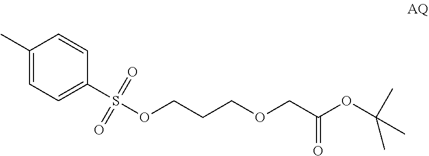 [SMILES: Cc1ccc(S(=O)(=O)OCCCOCC(=O)OC(C)(C)C)cc1]

AQ was prepared in analogy to the procedure described for the preparation of compound AN by using tert-butyl 2-(3-(benzyloxy)propoxy)acetate as the starting material instead of methyl 3-(2-benzyloxyethoxy)cyclobutanecarboxylate in Step 3. MS obsd. (ESI⁺) [(M+H)⁺]: 345.0.

### Example 1

### Cis-3-[2-[2-bromo-5-(8-chloro-4-oxo-chromen-2-yl)-4-methoxy-phenoxy]ethoxy]cyclobutanecarboxylic acid

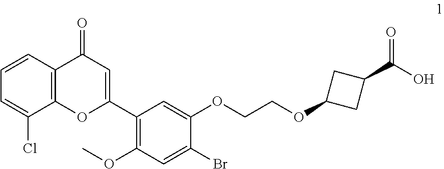 [SMILES: COc1cc(Br)c(OCCO[C@H]2C[C@@H](C(=O)O)C2)cc1-c1cc(=O)c2cccc(Cl)c2o1]

### Step 1: Synthesis of (E)-3-[4-bromo-2-methoxy-5-[(4-methoxyphenyl)methoxy]phenyl]-1-(3-chloro-2-hydroxy-phenyl)prop-2-en-1-one

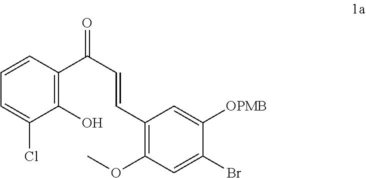

The mixture of 1-(3-chloro-2-hydroxy-phenyl)ethanone (0.39 g, 2.28 mmol), potassium hydroxide (0.51 g, 9.13 mmol) and 4-bromo-2-methoxy-5-[(4-methoxyphenyl)methoxy]benzaldehyde (0.8 g, Intermediate AA) in ethanol (40 mL) was stirred at 60° C. for 16 hrs. The reaction mixture was cooled to 0° C. and acidified to pH=3 with 4 M HCl solution. The precipitate was collected by filtration, washed with H₂O (50 mL), PE/EtOAc=50/1 (50 mL), dried in vacuo to give (E)-3-[4-bromo-2-methoxy-5-[(4-methoxyphenyl)methoxy]phenyl]-1-(3-chloro-2-hydroxy-phenyl)prop-2-en-1-one (800 mg, compound 1a) as a yellow solid which was used for next step without further purification.

### Step 2. Synthesis of 2-[4-bromo-2-methoxy-5-[(4-methoxyphenyl)methoxy]phenyl]-8-chloro-chromen-4-one

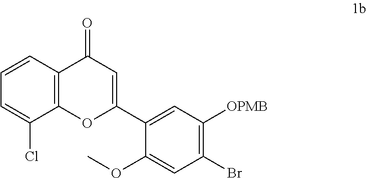

To a solution of (E)-3-[4-bromo-2-methoxy-5-[(4-methoxyphenyl)methoxy]phenyl]-1-(3-chloro-2-hydroxy-phenyl)prop-2-en-1-one (0.8 g, compound 1a) in DMSO (30 mL) was added iodine (48.37 mg). After being stirred at 140° C. for 4 hrs, the reaction mixture was cooled to 25° C. and poured into a mixture of Na₂SO₃ (18 mL) and ice/water (100 g). The precipitate was collected by filtration, washed with water (100 mL), dried in vacuo to give a crude yellow solid, which was purified by recrystallization with methanol/EtOAc (V/V)=1/40 (5 mL) to afford 2-[4-bromo-2-methoxy-5-[(4-methoxyphenyl)methoxy]phenyl]-8-chloro-chromen-4-one (900 mg, 90% yield, compound 1b) as a yellow solid.

### Step 3. Synthesis of 2-(4-bromo-5-hydroxy-2-methoxy-phenyl)-8-chloro-chromen-4-one

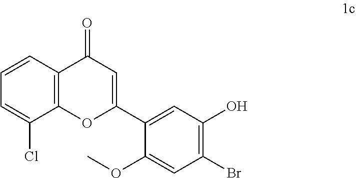 [SMILES: COc1cc(Br)c(O)cc1-c1cc(=O)c2cccc(Cl)c2o1]

To a solution of trifluoroacetic acid (7.5 mL) at 0° C. was added 2-[4-bromo-2-methoxy-5-[(4-methoxyphenyl)methoxy]phenyl]-8-chloro-chromen-4-one (425.0 mg, compound 1b). The reaction mixture was degassed with N₂ for 3 times. After being stirred at 0° C. for 4 hrs, the reaction mixture was concentrated in vacuo. The residue was purified by recrystallization with PE/EtOAc(V/V)=1/10 (30 mL) to afford 2-(4-bromo-5-hydroxy-2-methoxy-phenyl)-8-chloro-chromen-4-one (270 mg, 83% yield, compound 1c) as a brown solid. MS obsd. (ESI⁺): 381.0 [(M+H)⁺], 383.0 [(M+2+H)⁺].

### Step 4: Synthesis of tert-butyl 3-[2-[2-bromo-5-(8-chloro-4-oxo-chromen-2-yl)-4-methoxy-phenoxy]ethoxy]cyclobutanecarboxylate

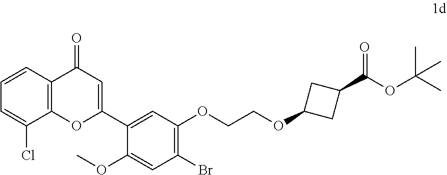 [SMILES: COc1cc(Br)c(OCCO[C@H]2C[C@@H](C(=O)OC(C)(C)C)C2)cc1-c1cc(=O)c2cccc(Cl)c2o1]

To a solution of 2-(4-bromo-5-hydroxy-2-methoxy-phenyl)-8-chloro-chromen-4-one (100.0 mg, 0.260 mmol, compound 1c) and cis-tert-butyl 3-[2-(p-tolylsulfonyloxy)ethoxy]cyclobutanecarboxylate (0.45 mL, Intermediate AP) in dry DMF (10 mL) was added potassium carbonate (72.44 mg). The reaction mixture was degassed with N₂ for 3 times. After being stirred at 100° C. for 16 hrs, the reaction mixture was concentrated in vacuo. The residue was diluted with EtOAc (50 mL), washed with brine (20 mL), dried over anhydrous sodium sulfate, filtered and concentrated in vacuo. The crude product was purified by flash silica gel column chromatography (eluting with EtOAc/PE=1/4) to give tert-butyl 3-[2-[2-bromo-5-(8-chloro-4-oxo-chromen-2-yl)-4-methoxy-phenoxy]ethoxy]cyclobutanecarboxylate (180 mg, 85% yield, compound 1d) as a yellow solid. MS obsd. (ESI⁺): 579.1 [(M+H)⁺], 581.1 [(M+2+H)⁺].

### Step 5: Synthesis of cis-3-[2-[2-bromo-5-(8-chloro-4-oxo-chromen-2-yl)-4-methoxy-phenoxy]ethoxy]-cyclobutanecarboxylic acid

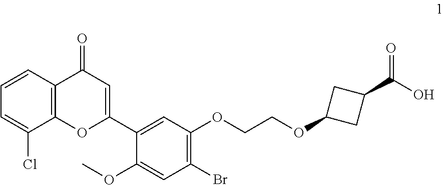 [SMILES: COc1cc(Br)c(OCCO[C@H]2C[C@@H](C(=O)O)C2)cc1-c1cc(=O)c2cccc(Cl)c2o1]

To a solution of cis-tert-butyl 3-[2-[2-bromo-5-(8-chloro-4-oxo-chromen-2-yl)-4-methoxy-phenoxy]ethoxy]cyclobutanecarboxylate (180.0 mg, compound 1d) in dry DCM (18 mL) was added trifluoroacetic acid (0.81 mL). The reaction mixture was degassed with N₂ for 3 times. After being stirred at 25° C. for 16 hrs, the reaction mixture was concentrated in vacuo. The residue was purified by re-crystallization with PE/EtOAc (V/V)=1/1 (30 mL) to give cis-3-[2-[2-bromo-5-(8-chloro-4-oxo-chromen-2-yl)-4-methoxy-phenoxy]ethoxy]cyclobutanecarboxylic acid (50 mg, Example 1) as a yellow solid. ¹H NMR (400 MHz, MeOD) δ ppm 8.04 (dd, J=8.0, 1.5 Hz, 1H), 7.89 (dd, J=7.8, 1.5 Hz, 1H), 7.74 (s, 1H), 7.40-7.48 (m, 2H), 7.27 (s, 1H), 4.17-4.24 (m, 2H), 4.04-4.12 (m, 1H), 3.96 (s, 3H), 3.77-3.83 (m, 2H), 2.60-2.68 (m, 1H), 2.50-2.58 (m, 2H), 2.13-2.2 (m, 2H). MS obsd. (ESI⁺): 523.0 [(M+H)⁺], 525.0 [(M+2+H)⁺].

### Example 2

### 3-[2-Bromo-5-(8-chloro-4-oxo-chromen-2-yl)-4-methoxy-phenoxy]propanoic acid

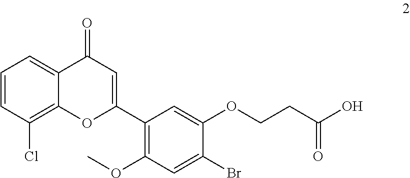 [SMILES: COc1cc(Br)c(OCCC(=O)O)cc1-c1cc(=O)c2cccc(Cl)c2o1]

To a solution of 2-(4-bromo-5-hydroxy-2-methoxy-phenyl)-8-chloro-chromen-4-one (100.0 mg, compound 1c) and beta-propiolactone (22.66 mg) in dry DMF (2 mL) was added, sodium hydride (7.55 mg) under N₂. After being stirred at 50° C. for 16 hrs, the reaction mixture was cooled to room temperature, and acidified to pH=6 with 2 M HCl at 0° C. The reaction mixture was concentrated in vacuo. The crude product was purified by prep-HPLC (eluting with ACN in water from 0% to 40%, 0.1% FA in water) to give 3-[2-bromo-5-(8-chloro-4-oxo-chromen-2-yl)-4-methoxy-phenoxy]propanoic acid (12 mg, Example 2) as a yellow solid. ¹H NMR (400 MHz, DMSO-d₆) δ ppm 7.95-8.03 (m, 2H), 7.68 (s, 1H), 7.45-7.49 (d, J=8.2 Hz, 2H), 7.08 (s, 1H), 4.26 (t, J=6.6 Hz, 2H), 3.93 (s, 3H), 2.58 (t, J=6.6 Hz, 2H). MS obsd. (ESI⁺): 453.0 [(M+H)⁺], 455.0 [(M+2+H)⁺].

### Example 3

### 3-[2-Bromo-5-(8-chloro-4-oxo-chromen-2-yl)-4-methoxy-phenoxy]-N-cyclopropylsulfonyl-propanamide

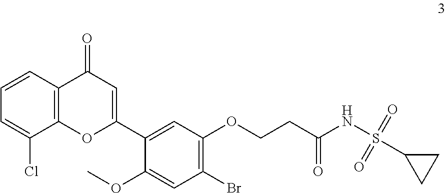 [SMILES: COc1cc(Br)c(OCCC(=O)NS(=O)(=O)C2CC2)cc1-c1cc(=O)c2cccc(Cl)c2o1]

### Step 1. Synthesis of methyl 3-[2-bromo-5-(8-chloro-4-oxo-chromen-2-yl)-4-methoxy-phenoxy]propanoate

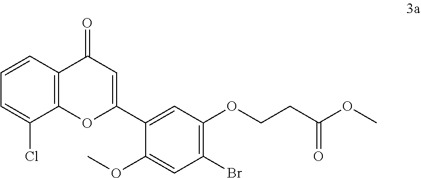 [SMILES: COC(=O)CCOc1cc(-c2cc(=O)c3cccc(Cl)c3o2)c(OC)cc1Br]

To a solution of 3-[2-bromo-5-(8-chloro-4-oxo-chromen-2-yl)-4-methoxy-phenoxy]propanoic acid (300.0 mg, example 2) in methanol (100 mL) at 0° C. was added thionyl chloride (236 mg) dropwise. After being stirred at 65° C. for 16 h the reaction mixture was concentrated in vacuo. The residue was diluted with water (50 mL), extracted with EtOAc (50 mL) three times. The combined organics were washed with brine (30 mL) two times, dried over anhydrous Na₂SO₄, filtered and concentrated in vacuo. The resulting oil was purified by flash silica gel column chromatography (eluting with EtOAc/PE=3/7) to give methyl 3-[2-bromo-5-(8-chloro-4-oxo-chromen-2-yl)-4-methoxy-phenoxy]propanoate (250 mg, 80% yield, compound 3a) as a yellow solid. MS obsd. (ESI⁺): 467.0 [(M+H)⁺], 469.0 [(M+2+H)⁺].

### Step 2. Synthesis of 3-[2-bromo-5-(8-chloro-4-oxo-chromen-2-yl)-4-methoxy-phenoxy]-N-cyclopropylsulfonyl-propanamide

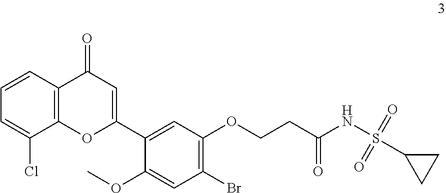 [SMILES: COc1cc(Br)c(OCCC(=O)NS(=O)(=O)C2CC2)cc1-c1cc(=O)c2cccc(Cl)c2o1]

To a solution of methyl 3-[2-bromo-5-(8-chloro-4-oxo-chromen-2-yl)-4-methoxy-phenoxy]propanoate (98.0 mg, 0.210 mmol, compound 3a) in 1,2-dichloroethane (10 mL) was added cyclopropanesulfonamide (0.05 g, 0.42 mmol) and titanium tetrachloride (0.12 g, 0.630 mmol) and stirred at 110° C. for 16 hrs. The reaction mixture was poured into ice/water (20 mL), extracted with DCM (15 mL) three times. The combined organic layer was dried over anhydrous sodium sulfate, filtered and concentrated in vacuo. The residue was purified by prep-HPLC (eluting with ACN in water 0% to 40%, 0.1% FA in water) to give 3-[2-bromo-5-(8-chloro-4-oxo-chromen-2-yl)-4-methoxy-phenoxy]-N-cyclopropylsulfonyl-propanamide (5 mg, 4.3% yield, Example 3) as a yellow solid. ¹H NMR (400 MHz, DMSO-d₆) δ ppm 8.31 (s, H), 8.00 (t, J=8.1 Hz, 2H), 7.71 (s, 1H), 7.48-7.53 (m, 2H), 7.10 (s, 1H), 4.27 (t, J=6.9 Hz, 2H), 3.94 (s, 3H), 2.69-2.82 (m, 2H), 2.50-2.55 (m, 1H), 0.63-0.83 (m, 4H). MS obsd. (ESI⁺): 556.0 [(M+H)⁺], 558.0 [(M+2+H)⁺].

### Example 4

### 2-[2-Bromo-5-(8-chloro-4-oxo-chromen-2-yl)-4-methoxy-phenoxy]acetic acid

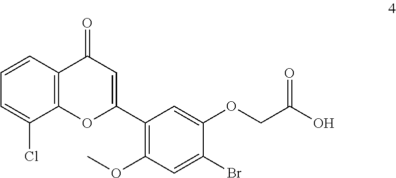 [SMILES: COc1cc(Br)c(OCC(=O)O)cc1-c1cc(=O)c2cccc(Cl)c2o1]

### Step 1. Synthesis of tert-butyl 2-[2-bromo-5-(8-chloro-4-oxo-chromen-2-yl)-4-methoxy-phenoxy]acetate

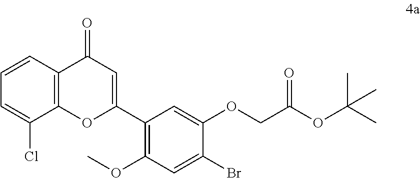 [SMILES: COc1cc(Br)c(OCC(=O)OC(C)(C)C)cc1-c1cc(=O)c2cccc(Cl)c2o1]

Compound 4a was prepared in analogy to the procedure described for the preparation of example 1 by using tert-butyl bromoacetate as the starting material instead of cis-tert-butyl 3-[2-(p-tolylsulfonyloxy)ethoxy]cyclobutanecarboxylate (Intermediate AO) in Step 4. Tert-butyl2-[2-bromo-5-(8-chloro-4-oxo-chromen-2-yl)-4-methoxy-phenoxy]acetate (70 mg, compound 4a) was obtained as a yellow solid. MS obsd. (ESI⁺): 495.0 [(M+H)⁺], 497.0 [(M+2+H)⁺].

### Step 2: Synthesis of 2-[2-bromo-5-(8-chloro-4-oxo-chromen-2-yl)-4-methoxy-phenoxy]acetic acid

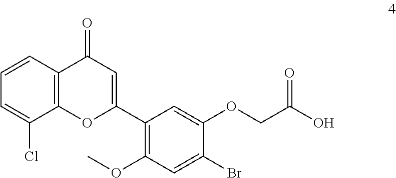 [SMILES: COc1cc(Br)c(OCC(=O)O)cc1-c1cc(=O)c2cccc(Cl)c2o1]

Example 4 was prepared in analogy to the procedure described for the preparation of example 1 by using tert-butyl 2-[2-bromo-5-(8-chloro-4-oxo-chromen-2-yl)-4-methoxy-phenoxy]acetate (compound 4a) as the starting material instead of tert-butyl 3-[2-[2-bromo-5-(8-chloro-4-oxo-chromen-2-yl)-4-methoxy-phenoxy]ethoxy]cyclobutanecarboxylate (compound 1d) in Step 5. 2-[2-Bromo-5-(8-chloro-4-oxo-chromen-2-yl)-4-methoxy-phenoxy]acetic acid (40 mg, 64% yield, Example 4) was obtained as a yellow solid. ¹H NMR (400 MHz, DMSO-d₆) δ ppm 13.15 (br s, 1H), 7.99 (t, J=8.0 Hz, 2H), 7.56 (s, 2H), 7.50 (t, J=7.9 Hz, 1H), 7.11 (s, 1H), 4.85 (s, 2H), 3.96 (s, 3H). MS obsd. (ESI⁺): 439.0 [(M+H)⁺], 441.0 [(M+2+H)⁺].

### Example 5

### 3-[2-Bromo-5-(8-chloro-4-oxo-chromen-2-yl)-4-methoxy-phenoxy]cyclobutanecarboxylic acid

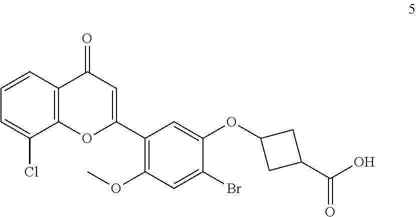 [SMILES: COc1cc(Br)c(OC2CC(C(=O)O)C2)cc1-c1cc(=O)c2cccc(Cl)c2o1]

### Step 1: Synthesis of tert-butyl 3-[2-bromo-5-(8-chloro-4-oxo-chromen-2-yl)-4-methoxy-phenoxy]cyclobutanecarboxylate

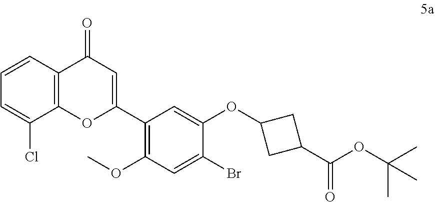 [SMILES: COc1cc(Br)c(OC2CC(C(=O)OC(C)(C)C)C2)cc1-c1cc(=O)c2cccc(Cl)c2o1]

Compound 5a was prepared in analogy to the procedure described for the preparation of example 1 by using as tert-butyl 3-(p-tolylsulfonyloxy)cyclobutanecarboxylate the starting material instead of cis-tert-butyl 3-[2-(p-tolylsulfonyloxy)ethoxy]cyclobutanecarboxylate (Intermediate AO) in Step 4. Tert-butyl 3-[2-bromo-5-(8-chloro-4-oxo-chromen-2-yl)-4-methoxy-phenoxy]cyclobutanecarboxylate (80 mg, 74% yield, compound 5a) was obtained as a yellow solid. MS obsd. (ESI⁺): 535.0 [(M+H)⁺], 537.1 [(M+2+H)⁺].

### Step 2. Synthesis of 3-[2-bromo-5-(8-chloro-4-oxo-chromen-2-yl)-4-methoxy-phenoxy]cyclobutanecarboxylic acid

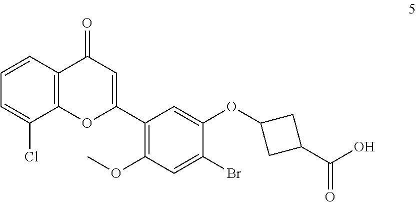 [SMILES: COc1cc(Br)c(OC2CC(C(=O)O)C2)cc1-c1cc(=O)c2cccc(Cl)c2o1]

Example 5 was prepared in analogy to the procedure described for the preparation of example 1 by using tert-butyl 3-[2-bromo-5-(8-chloro-4-oxo-chromen-2-yl)-4-methoxy-phenoxy]cyclobutanecarboxylate (compound 5a) as the starting material instead of tert-butyl 3-[2-[2-bromo-5-(8-chloro-4-oxo-chromen-2-yl)-4-methoxy-phenoxy]ethoxy]cyclobutanecarboxylate (compound 1d) in Step 5. 3-[2-Bromo-5-(8-chloro-4-oxo-chromen-2-yl)-4-methoxy-phenoxy]cyclobutanecarboxylic acid (23 mg, 35% yield, Example 5) was obtained as a yellow solid. ¹H NMR (400 MHz, DMSO-d₆) δ ppm 12.35 (br s, 1H), 7.97-8.03 (m, 2H), 7.54 (s, 2H), 7.49 (t, J=7.9 Hz, 1H), 7.17 (s, 1H), 4.91-4.98 (m, 1H), 3.95 (s, 3H), 3.063.13 (m, 1H), 2.67-2.76 (m, 2H), 2.33-2.46 (m, 2H). MS obsd. (ESI⁺): 479.0 [(M+H)⁺], 481.0 [(M+2+H)⁺].

### Example 6

### 2-[3-[2-Bromo-5-(8-chloro-4-oxo-chromen-2-yl)-4-methoxy-phenoxy]propoxy]acetic acid

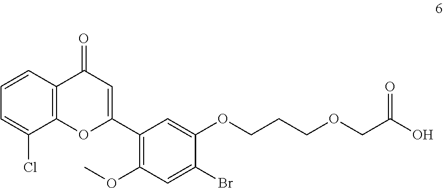 [SMILES: COc1cc(Br)c(OCCCOCC(=O)O)cc1-c1cc(=O)c2cccc(Cl)c2o1]

### Step 1. Synthesis of tert-butyl 2-[3-[2-bromo-5-(8-chloro-4-oxo-chromen-2-yl)-4-methoxy-phenoxy]propoxy]acetate

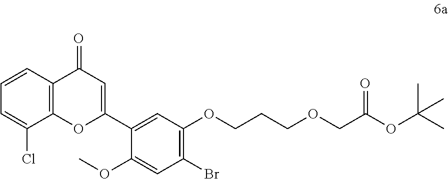 [SMILES: COc1cc(Br)c(OCCCOCC(=O)OC(C)(C)C)cc1-c1cc(=O)c2cccc(Cl)c2o1]

Compound 6a was prepared in analogy to the procedure described for the preparation of example 1 by using as tert-butyl 2-[3-(p-tolylsulfonyloxy)propoxy]acetate (Intermediate AQ) the starting material instead of cis-tert-butyl 3-[2-(p-tolylsulfonyloxy)ethoxy]cyclobutanecarboxylate (Intermediate AP) in Step 4. Tert-butyl 2-[3-[2-bromo-5-(8-chloro-4-oxo-chromen-2-yl)-4-methoxy-phenoxy]propoxy]acetate (180 mg, 59% yield, compound 6a) was obtained as a yellow solid. MS obsd. (ESI⁺): 553.0 [(M+H)⁺], 555.1 [(M+2+H)⁺].

### Step 2. Synthesis of 2-[3-[2-bromo-5-(8-chloro-4-oxo-chromen-2-yl)-4-methoxy-phenoxy]propoxy]acetic acid

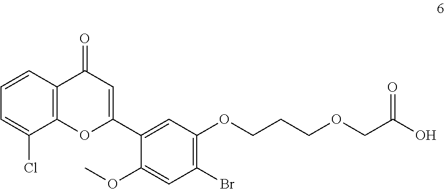 [SMILES: COc1cc(Br)c(OCCCOCC(=O)O)cc1-c1cc(=O)c2cccc(Cl)c2o1]

Example 6 was prepared in analogy to the procedure described for the preparation of example 1 by using tert-butyl 2-[3-[2-bromo-5-(8-chloro-4-oxo-chromen-2-yl)-4-methoxy-phenoxy]propoxy]acetate (compound 6a) as the starting material instead of tert-butyl 3-[2-[2-bromo-5-(8-chloro-4-oxo-chromen-2-yl)-4-methoxy-phenoxy]ethoxy]cyclobutanecarboxylate (compound 1d) in Step 5. 2-[3-[2-Bromo-5-(8-chloro-4-oxo-chromen-2-yl)-4-methoxy-phenoxy]propoxy]acetic acid (40 mg, 56% yield, Example 6) was obtained as a white solid. ¹H NMR (400 MHz, DMSO-d₆) δ ppm 12.51 (s, 1H), 7.98-8.06 (m, 2H), 7.65 (d, J=8.6 Hz, 1H), 7.58 (d, J=8.6 Hz, 1H), 7.52 (t, J=7.9 Hz, 1H), 6.93 (s, 1H), 4.11 (t, J=6.4 Hz, 2H), 4.03 (s, 2H), 3.93 (s, 3H), 3.70 (t, J=6.3 Hz, 2H), 2.05 (dd, J=13.0, 6.6 Hz, 2H). MS obsd. (ESI⁺): 497.0 [(M+H)⁺], 499.0 [(M+2+H)⁺].

### Example 7

### 3-[2-Chloro-5-(8-chloro-4-oxo-chromen-2-yl)-4-methoxy-phenoxy]cyclobutanecarboxylic acid

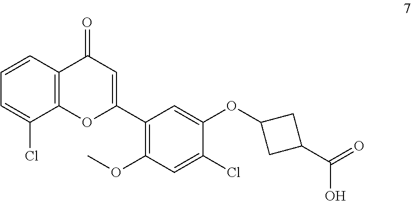 [SMILES: COc1cc(Cl)c(OC2CC(C(=O)O)C2)cc1-c1cc(=O)c2cccc(Cl)c2o1]

### Step 1. Synthesis of (E)-1-(3-chloro-2-hydroxy-phenyl)-3-[4-chloro-2-methoxy-5-(methoxymethoxy)phenyl]prop-2-en-1-one

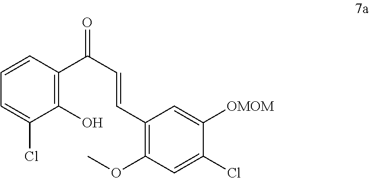

Compound 7a was prepared in analogy to the procedure described for the preparation of example 1 by using 4-chloro-2-methoxy-5-(methoxymethoxy)benzaldehyde (Intermediate AB) as the starting material instead of 4-bromo-2-methoxy-5-[(4-methoxyphenyl)methoxy]benzaldehyde (Intermediate AA) in Step 1. (E)-1-(3-chloro-2-hydroxy-phenyl)-3-[4-chloro-2-methoxy-5-(methoxymethoxy)phenyl]prop-2-en-1-one (280 mg, 70% yield, compound 7a) was obtained as a yellow solid which was used for next step without purification. MS obsd. (ESI⁺): 405.0 [(M+H)⁺], 407.0 [(M+2+H)⁺].

### Step 2. Synthesis of 8-chloro-2-(4-chloro-5-hydroxy-2-methoxy-phenyl)chromen-4-one

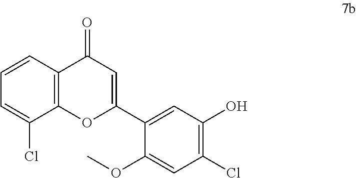 [SMILES: COc1cc(Cl)c(O)cc1-c1cc(=O)c2cccc(Cl)c2o1]

Compound 7b was prepared in analogy to the procedure described for the preparation of example 1 by using (E)-1-(3-chloro-2-hydroxy-phenyl)-3-[4-chloro-2-methoxy-5-(methoxymethoxy)phenyl]prop-2-en-1-one (280 mg, compound 7a) as the starting material instead of (E)-3-[4-bromo-2-methoxy-5-[(4-methoxyphenyl)methoxy]phenyl]-1-(3-chloro-2-hydroxy-phenyl)prop-2-en-1-one (compound 1a) in Step 2. 8-Chloro-2-(4-chloro-5-hydroxy-2-methoxy-phenyl)chromen-4-one (300 mg, 63% yield, compound 7b) was obtained as a brown solid. MS obsd. (ESI⁺): 337.0 [(M+H)⁺], 339.0 [(M+2+H)⁺].

### Step 3. Synthesis of methyl 3-[2-chloro-5-(8-chloro-4-oxo-chromen-2-yl)-4-methoxy-phenoxy]cyclobutanecarboxylate (7c)

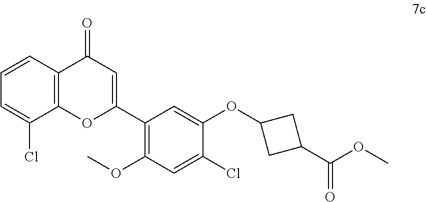 [SMILES: COC(=O)C1CC(Oc2cc(-c3cc(=O)c4cccc(Cl)c4o3)c(OC)cc2Cl)C1]

Compound 7c was prepared in analogy to the procedure described for the preparation of example 1 by using 8-chloro-2-(4-chloro-5-hydroxy-2-methoxy-phenyl)chromen-4-one (compound 7b) and methyl 3-(p-tolylsulfonyloxy)cyclobutanecarboxylate as the starting material instead of 2-(4-bromo-5-hydroxy-2-methoxy-phenyl)-8-chloro-chromen-4-one (compound 1c) and cis-tert-butyl 3-[2-(p-tolylsulfonyloxy)ethoxy]cyclobutanecarboxylate (Intermediate AP) in Step 4. Methyl 3-[2-chloro-5-(8-chloro-4-oxo-chromen-2-yl)-4-methoxy-phenoxy]cyclobutanecarboxylate (35 mg, 33% yield, compound 7c) was obtained as a white solid. MS obsd. (ESI⁺): 449.1 [(M+H)⁺], 451.1 [(M+2+H)⁺].

### Step 4. Synthesis of 3-[2-chloro-5-(8-chloro-4-oxo-chromen-2-yl)-4-methoxy-phenoxy]cyclobutanecarboxylic acid

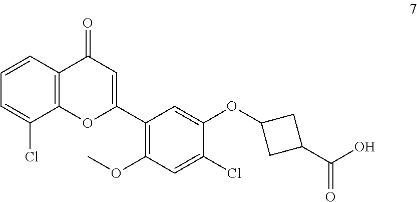 [SMILES: COc1cc(Cl)c(OC2CC(C(=O)O)C2)cc1-c1cc(=O)c2cccc(Cl)c2o1]

To a solution of methyl 3-[2-chloro-5-(8-chloro-4-oxo-chromen-2-yl)-4-methoxy-phenoxy]cyclobutanecarboxylate (35.0 mg, 0.080 mmol) in THF (10 mL) was added lithium hydroxide (0.01 mL, 0.780 mmol). The reaction mixture was degassed with N₂ for 3 times. After being stirred at 25° C. for 3 hrs, the reaction mixture was cooled to room temperature, and concentrated in vacuo. The residue was diluted with H₂O (5 mL), acidified to pH=6 with 2 M HCl at 0° C. The precipitate was collected, and washed with H₂O (5 mL), dried in vacuo. The crude product was purified by prep-HPLC (eluting with ACN in water 0% to 20%, 0.1% FA in water) to give 3-[2-chloro-5-(8-chloro-4-oxo-chromen-2-yl)-4-methoxy-phenoxy]cyclobutanecarboxylic acid (8 mg, 0.020 mmol, 23.59% yield, Example 7) as an off-white solid. ¹H NMR (400 MHz, DMSO-d₆) δ ppm 12.32 (br s, 1H), 7.97-8.02 (m, 2H), 7.59 (s, 1H), 7.50 (t, J=7.9 Hz, 1H), 7.43 (s, 1H), 7.17 (s, 1H), 4.92-4.99 (m, 1H), 3.95 (s, 1H), 3.10 (t, J=10.1 Hz, 1H), 2.70-2.80 (m, 2H), 2.40-2.47 (m, 2H). MS obsd. (ESI⁺): 435.1 [(M+H)⁺], 437.1 [(M+2+H)⁺].

### Example 8

### Cis-3-[2-[2-chloro-5-(8-chloro-4-oxo-chromen-2-yl)-4-methoxy-phenoxy]ethoxy]cyclobutanecarboxylic acid

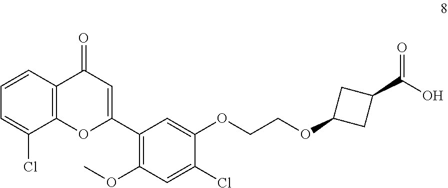 [SMILES: COc1cc(Cl)c(OCCO[C@H]2C[C@@H](C(=O)O)C2)cc1-c1cc(=O)c2cccc(Cl)c2o1]

### Step 1. Synthesis of cis-tert-butyl 3-[2-[2-chloro-5-(8-chloro-4-oxo-chromen-2-yl)-4-methoxy-phenoxy]ethoxy]cyclobutanecarboxylate

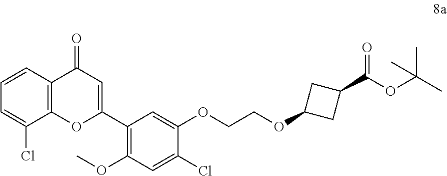 [SMILES: COc1cc(Cl)c(OCCO[C@H]2C[C@@H](C(=O)OC(C)(C)C)C2)cc1-c1cc(=O)c2cccc(Cl)c2o1]

Compound 8a was prepared in analogy to the procedure described for the preparation of example 1 by using 8-chloro-2-(4-chloro-5-hydroxy-2-methoxy-phenyl)chromen-4-one (compound 7b) as the starting material instead of 2-(4-bromo-5-hydroxy-2-methoxy-phenyl)-8-chloro-chromen-4-one (compound 1c) in Step 4. Cis-tert-butyl 3-[2-[2-chloro-5-(8-chloro-4-oxo-chromen-2-yl)-4-methoxy-phenoxy]ethoxy]cyclobutanecarboxylate (40 mg, 62% yield, compound 8a) was obtained as a white solid. MS obsd. (ESI⁺): 535.1 [(M+H)⁺], 537.2 [(M+2+H)⁺].

### Step 2. Synthesis of cis-3-[2-[2-chloro-5-(8-chloro-4-oxo-chromen-2-yl)-4-methoxy-phenoxy]ethoxy]cyclobutanecarboxylic acid

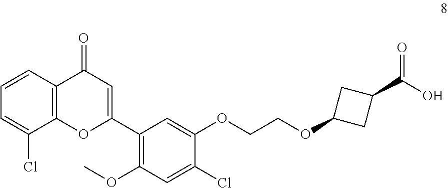 [SMILES: COc1cc(Cl)c(OCCO[C@H]2C[C@@H](C(=O)O)C2)cc1-c1cc(=O)c2cccc(Cl)c2o1]

Example 8 was prepared in analogy to the procedure described for the preparation of example 1 by using cis-tert-Butyl 3-[2-[2-chloro-5-(8-chloro-4-oxo-chromen-2-yl)-4-methoxy-phenoxy]ethoxy]cyclobutanecarboxylate (compound 8a) as the starting material instead of tert-butyl 3-[2-[2-bromo-5-(8-chloro-4-oxo-chromen-2-yl)-4-methoxy-phenoxy]ethoxy]cyclobutanecarboxylate (compound 1d) in Step 5. Cis-3-[2-[2-chloro-5-(8-chloro-4-oxo-chromen-2-yl)-4-methoxy-phenoxy]ethoxy]cyclobutanecarboxylic acid (26 mg, 72% yield, example 8) was obtained as a yellow solid. ¹H NMR (400 MHz, DMSO-d₆) δ ppm 7.89-7.97 (m, 2H), 7.62 (s, 1H) 7.42 (t, J=7.9 Hz, 1H), 7.34 (s, 1H), 7.00 (s, 1H), 4.15-4.09 (m, 2H), 3.89-3.96 (m, 1H), 3.88 (s, 3H), 3.61-3.66 (m, 2H), 2.49-2.54 (m, 1H), 2.32-2.40 (m, 2H), 1.88-1.97 (m, 2H). MS obsd. (ESI⁺): 479.1 [(M+H)⁺], 481.1 [(M+2+H)⁺].

### Example 9

### 3-[2-[5-(8-Chloro-4-oxo-chromen-2-yl)-4-methoxy-2-methyl-phenoxy]ethoxy]cyclobutanecarboxylic acid

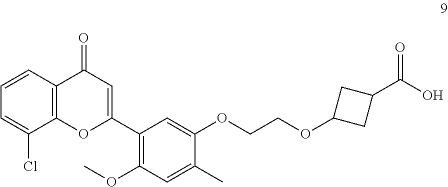 [SMILES: COc1cc(C)c(OCCOC2CC(C(=O)O)C2)cc1-c1cc(=O)c2cccc(Cl)c2o1]

### Step 1. Synthesis of tert-butyl 3-[2-[2-bromo-5-(8-chloro-4-oxo-chromen-2-yl)-4-methoxy-phenoxy]ethoxy]cyclobutanecarboxylate

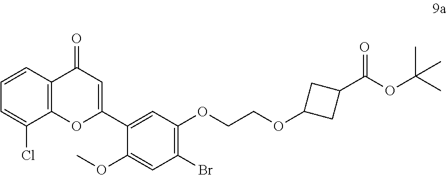 [SMILES: COc1cc(Br)c(OCCOC2CC(C(=O)OC(C)(C)C)C2)cc1-c1cc(=O)c2cccc(Cl)c2o1]

Compound 9a was prepared in analogy to the procedure described for the preparation of example 1 by using tert-butyl 3-[2-(p-tolylsulfonyloxy)ethoxy]cyclobutanecarboxylate as the starting material instead of cis-tert-butyl 3-[2-(p-tolylsulfonyloxy)ethoxy]cyclobutanecarboxylate (Intermediate AP) in Step 4. Tert-butyl-3-[2-[2-bromo-5-(8-chloro-4-oxo-chromen-2-yl)-4-methoxy-phenoxy]ethoxy]cyclobutanecarboxylate (180 mg, 79% yield, compound 9a) as a yellow solid. MS obsd. (ESI⁺): 579.1 [(M+H)⁺], 581.1 [(M+2+H)⁺].

### Step 2. Synthesis of tert-butyl 3-[2-[5-(8-chloro-4-oxo-chromen-2-yl)-4-methoxy-2-methyl-phenoxy]ethoxy]cyclobutanecarboxylate

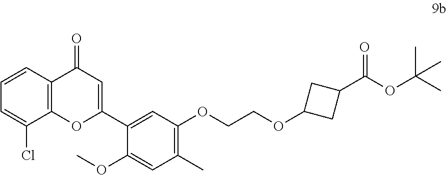 [SMILES: COc1cc(C)c(OCCOC2CC(C(=O)OC(C)(C)C)C2)cc1-c1cc(=O)c2cccc(Cl)c2o1]

To a solution of tert-butyl 3-[2-[2-bromo-5-(8-chloro-4-oxo-chromen-2-yl)-4-methoxy-phenoxy]ethoxy]cyclobutanecarboxylate (180.0 mg, compound 9a) in 1,4-dioxane (15 mL) were added cesium carbonate (202.28 mg) and [1,1′-bis(diphenylphosphino)ferrocene]dichloropalladium(II) (22 mg). After addition, the mixture was degassed with N₂ for 3 times. Then trimethylboroxine (39 mg) was added. After being stirred at 100° C. for 8 hrs, the reaction mixture was concentrated in vacuo. The residue was diluted with ethyl acetate (50 mL), washed with brine (20 mL), dried over anhydrous sodium sulfate, filtered and concentrated in vacuo. The residue was purified by flash silica gel chromatography (eluting with EtOAc/PE=1/3) to give tert-butyl 3-[2-[5-(8-chloro-4-oxo-chromen-2-yl)-4-methoxy-2-methyl-phenoxy]ethoxy]cyclobutanecarboxylate (130 mg, 74% yield, compound 9b) as a yellow solid. MS obsd. (ESI⁺): 515.2 [(M+H)⁺], 517.2 [(M+2+H)⁺].

### Step 3. Synthesis of 3-[2-[5-(8-chloro-4-oxo-chromen-2-yl)-4-methoxy-2-methyl-phenoxy]ethoxy]cyclobutanecarboxylic acid

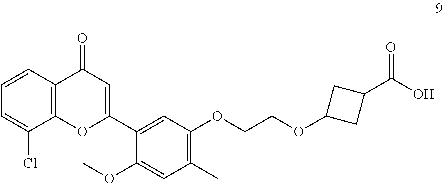 [SMILES: COc1cc(C)c(OCCOC2CC(C(=O)O)C2)cc1-c1cc(=O)c2cccc(Cl)c2o1]

Example 9 was prepared in analogy to the procedure described for the preparation of example 1 by using tert-butyl 3-[2-[5-(8-chloro-4-oxo-chromen-2-yl)-4-methoxy-2-methyl-phenoxy]ethoxy]cyclobutanecarboxylate (compound 9a) as the starting material instead of 2-(4-bromo-5-hydroxy-2-methoxy-phenyl)-8-chloro-chromen-4-one (compound 1c) in Step 4. 3-[2-[5-(8-Chloro-4-oxo-chromen-2-yl)-4-methoxy-2-methyl-phenoxy]ethoxy]cyclobutanecarboxylic acid (50 mg, 47% yield, example 9) was obtained as a yellow solid. ¹H NMR (400 MHz, DMSO-d₆) δ ppm 12.16 (s, 1H), 7.95-8.03 (m, 2H), 7.53 (d, J=2.8 Hz, 1H), 7.48 (td, J=7.9, 2.1 Hz, 1H), 7.15 (d, J=3.6 Hz, 1H), 7.09 (d, J=1.2 Hz, 1H), 4.19-4.22 (m, 0.25H), 4.10-4.14 (m, 2H), 3.95-4.00 (m, 0.75H), 3.92 (s, 3H), 3.64-3.73 (m, 2H), 2.85-2.95 (m, 0.25H), 2.55-2.63 (m, 0.75H), 2.38-2.48 (m, 2H), 2.28 (s, 3H), 2.15-2.20 (m, 0.5H), 1.95-2.04 (m, 1.5H). MS obsd. (ESI⁺): 459.1 [(M+H)⁺], 461.1 [(M+2+H)⁺].

### Example 10

### 3-[5-(8-Chloro-4-oxo-chromen-2-yl)-4-methoxy-2-methyl-phenoxy]propanoic acid

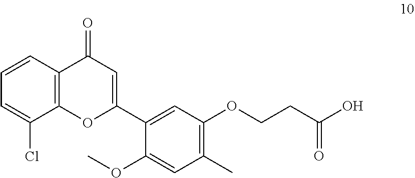 [SMILES: COc1cc(C)c(OCCC(=O)O)cc1-c1cc(=O)c2cccc(Cl)c2o1]

### Step 1. Synthesis of methyl 3-[5-(8-chloro-4-oxo-chromen-2-yl)-4-methoxy-2-methyl-phenoxy]propanoate

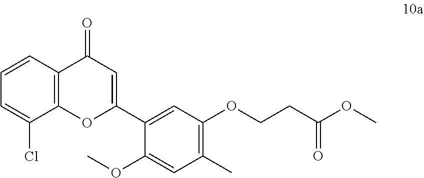 [SMILES: COC(=O)CCOc1cc(-c2cc(=O)c3cccc(Cl)c3o2)c(OC)cc1C]

Compound 10a was prepared in analogy to the procedure described for the preparation of example 9 by using methyl 3-[2-bromo-5-(8-chloro-4-oxo-chromen-2-yl)-4-methoxy-phenoxy]propanoate (compound 3a) as the starting material instead of tert-butyl 3-[2-[2-bromo-5-(8-chloro-4-oxo-chromen-2-yl)-4-methoxy-phenoxy]ethoxy]cyclobutanecarboxylate (compound 9a) in Step 2. Methyl 3-[5-(8-chloro-4-oxo-chromen-2-yl)-4-methoxy-2-methyl-phenoxy]propanoate (80 mg, 79% yield, compound 10a) was obtained as a yellow solid, which was used for next step without further purification. MS obsd. (ESI⁺): 403.0 [(M+H)⁺], 405.1 [(M+2+H)⁺].

### Step 2. Synthesis of 3-[5-(8-chloro-4-oxo-chromen-2-yl)-4-methoxy-2-methyl-phenoxy]propanoic acid

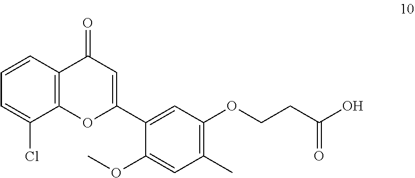 [SMILES: COc1cc(C)c(OCCC(=O)O)cc1-c1cc(=O)c2cccc(Cl)c2o1]

Example 10 was prepared in analogy to the procedure described for the preparation of example 7 by using methyl 3-[5-(8-chloro-4-oxo-chromen-2-yl)-4-methoxy-2-methyl-phenoxy]propanoate (80 mg, compound 10a) as the starting material instead of methyl 3-[2-chloro-5-(8-chloro-4-oxo-chromen-2-yl)-4-methoxy-phenoxy]cyclobutanecarboxylate (compound 7c) in Step 4. 3-[5-(8-Chloro-4-oxo-chromen-2-yl)-4-methoxy-2-methyl-phenoxy]propanoic acid (3.5 mg, 5.3% yield, Example 10) was obtained as a yellow solid. ¹H NMR (400 MHz, DMSO-d₆) δ ppm 7.96 (d, J=7.6 Hz, 2H), 7.55 (s, 1H), 7.46 (t, J=7.7 Hz, 1H), 7.09 (s, 2H), 4.21 (t, J=6.0 Hz, 2H), 3.90 (s, 3H), 2.61 (t, J=6.0 Hz, 2H), 2.21 (s, 3H). MS obsd. (ESI⁺): 389.0 [(M+H)⁺], 391.1 [(M+2+H)⁺].

### Example 11

### 2-[3-[5-(8-Chloro-4-oxo-chromen-2-yl)-4-methoxy-2-methyl-phenoxy]propoxy]acetic acid

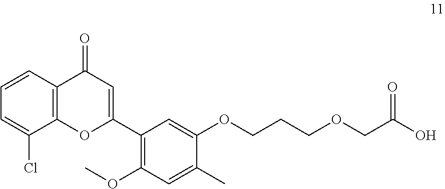 [SMILES: COc1cc(C)c(OCCCOCC(=O)O)cc1-c1cc(=O)c2cccc(Cl)c2o1]

### Step 1. Synthesis of tert-butyl 2-[3-[5-(8-chloro-4-oxo-chromen-2-yl)-4-methoxy-2-methyl-phenoxy]propoxy]acetate

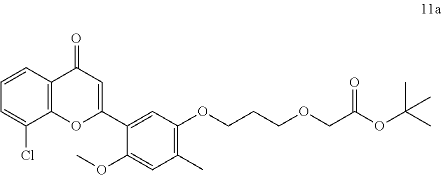 [SMILES: COc1cc(C)c(OCCCOCC(=O)OC(C)(C)C)cc1-c1cc(=O)c2cccc(Cl)c2o1]

Compound 11a was prepared in analogy to the procedure described for the preparation of example 9 by using tert-butyl 2-[3-[2-bromo-5-(8-chloro-4-oxo-chromen-2-yl)-4-methoxy-phenoxy]propoxy]acetate (compound 6a) as the starting material instead of tert-butyl 3-[2-[2-bromo-5-(8-chloro-4-oxo-chromen-2-yl)-4-methoxy-phenoxy]ethoxy]cyclobutanecarboxylate (compound 9a) in Step 2. Tert-butyl 2-[3-[5-(8-chloro-4-oxo-chromen-2-yl)-4-methoxy-2-methyl-phenoxy]propoxy]acetate (80 mg, 81% yield, Example 11) was obtained as a yellow solid. MS obsd. (ESI⁺): 489.2 [(M+H)⁺], 491.2 [(M+2+H)⁺].

### Step 2. Synthesis of 2-[3-[5-(8-chloro-4-oxo-chromen-2-yl)-4-methoxy-2-methyl-phenoxy]propoxy]acetic acid

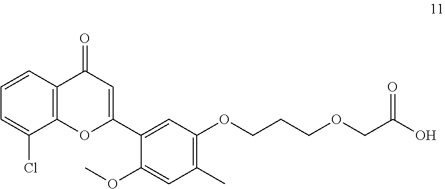 [SMILES: COc1cc(C)c(OCCCOCC(=O)O)cc1-c1cc(=O)c2cccc(Cl)c2o1]

Example 11 was prepared in analogy to the procedure described for the preparation of example 1 by using tert-butyl 2-[3-[5-(8-chloro-4-oxo-chromen-2-yl)-4-methoxy-2-methyl-phenoxy]propoxy]acetate (compound 11a) as the starting material instead of tert-butyl 3-[2-[2-bromo-5-(8-chloro-4-oxo-chromen-2-yl)-4-methoxy-phenoxy]ethoxy]cyclobutanecarboxylate (compound 1d) in Step 5. 2-[3-[5-(8-Chloro-4-oxo-chromen-2-yl)-4-methoxy-2-methyl-phenoxy]propoxy]acetic acid (20 mg, 35% yield, Example 11) was obtained as a white solid. ¹H NMR (400 MHz, DMSO-d₆) δ ppm 12.59 (s, 1H), 7.95-8.03 (m, 2H), 7.55 (s, 1H), 7.48 (t, J=7.9 Hz, 1H), 7.14 (s, 1H), 7.10 (s, 1H), 4.11 (t, J=5.9 Hz, 2H), 4.02 (s, 2H), 3.93 (d, J=10.8 Hz, 2H), 3.66 (s, 1H), 2.26 (s, 3H), 1.98-2.07 (m, 2H). MS obsd. (ESI⁺): 433.1 [(M+H)⁺], 435.1 [(M+2+H)⁺].

### Example 12

### 3-[5-(8-Chloro-4-oxo-chromen-2-yl)-2,4-dimethoxy-phenoxy]cyclobutanecarboxylic acid

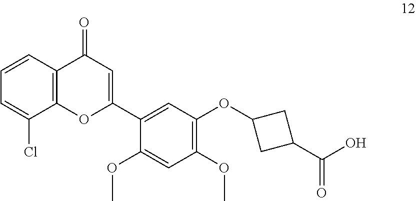 [SMILES: COc1cc(OC)c(-c2cc(=O)c3cccc(Cl)c3o2)cc1OC1CC(C(=O)O)C1]

### Step 1. Synthesis of (E)-1-(3-chloro-2-hydroxy-phenyl)-3-(5-hydroxy-2,4-dimethoxy-phenyl)prop-2-en-1-one

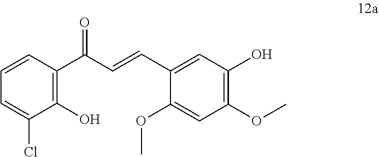 [SMILES: COc1cc(OC)c(/C=C/C(=O)c2cccc(Cl)c2O)cc1O]

Compound 12a was prepared in analogy to the procedure described for the preparation of example 1 by using 5-Hydroxy-2,4-dimethoxy-benzaldehyde (Intermediate AC) as the starting material instead of 4-bromo-2-methoxy-5-[(4-methoxyphenyl)methoxy]benzaldehyde (Intermediate AA) in Step 1. (E)-1-(3-chloro-2-hydroxy-phenyl)-3-(5-hydroxy-2,4-dimethoxy-phenyl)prop-2-en-1-one (4 g, 87%, yield, compound 12a) was obtained as a yellow solid, which was used in the next step directly without further purification. MS obsd. (ESI⁺) [(M+H)⁺]: 335.

### Step 2. Synthesis of 8-chloro-2-(5-hydroxy-2,4-dimethoxy-phenyl)chromen-4-one

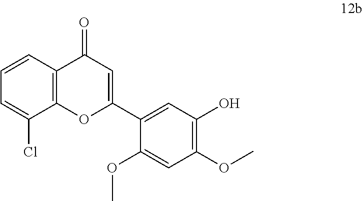 [SMILES: COc1cc(OC)c(-c2cc(=O)c3cccc(Cl)c3o2)cc1O]

Compound 12b was prepared in analogy to the procedure described for the preparation of example 1 by using (E)-1-(3-chloro-2-hydroxyphenyl)-3-(5-hydroxy-2,4-dimethoxyphenyl)prop-2-en-1-one (500 mg, compound 12a) as the starting material instead of (E)-3-[4-bromo-2-methoxy-5-[(4-methoxyphenyl)methoxy]phenyl]-1-(3-chloro-2-hydroxy-phenyl)prop-2-en-1-one (compound 1a) in Step 2. 8-Chloro-2-(5-hydroxy-2,4-dimethoxy-phenyl)chromen-4-one (400 mg, yield 85%, compound 12b) was obtained as light yellow solid. MS obsd. (ESI⁺) [(M+H)⁺]: 333.

### Step 3. Synthesis of methyl 3-[5-(8-chloro-4-oxo-chromen-2-yl)-2,4-dimethoxy-phenoxy]cyclobutanecarboxylate (12c)

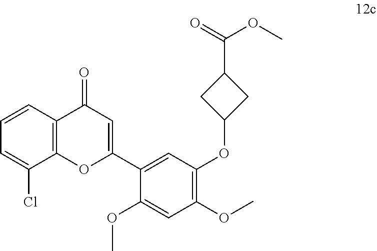 [SMILES: COC(=O)C1CC(Oc2cc(-c3cc(=O)c4cccc(Cl)c4o3)c(OC)cc2OC)C1]

Compound 12c was prepared in analogy to the procedure described for the preparation of example 1 by using 8-chloro-2-(5-hydroxy-2,4-dimethoxy-phenyl)chromen-4-one (compound 12b) and methyl 3-(p-tolylsulfonyloxy)cyclobutanecarboxylate as the starting material instead of 2-(4-bromo-5-hydroxy-2-methoxy-phenyl)-8-chloro-chromen-4-one (compound 1c) and cis-tert-butyl 3-[2-(p-tolylsulfonyloxy)ethoxy]cyclobutanecarboxylate (Intermediate AP) in Step 4. Methyl 3-[5-(8-chloro-4-oxo-chromen-2-yl)-2,4-dimethoxy-phenoxy]cyclobutanecarboxylate (200 mg, 74.8% yield, compound 12c) was obtained as a yellow oil and was used in the next step directly. MS obsd. (ESI⁺) [(M+H)⁺]: 445.

### Step 4. Synthesis of 3-[5-(8-Chloro-4-oxo-chromen-2-yl)-2,4-dimethoxy-phenoxy]cyclobutanecarboxylic acid

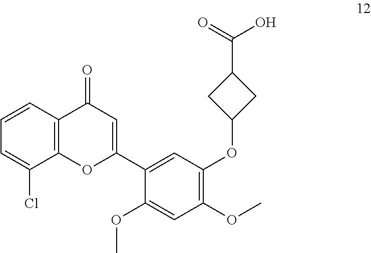 [SMILES: COc1cc(OC)c(-c2cc(=O)c3cccc(Cl)c3o2)cc1OC1CC(C(=O)O)C1]

Example 12 was prepared in analogy to the procedure described for the preparation of example 7 by using methyl 3-[5-(8-chloro-4-oxo-chromen-2-yl)-2,4-dimethoxy-phenoxy]cyclobutanecarboxylate (compound 12c) as the starting material instead of methyl 3-[2-chloro-5-(8-chloro-4-oxo-chromen-2-yl)-4-methoxy-phenoxy]cyclobutanecarboxylate (compound 7c) in Step 4. 3-[5-(8-Chloro-4-oxo-chromen-2-yl)-2,4-dimethoxy-phenoxy]cyclobutanecarboxylic acid (40 mg, 41% yield, example 12) was obtained as a yellow powder. ¹H NMR (400 MHz, DMSO-d₆) δ ppm 12.2-12.4 (m, 1H), 7.9-8.0 (m, 2H), 7.4-7.5 (m, 2H), 7.1-7.2 (m, 1H), 6.9-6.9 (m, 1H), 4.9-4.6 (m, 1H), 4.0-4.0 (m, 3H), 3.93 (s, 3H), 3.06-2.8 (m, 1H), 2.7-2.8 (m, 2H), 2.3-2.5 (m, 1H), 2.2-2.3 (m, 1H). MS obsd. (ESI⁺) [(M+H)⁺]: 431.

### Example 13

### 3-[2-[5-(8-Chloro-4-oxo-chromen-2-yl)-2,4-dimethoxy-phenoxy]ethoxy]cyclobutanecarboxylic acid

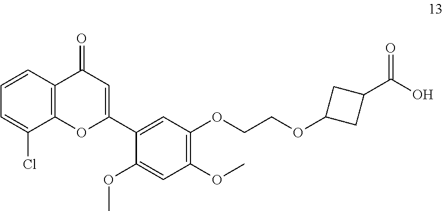 [SMILES: COc1cc(OC)c(-c2cc(=O)c3cccc(Cl)c3o2)cc1OCCOC1CC(C(=O)O)C1]

### Step 1. Synthesis of methyl 3-[2-[5-(8-chloro-4-oxo-chromen-2-yl)-2,4-dimethoxy-phenoxy]ethoxy]cyclobutanecarboxylate

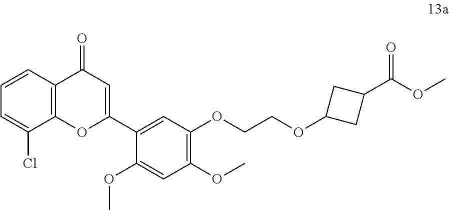 [SMILES: COC(=O)C1CC(OCCOc2cc(-c3cc(=O)c4cccc(Cl)c4o3)c(OC)cc2OC)C1]

Compound 13a was prepared in analogy to the procedure described for the preparation of example 1 by using 8-chloro-2-(5-hydroxy-2,4-dimethoxy-phenyl)chromen-4-one (compound 12b) and methyl 3-[2-(p-tolylsulfonyloxy)ethoxy]cyclobutanecarboxylate (Intermediate AO) as the starting material instead of 2-(4-bromo-5-hydroxy-2-methoxy-phenyl)-8-chloro-chromen-4-one (compound 1c) and cis-tert-butyl 3-[2-(p-tolylsulfonyloxy)ethoxy]cyclobutanecarboxylate in Step 4. Methyl 3-[2-[5-(8-chloro-4-oxo-chromen-2-yl)-2,4-dimethoxy-phenoxy]ethoxy]cyclobutanecarboxylate (200 mg, 74.8% yield, compound 13a) was obtained as a yellow oil and the residue was used in the next step directly. MS obsd. (ESI⁺) [(M+H)⁺]: 445.

### Step 2. Synthesis of 3-[2-[5-(8-chloro-4-oxo-chromen-2-yl)-2,4-dimethoxy-phenoxy]ethoxy]cyclobutanecarboxylic acid

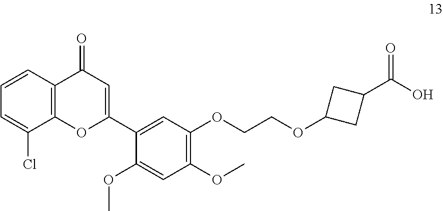 [SMILES: COc1cc(OC)c(-c2cc(=O)c3cccc(Cl)c3o2)cc1OCCOC1CC(C(=O)O)C1]

Example 13 was prepared in analogy to the procedure described for the preparation of example 7 by using methyl 3-[2-[5-(8-chloro-4-oxo-chromen-2-yl)-2,4-dimethoxy-phenoxy]ethoxy]cyclobutanecarboxylate (compound 13a) as the starting material instead of methyl 3-[2-chloro-5-(8-chloro-4-oxo-chromen-2-yl)-4-methoxy-phenoxy]cyclobutanecarboxylate (compound 7c) in Step 4. 3-[2-[5-(8-Chloro-4-oxo-chromen-2-yl)-2,4-dimethoxy-phenoxy]ethoxy]cyclobutanecarboxylic acid (80 mg, 39.9% yield, Example 13) was obtained as a yellow solid. ¹H NMR (400 MHz, DMSO-d₆) δ ppm 12.0-12.3 (m, 1H), 7.9-8.0 (m, 2H), 7.6-7.6 (m, 1H), 7.4-7.5 (m, 1H), 7.08 (s, 1H), 6.9-6.9 (m, 1H), 4.1-4.2 (m, 1H), 4.09 (br s, 2H), 3.99 (s, 3H), 3.94 (s, 3H), 3.65 (br d, 2H, J=1.1 Hz), 2.8-3.0 (m, 1H), 2.3-2.5 (m, 2H), 2.1-2.2 (m, 1H), 1.9-2.0 (m, 1H). MS obsd. (ESI⁺) [(M+H)⁺]: 475.

### Example 14

### 2-[5-(8-Chloro-4-oxo-chromen-2-yl)-2-methyl-phenoxy]acetic acid

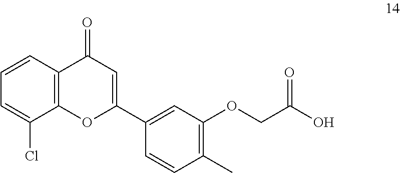 [SMILES: Cc1ccc(-c2cc(=O)c3cccc(Cl)c3o2)cc1OCC(=O)O]

### Step 1. Synthesis of ethyl 2-[5-(8-chloro-4-oxo-chromen-2-yl)-2-methyl-phenoxy]acetate

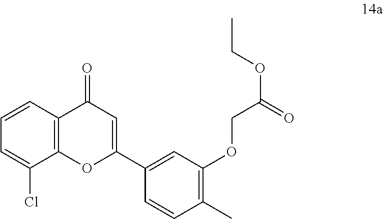 [SMILES: CCOC(=O)COc1cc(-c2cc(=O)c3cccc(Cl)c3o2)ccc1C]

Compound 14a was prepared in analogy to the procedure described for the preparation of example 1 by using 8-chloro-2-(3-hydroxy-4-methyl-phenyl)chromen-4-one and ethyl 2-bromoacetate as the starting material instead of 2-(4-bromo-5-hydroxy-2-methoxy-phenyl)-8-chloro-chromen-4-one (compound 1c) and cis-tert-butyl 3-[2-(p-tolylsulfonyloxy)ethoxy]cyclobutanecarboxylate in Step 4. Ethyl 2-(5-(8-chloro-4-oxo-chromen-2-yl)-2-methylphenoxy)acetate (240 mg, 92.3% yield, compound 14a) was obtained as a yellow solid, which was used in the next step directly. MS obsd. (ESI⁺) [(M+H)⁺]: 373.

### Step 2. Synthesis of 2-[5-(8-chloro-4-oxo-chromen-2-yl)-2-methyl-phenoxy]acetic acid

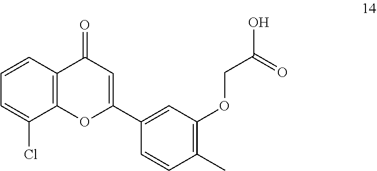 [SMILES: Cc1ccc(-c2cc(=O)c3cccc(Cl)c3o2)cc1OCC(=O)O]

Example 14 was prepared in analogy to the procedure described for the preparation of example 7 by using ethyl 2-(5-(8-chloro-4-oxo-chromen-2-yl)-2-methylphenoxy)acetate (compound 14a) as the starting material instead of methyl 3-[2-chloro-5-(8-chloro-4-oxo-chromen-2-yl)-4-methoxy-phenoxy]cyclobutanecarboxylate (compound 7c) in Step 4. 2-[5-(8-Chloro-4-oxo-chromen-2-yl)-2-methyl-phenoxy]acetic acid (23 mg, 11.9% yield, example 14) was obtained as a yellow foam. ¹H NMR (400 MHz, DMSO-d₆) δ ppm 13.07 (br s, 1H), 8.0-8.0 (m, 2H), 7.67 (dd, 1H, J=1.5, 7.8 Hz), 7.5-7.6 (m, 2H), 7.41 (d, 1H, J=8.3 Hz), 7.22 (s, 1H), 4.89 (s, 2H), 2.29 (s, 3H). MS obsd. (ESI⁺) [(M+H)⁺]: 345.

### Example 15 and Example 16

### Cis-3-[5-(8-chloro-4-oxo-chromen-2-yl)-2-methyl-phenoxy]cyclobutanecarboxylic acid (Example 15) and trans-3-[5-(8-chloro-4-oxo-chromen-2-yl)-2-methyl-phenoxy]cyclobutanecarboxylic acid (Example 16)

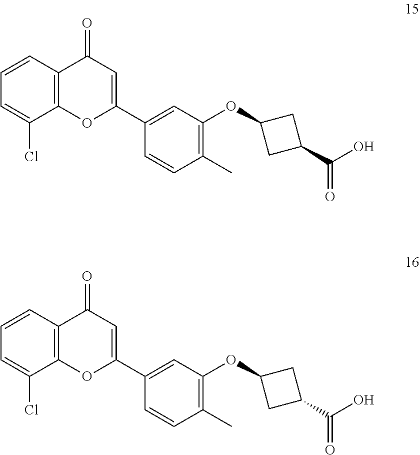 [SMILES: Cc1ccc(-c2cc(=O)c3cccc(Cl)c3o2)cc1O[C@H]1C[C@@H](C(=O)O)C1 ; Cc1ccc(-c2cc(=O)c3cccc(Cl)c3o2)cc1O[C@H]1C[C@H](C(=O)O)C1]

### Step 1. Synthesis of methyl 3-[5-(8-chloro-4-oxo-chromen-2-yl)-2-methyl-phenoxy]cyclobutanecarboxylate

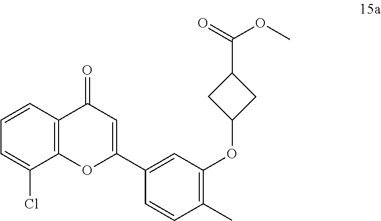 [SMILES: COC(=O)C1CC(Oc2cc(-c3cc(=O)c4cccc(Cl)c4o3)ccc2C)C1]

Compound 15a was prepared in analogy to the procedure described for the preparation of example 1 by using 8-chloro-2-(3-hydroxy-4-methyl-phenyl)chromen-4-one and methyl 3-chlorocyclobutane-1-carboxylate as the starting material instead of 2-(4-bromo-5-hydroxy-2-methoxy-phenyl)-8-chloro-chromen-4-one (compound 1c) and tert-butyl 3-[2-(p-tolylsulfonyloxy)ethoxy]cyclobutanecarboxylate in Step 4. Methyl 3-[5-(8-chloro-4-oxo-chromen-2-yl)-2-methyl-phenoxy]cyclobutanecarboxylate (250 mg, 89% yield, compound 15a) was obtained as a yellow oil, which was used in the next step directly. MS obsd. (ESI⁺) [(M+H)⁺]: 399.

### Step 2. Synthesis of 3-[5-(8-chloro-4-oxo-chromen-2-yl)-2-methyl-phenoxy]cyclobutanecarboxylic acid

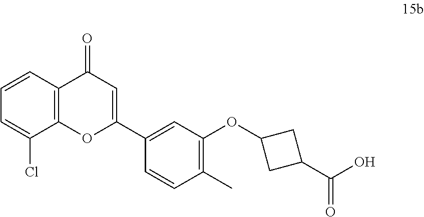 [SMILES: Cc1ccc(-c2cc(=O)c3cccc(Cl)c3o2)cc1OC1CC(C(=O)O)C1]

Compound 15b was prepared in analogy to the procedure described for the preparation of example 7 by using methyl 3-[5-(8-chloro-4-oxo-chromen-2-yl)-2-methyl-phenoxy]cyclobutanecarboxylate (compound 15a) as the starting material instead of methyl 3-[2-chloro-5-(8-chloro-4-oxo-chromen-2-yl)-4-methoxy-phenoxy]cyclobutanecarboxylate (compound 7c) in Step 4. 3-[5-(8-Chloro-4-oxo-chromen-2-yl)-2-methyl-phenoxy]cyclobutanecarboxylic acid (compound 15b) was obtained as a yellow solid. MS obsd. (ESI⁺) [(M+H)⁺]: 385.

Compound 15b was further purified by supercritical fluid chromatography (SFC) to give cis-3-[5-(8-chloro-4-oxo-chromen-2-yl)-2-methyl-phenoxy]cyclobutanecarboxylic acid (Example 15) and trans-3-[5-(8-chloro-4-oxo-chromen-2-yl)-2-methyl-phenoxy]cyclobutanecarboxylic acid (Example 16). The configuration of Example 15 and Example 16 were determined by NOESY.

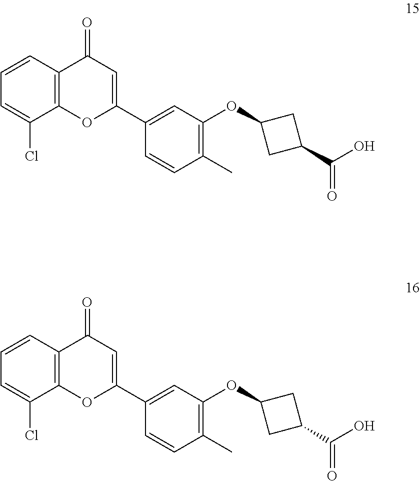 [SMILES: Cc1ccc(-c2cc(=O)c3cccc(Cl)c3o2)cc1O[C@H]1C[C@@H](C(=O)O)C1 ; Cc1ccc(-c2cc(=O)c3cccc(Cl)c3o2)cc1O[C@H]1C[C@H](C(=O)O)C1]

### Example 15

24 mg, 24.1% yield, light yellow solid. ¹H NMR (400 MHz, DMSO-d₆) δ ppm 11.9-12.8 (m, 1H), 7.9-8.1 (m, 2H), 7.6-7.7 (m, 1H), 7.5-7.5 (m, 1H), 7.4-7.5 (m, 1H), 7.3-7.4 (m, 1H), 7.1-7.2 (m, 1H), 4.7-4.9 (m, 1H), 2.7-2.9 (m, 3H), 2.1-2.4 (m, 5H). MS obsd. (ESI⁺) [(M+H)⁺]: 385.

### Example 16

15 mg, 14.8% yield, light yellow solid. ¹H NMR (400 MHz, DMSO-d₆) δ ppm 12.0-12.7 (m, 1H), 7.9-8.1 (m, 2H), 7.6-7.7 (m, 1H), 7.5-7.5 (m, 1H), 7.4-7.5 (m, 1H), 7.3-7.4 (m, 1H), 7.17 (s, 1H), 4.9-5.1 (m, 1H), 3.1-3.2 (m, 1H), 2.7-2.8 (m, 2H), 2.3-2.5 (m, 2H), 2.25 (s, 3H). MS obsd. (ESI⁺) [(M+H)⁺]: 385.

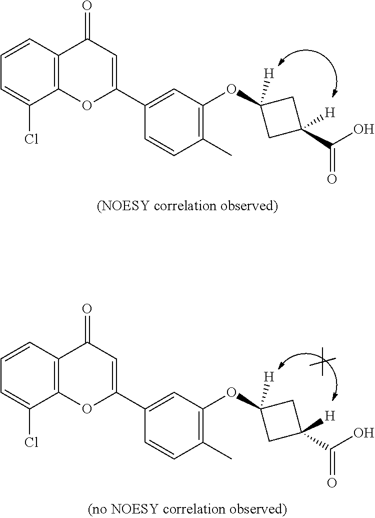 [SMILES: Cc1ccc(-c2cc(=O)c3cccc(Cl)c3o2)cc1O[C@H]1C[C@@H](C(=O)O)C1 ; Cc1ccc(-c2cc(=O)c3cccc(Cl)c3o2)cc1O[C@H]1C[C@H](C(=O)O)C1]

### Example 17

### 3-[5-(8-Chloro-4-oxo-chromen-2-yl)-2-methoxy-phenoxy]cyclobutanecarboxylic acid

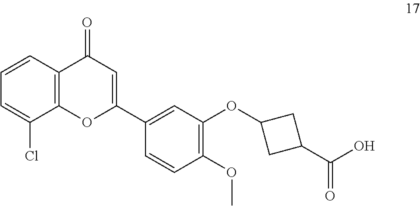 [SMILES: COc1ccc(-c2cc(=O)c3cccc(Cl)c3o2)cc1OC1CC(C(=O)O)C1]

### Step 1. Synthesis of (E)-1-(3-chloro-2-hydroxy-phenyl)-3-(3-hydroxy-4-methoxy-phenyl)prop-2-en-1-one

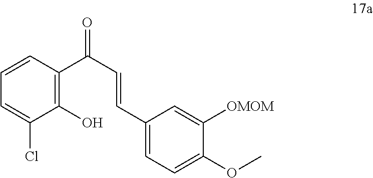

Compound 17a was prepared in analogy to the procedure described for the preparation of example 1 by using 3-hydroxy-4-methoxybenzaldehyde as the starting material instead of 4-bromo-2-methoxy-5-[(4-methoxyphenyl)methoxy]benzaldehyde (Intermediate AA) in Step 1. (E)-1-(3-chloro-2-hydroxy-phenyl)-3-(3-hydroxy-4-methoxy-phenyl)prop-2-en-1-one (4.3 g, 85.9% yield, compound 17a) was obtained as a yellow solid, which was used in the next step directly without further purification. MS obsd. (ESI⁺) [(M+H)⁺]: 305.

### Step 2. Synthesis of 8-chloro-2-(3-hydroxy-4-methoxy-phenyl)chromen-4-one

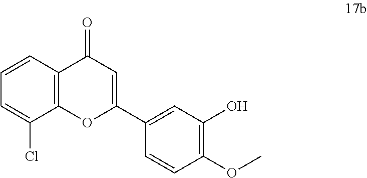 [SMILES: COc1ccc(-c2cc(=O)c3cccc(Cl)c3o2)cc1O]

Compound 17b was prepared in analogy to the procedure described for the preparation of example 1 by using (E)-1-(3-chloro-2-hydroxyphenyl)-3-(3-hydroxy-4-methoxyphenyl)prop-2-en-1-one as the starting material instead of (E)-3-[4-bromo-2-methoxy-5-[(4-methoxyphenyl)methoxy]phenyl]-1-(3-chloro-2-hydroxy-phenyl)prop-2-en-1-one (compound 1a) in Step 2. 8-Chloro-2-(3-hydroxy-4-methoxy-phenyl)chromen-4-one (1.56 g, 78.5% yield, compound 17b) was obtained as a light yellow solid. MS obsd. (ESI⁺) [(M+H)⁺]: 303.

### Step 3. Synthesis of methyl 3-[5-(8-chloro-4-oxo-chromen-2-yl)-2-methoxy-phenoxy]cyclobutanecarboxylate

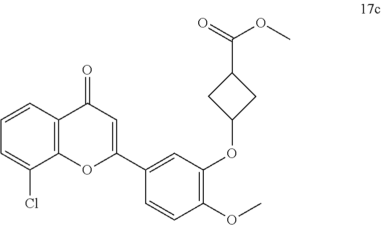 [SMILES: COC(=O)C1CC(Oc2cc(-c3cc(=O)c4cccc(Cl)c4o3)ccc2OC)C1]

Compound 17c was prepared in analogy to the procedure described for the preparation of example 1 by using 8-chloro-2-(3-hydroxy-4-methoxyphenyl)-chromen-4-one (compound 17b) and methyl 3-(p-tolylsulfonyloxy)cyclobutanecarboxylate as the starting material instead of 2-(4-bromo-5-hydroxy-2-methoxy-phenyl)-8-chloro-chromen-4-one (compound 1c) and cis-tert-butyl 3-[2-(p-tolylsulfonyloxy)ethoxy]cyclobutanecarboxylate (Intermediate AP) in Step 4. Methyl 3-[5-(8-chloro-4-oxo-chromen-2-yl)-2-methoxy-phenoxy]cyclobutanecarboxylate (220 mg, 80% yield, compound 17c) was obtained as a yellow solid and the residue was used in the next step directly. MS obsd. (ESI⁺) [(M+H)⁺]: 415.

### Step 4. Synthesis of [5-(8-chloro-4-oxo-chromen-2-yl)-2-methoxy-phenoxy]cyclobutanecarboxylic acid

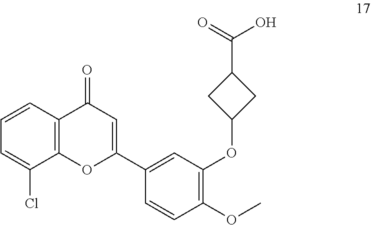 [SMILES: COc1ccc(-c2cc(=O)c3cccc(Cl)c3o2)cc1OC1CC(C(=O)O)C1]

Example 17 was prepared in analogy to the procedure described for the preparation of example 7 by using Methyl 3-[5-(8-chloro-4-oxo-chromen-2-yl)-2-methoxy-phenoxy]cyclobutanecarboxylate (220 mg, compound 17c) as the starting material instead of methyl 3-[2-chloro-5-(8-chloro-4-oxo-chromen-2-yl)-4-methoxy-phenoxy]cyclobutanecarboxylate (compound 7c) in Step 4. 3-[5-(8-Chloro-4-oxo-chromen-2-yl)-2-methoxy-phenoxy]cyclobutanecarboxylic acid (105 mg, 53.3% yield, example 17) was obtained as a yellow foam. ¹H NMR (400 MHz, DMSO-d₆) δ ppm 12.3-12.4 (m, 1H), 8.0-8.0 (m, 2H), 7.7-7.8 (m, 1H), 7.5-7.5 (m, 2H), 7.2-7.2 (m, 1H), 7.1-7.1 (m, 1H), 4.96 (s, 1H), 3.8-3.9 (m, 3H), 2.7-3.1 (m, 3H), 2.4-2.5 (m, 1H), 2.23 (br d, 1H, J=7.5 Hz). MS obsd. (ESI⁺) [(M+H)⁺]: 401.

### Example 18

### 3-[2-[5-(8-Chloro-4-oxo-chromen-2-yl)-2-methoxy-phenoxy]ethoxy]cyclobutanecarboxylic acid

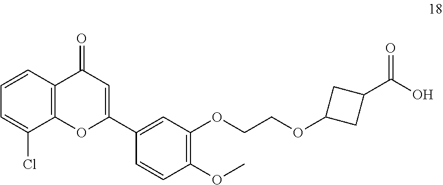 [SMILES: COc1ccc(-c2cc(=O)c3cccc(Cl)c3o2)cc1OCCOC1CC(C(=O)O)C1]

### Step 1. Synthesis of methyl 3-[2-[5-(8-chloro-4-oxo-chromen-2-yl)-2-methoxy-phenoxy]ethoxy]cyclobutanecarboxylate

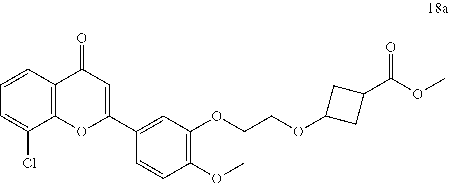 [SMILES: COC(=O)C1CC(OCCOc2cc(-c3cc(=O)c4cccc(Cl)c4o3)ccc2OC)C1]

Compound 18a was prepared in analogy to the procedure described for the preparation of example 1 by using 8-chloro-2-(3-hydroxy-4-methoxyphenyl)-chromen-4-one and methyl 3-(2-(tosyloxy)ethoxy)cyclobutane-1-carboxylate as the starting material (Intermediate AO) instead of 2-(4-bromo-5-hydroxy-2-methoxy-phenyl)-8-chloro-chromen-4-one (compound 1c) and cis-tert-butyl 3-[2-(p-tolylsulfonyloxy)ethoxy]cyclobutanecarboxylate (Intermediate AP) in Step 4. Methyl 3-[2-[5-(8-chloro-4-oxo-chromen-2-yl)-2-methoxy-phenoxy]ethoxy]cyclobutanecarboxylate (250 mg, 82.5% yield, compound 18a) was obtained as a yellow oil and the residue was used in the next step directly. MS obsd. (ESI⁺) [(M+H)⁺]: 459.

### Step 2. Synthesis of 3-[2-[5-(8-chloro-4-oxo-chromen-2-yl)-2-methoxy-phenoxy]ethoxy]cyclobutanecarboxylic acid

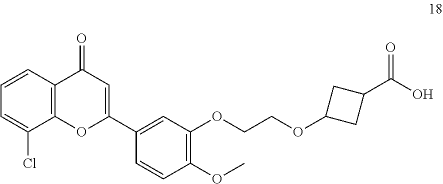 [SMILES: COc1ccc(-c2cc(=O)c3cccc(Cl)c3o2)cc1OCCOC1CC(C(=O)O)C1]

Example 18 was prepared in analogy to the procedure described for the preparation of example 7 by using methyl 3-[2-[5-(8-chloro-4-oxo-chromen-2-yl)-2-methoxy-phenoxy]ethoxy]cyclobutanecarboxylate (250 mg, compound 18a) as the starting material instead of methyl 3-[2-chloro-5-(8-chloro-4-oxo-chromen-2-yl)-4-methoxy-phenoxy]cyclobutanecarboxylate (compound 7c) in Step 4. 3-[2-[5-(8-chloro-4-oxo-chromen-2-yl)-2-methoxy-phenoxy]ethoxy]cyclobutanecarboxylic acid (100 mg, 50% yield, example 18) was obtained as a light yellow powder. ¹H NMR (400 MHz, DMSO-d₆) δ ppm 12.1-12.2 (m, 1H), 7.99 (d, J=8.1 Hz, 2H), 7.7-7.8 (m, 1H), 7.6-7.7 (m, 1H), 7.4-7.5 (m, 1H), 7.17 (s, 2H), 4.1-4.3 (m, 2.5H), 3.97 (t, 0.5H, J=6.9 Hz), 3.88 (s, 3H), 3.9-4.3 (m, 3H), 2.5-3.0 (m, 1H), 2.4-2.5 (m, 2H), 2.1-2.2 (m, 1H), 2.0-2.1 (m, 1H). MS obsd. (ESI⁺) [(M+H)⁺]: 445.

### Example 19

### 3-[5-(8-Chloro-4-oxo-chromen-2-yl)-2-methoxy-phenoxy]propanoic acid

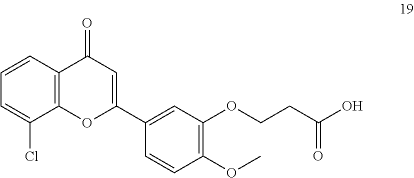 [SMILES: COc1ccc(-c2cc(=O)c3cccc(Cl)c3o2)cc1OCCC(=O)O]

Example 19 was prepared in analogy to the procedure described for the preparation of example 2 by using 8-chloro-2-(3-hydroxy-4-methoxyphenyl)-chromen-4-one as the starting material instead of 2-(4-bromo-5-hydroxy-2-methoxy-phenyl)-8-chloro-chromen-4-one (compound 1c). 3-[5-(8-Chloro-4-oxo-chromen-2-yl)-2-methoxy-phenoxy]propanoic acid (33.8 mg, 13% yield, example 19) was obtained as an off-white powder. ¹H NMR (400 MHz, DMSO-d₆) δ ppm 12.41 (br s, 1H), 8.00 (dd, J=1.2, 7.9 Hz, 2H), 7.7-7.8 (m, 1H), 7.6-7.7 (m, 1H), 7.4-7.5 (m, 1H), 7.19 (s, 2H), 4.31 (t, J=6.1 Hz, 2H), 3.86 (s, 3H), 2.75 (t, J=6.0 Hz, 2H). MS obsd. (ESI⁺) [(M+H)⁺]: 375.

### Example 20 and Example 21

### Cis-3-[5-(8-chloro-4-oxo-chromen-2-yl)-2-(trifluoromethyl)phenoxy]cyclobutanecarboxylic acid and trans-3-[5-(8-chloro-4-oxo-chromen-2-yl)-2-(trifluoromethyl)phenoxy]cyclobutanecarboxylic acid

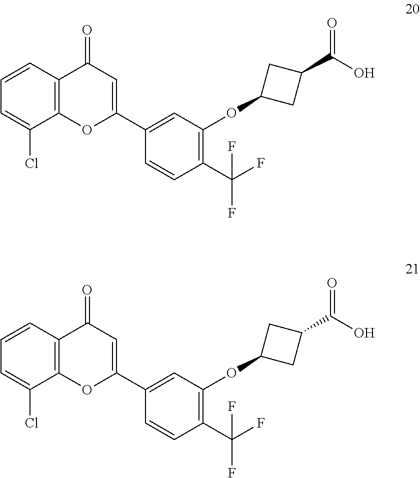 [SMILES: O=c1cc(-c2ccc(C(F)(F)F)c(O[C@H]3C[C@@H](C(=O)O)C3)c2)oc2c(Cl)cccc12 ; O=c1cc(-c2ccc(C(F)(F)F)c(O[C@H]3C[C@H](C(=O)O)C3)c2)oc2c(Cl)cccc12]

### Step 1. Synthesis of (E)-1-(3-chloro-2-hydroxy-phenyl)-3-[3-methoxy-4-(trifluoromethyl)phenyl]prop-2-en-1-one

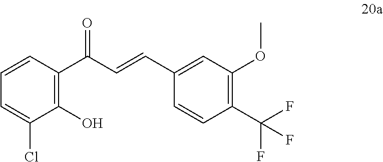 [SMILES: COc1cc(/C=C/C(=O)c2cccc(Cl)c2O)ccc1C(F)(F)F]

Compound 20a was prepared in analogy to the procedure described for the preparation of example 1 by using 3-methoxy-4-(trifluoromethyl)benzaldehyde as the starting material instead of 4-bromo-2-methoxy-5-[(4-methoxyphenyl)methoxy]benzaldehyde (Intermediate AA) in Step 1. (E)-1-(3-chloro-2-hydroxy-phenyl)-3-[3-methoxy-4-(trifluoromethyl)phenyl]prop-2-en-1-one (1.5 g, 85.8% yield, compound 20a) was obtained as yellow solid, which was used in the next step directly without further purification. (ESI⁺) [(M+H)⁺]: 357.

### Step 2. Synthesis of 8-chloro-2-(3-methoxy-4-(trifluoromethyl)phenyl)-chromen-4-one

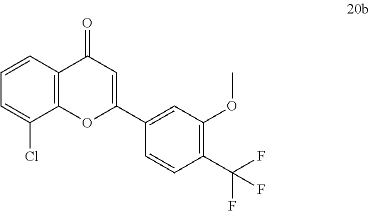 [SMILES: COc1cc(-c2cc(=O)c3cccc(Cl)c3o2)ccc1C(F)(F)F]

Compound 20b was prepared in analogy to the procedure described for the preparation of example 1 by using (E)-1-(3-chloro-2-hydroxy-phenyl)-3-[3-methoxy-4-(trifluoromethyl)phenyl]prop-2-en-1-one (1.5 g, compound 20a) as the starting material instead of (E)-3-[4-bromo-2-methoxy-5-[(4-methoxyphenyl)methoxy]phenyl]-1-(3-chloro-2-hydroxy-phenyl)prop-2-en-1-one (compound 1a) in Step 2. 8-chloro-2-(3-methoxy-4-(trifluoromethyl)phenyl)-chromen-4-one (900 mg, 90.5% yield, compound 20b) was obtained as light yellow solid. MS obsd. (ESI⁺) [(M+H)⁺]: 355.

### Step 3. Synthesis of 8-chloro-2-(3-hydroxy-4-(trifluoromethyl)phenyl)-chromen-4-one

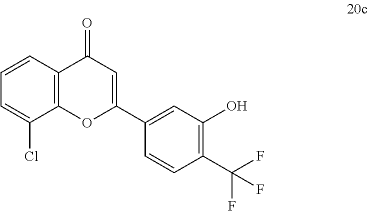 [SMILES: O=c1cc(-c2ccc(C(F)(F)F)c(O)c2)oc2c(Cl)cccc12]

The solution of 8-chloro-2-(3-methoxy-4-(trifluoromethyl)phenyl)-chromen-4-one (300 mg) in DCM (20 mL) was stirred at 25° C. for 14 hrs. EtOAc and water were poured into the reaction mixture. The organic layer was washed with brine, dried by Na₂SO₄, concentrated to give 8-chloro-2-(3-hydroxy-4-(trifluoromethyl)phenyl)-chromen-4-one (270 mg, 93.7% yield, compound 20c) as a yellow solid, which was used in the next step directly without further purification. MS obsd. (ESI⁺) [(M+H)⁺]: 341.

### Step 4. Synthesis of methyl 3-(5-(8-chloro-4-oxo-chromen-2-yl)-2-(trifluoromethyl)phenoxy)cyclobutane-1-carboxylate

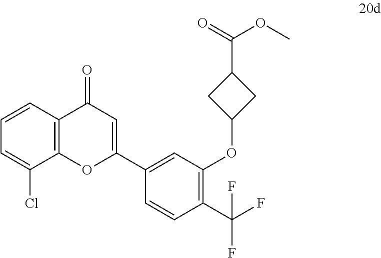 [SMILES: COC(=O)C1CC(Oc2cc(-c3cc(=O)c4cccc(Cl)c4o3)ccc2C(F)(F)F)C1]

Compound 20d was prepared in analogy to the procedure described for the preparation of example 1 by using 8-chloro-2-(3-hydroxy-4-(trifluoromethyl)phenyl)-chromen-4-one (compound 20c) and methyl 3-chlorocyclobutane-1-carboxylate as the starting material instead of 2-(4-bromo-5-hydroxy-2-methoxy-phenyl)-8-chloro-chromen-4-one (compound 1c) and cis-tert-butyl 3-[2-(p-tolylsulfonyloxy)ethoxy]cyclobutanecarboxylate (Intermediate AP) in Step 4. Methyl 3-(5-(8-chloro-4-oxo-chromen-2-yl)-2-(trifluoromethyl)phenoxy)cyclobutane-1-carboxylate (100 mg, 75.2% yield, compound 20d) was obtained as a yellow oil and the residue was used in the next step directly. MS obsd. (ESI⁺) [(M+H)⁺]: 453.

### Step 5. Synthesis of 3-[5-(8-chloro-4-oxo-chromen-2-yl)-2-(trifluoromethyl)phenoxy]cyclobutanecarboxylic acid

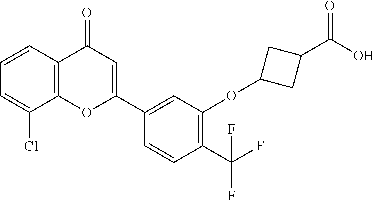 [SMILES: O=C(O)C1CC(Oc2cc(-c3cc(=O)c4cccc(Cl)c4o3)ccc2C(F)(F)F)C1]

### 20e

Compound 20e was prepared in analogy to the procedure described for the preparation of example 7 by using methyl 3-(5-(8-chloro-4-oxo-chromen-2-yl)-2-(trifluoromethyl)phenoxy)cyclobutane-1-carboxylate (compound 20d) as the starting material instead of methyl 3-[2-chloro-5-(8-chloro-4-oxo-chromen-2-yl)-4-methoxy-phenoxy]cyclobutanecarboxylate (compound 7c) in Step 4. 3-[5-(8-chloro-4-oxo-chromen-2-yl)-2-(trifluoromethyl)phenoxy]cyclobutanecarboxylic acid (compound 20e) was obtained as a yellow solid. MS obsd. (ESI⁺) [(M+H)⁺]: 439.

Separation of 20e by Pre-HPLC give 3-[5-(8-chloro-4-oxo-chromen-2-yl)-2-(trifluoromethyl)phenoxy]cyclobutanecarboxylic acid with cis- and trans-configuration, one of which is characterized as cis-configuration (Example 20, 3.2 mg, 3%) and the other is characterized as trans-configuration (Example 21, 3.0 mg, 2.8%). (Separation condition: H₂O (0.1% FA) and ACN (flow rate: 25 ml/min) on waters sun fire C18 Column. The configuration of Example 20 and Example 21 were determined by NOESY

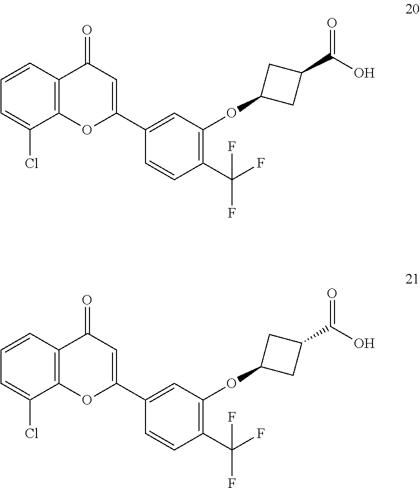 [SMILES: O=c1cc(-c2ccc(C(F)(F)F)c(O[C@H]3C[C@@H](C(=O)O)C3)c2)oc2c(Cl)cccc12 ; O=c1cc(-c2ccc(C(F)(F)F)c(O[C@H]3C[C@H](C(=O)O)C3)c2)oc2c(Cl)cccc12]

### Example 20

¹H NMR (DMSO-d₆) δ ppm 12.12-12.53 (m, 1H), 8.04 (ddd, J=9.48, 7.95, 1.53 Hz, 2H), 7.81-7.89 (m, 2H), 7.71 (s, 1H), 7.37 (s, 1H), 7.53 (t, J=7.89 Hz, 1H), 6.51 (s, 1H), 4.92-5.08 (m, 1H), 2.75-2.90 (m, 3H), 2.17-2.30 (m, 2H). MS obsd. (ESI⁺) [(M+H)⁺]: 439.

### Example 21

¹H NMR (400 MHz, DMSO-d₆) δ ppm 12.23-12.61 (m, 1H), 8.04 (ddd, J=10.21, 7.95, 1.53 Hz, 2H), 7.86 (s, 2H), 7.69 (s, 1H), 7.53 (t, J=7.89 Hz, 1H), 7.38 (s, 1H), 5.19 (t, J=6.66 Hz, 1H), 3.08-3.18 (m, 1H), 2.80 (ddd, J=13.51, 7.09, 3.73 Hz, 2H), 2.34-2.45 (m, 3H). MS obsd. (ESI⁺) [(M+H)⁺]: 439.

### Example 22

### 2-[5-(8-Chloro-4-oxo-chromen-2-yl)-2-(trifluoromethyl)phenoxy]acetic acid

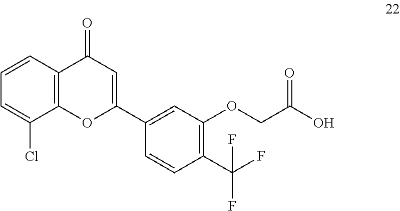 [SMILES: O=C(O)COc1cc(-c2cc(=O)c3cccc(Cl)c3o2)ccc1C(F)(F)F]

### Step 1. Synthesis of ethyl 2-(5-(8-chloro-4-oxo-chromen-2-yl)-2-(trifluoromethyl)phenoxy)acetate

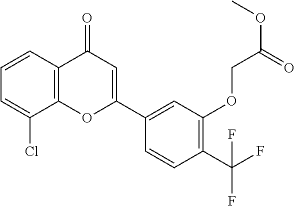 [SMILES: COC(=O)COc1cc(-c2cc(=O)c3cccc(Cl)c3o2)ccc1C(F)(F)F]

Compound 22a was prepared in analogy to the procedure described for the preparation of example 1 by using 8-chloro-2-(3-hydroxy-4-(trifluoromethyl)phenyl)-chromen-4-one (compound 20c) and ethyl 2-bromoacetate as the starting material instead of 2-(4-bromo-5-hydroxy-2-methoxy-phenyl)-8-chloro-chromen-4-one (compound 1c) and cis-tert-butyl 3-[2-(p-tolylsulfonyloxy)ethoxy]cyclobutanecarboxylate (Intermediate AP) in Step 4. Ethyl 2-(5-(8-chloro-4-oxo-chromen-2-yl)-2-(trifluoromethyl)phenoxy)acetate (100 mg, 79.8% yield, compound 22a) was obtained as a yellow solid and the residue was used in the next step directly. MS obsd. (ESI⁺) [(M+H)⁺].

### Step 2. Synthesis of 2-[5-(8-chloro-4-oxo-chromen-2-yl)-2-(trifluoromethyl)phenoxy]acetic acid

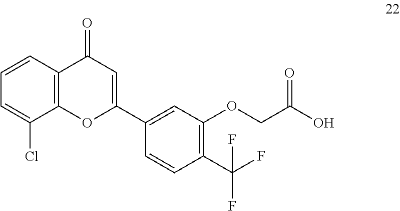 [SMILES: O=C(O)COc1cc(-c2cc(=O)c3cccc(Cl)c3o2)ccc1C(F)(F)F]

Example 22 was prepared in analogy to the procedure described for the preparation of example 7 by using ethyl 2-(5-(8-chloro-4-oxo-chromen-2-yl)-2-(trifluoromethyl)phenoxy)acetate (compound 22a) as the starting material instead of methyl 3-[2-chloro-5-(8-chloro-4-oxo-chromen-2-yl)-4-methoxy-phenoxy]cyclobutanecarboxylate (compound 7c) in Step 4. 2-[5-(8-chloro-4-oxo-chromen-2-yl)-2-(trifluoromethyl)phenoxy]acetic acid (13 mg, 13.2% yield, Example 22) was obtained as a white foam. ¹H NMR (400 MHz, DMSO-d₆) δ ppm 8.03 (ddd, 2H, J=1.5, 7.9, 10.2 Hz), 7.8-7.9 (m, 3H), 7.53 (t, 1H, J=7.9 Hz), 7.41 (s, 1H), 5.05 (s, 2H). MS obsd. (ESI⁺) [(M+H)⁺]: 399.

### Example 23

### Cis-3-[5-(8-chloro-4-oxo-chromen-2-yl)-2,3-dimethoxy-phenoxy]cyclobutanecarboxylic acid

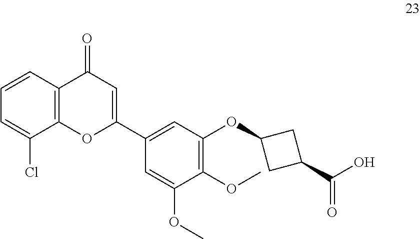 [SMILES: COc1cc(-c2cc(=O)c3cccc(Cl)c3o2)cc(O[C@H]2C[C@@H](C(=O)O)C2)c1OC]

### Step 1. (E)-1-(3-chloro-2-hydroxyphenyl)-3-(3-hydroxy-4,5-dimethoxyphenyl)prop-2-en-1-one

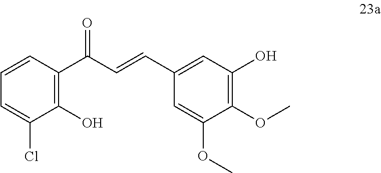 [SMILES: COc1cc(/C=C/C(=O)c2cccc(Cl)c2O)cc(O)c1OC]

Compound 23a was prepared in analogy to the procedure described for the preparation of example 1 by using 3-hydroxy-4,5-dimethoxybenzaldehyde as the starting material instead of 4-bromo-2-methoxy-5-[(4-methoxyphenyl)methoxy]benzaldehyde (Intermediate AA) in Step 1. (E)-1-(3-chloro-2-hydroxyphenyl)-3-(3-hydroxy-4,5-dimethoxyphenyl)prop-2-en-1-one (3.5 g, 89.2% yield, compound 23a) was obtained as a yellow solid, which was used in the next step directly without further purification. MS obsd. (ESI⁺) [(M+H)⁺]: 335.

### Step 2. Synthesis of 8-chloro-2-(3-hydroxy-4,5-dimethoxyphenyl)-chromen-4-one

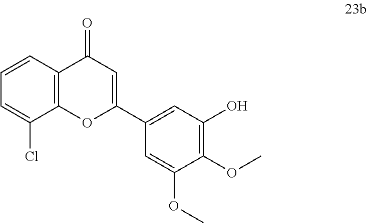 [SMILES: COc1cc(-c2cc(=O)c3cccc(Cl)c3o2)cc(O)c1OC]

Compound 23b was prepared in analogy to the procedure described for the preparation of example 1 by using (E)-1-(3-chloro-2-hydroxyphenyl)-3-(3-hydroxy-4,5-dimethoxyphenyl)prop-2-en-1-one (compound 23a) as the starting material instead of (E)-3-[4-bromo-2-methoxy-5-[(4-methoxyphenyl)methoxy]phenyl]-1-(3-chloro-2-hydroxy-phenyl)prop-2-en-1-one (compound 1a) in Step 2. 8-chloro-2-(3-hydroxy-4,5-dimethoxyphenyl)-chromen-4-one (1.5 g, 75.5% yield, compound 23b) was obtained as yellow solid. MS obsd. (ESI⁺) [(M+H)⁺]: 333.

### Step 3. Synthesis of methyl 3-[5-(8-chloro-4-oxo-chromen-2-yl)-2,3-dimethoxy-phenoxy]cyclobutanecarboxylate

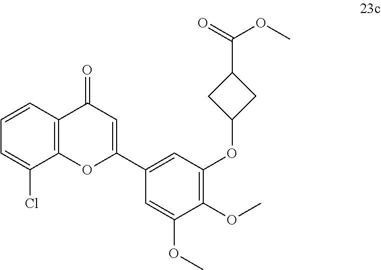 [SMILES: COC(=O)C1CC(Oc2cc(-c3cc(=O)c4cccc(Cl)c4o3)cc(OC)c2OC)C1]

Compound 23c was prepared in analogy to the procedure described for the preparation of example 1 by using 8-chloro-2-(3-hydroxy-4,5-dimethoxyphenyl)-chromen-4-one (1.5 g compound 23b) and methyl 3-(p-tolylsulfonyloxy)cyclobutanecarboxylate as the starting material instead of 2-(4-bromo-5-hydroxy-2-methoxy-phenyl)-8-chloro-chromen-4-one (compound 1c) and cis-tert-butyl 3-[2-(p-tolylsulfonyloxy)ethoxy]cyclobutanecarboxylate in Step 4. Methyl 3-[5-(8-chloro-4-oxo-chromen-2-yl)-2,3-dimethoxy-phenoxy]cyclobutanecarboxylate (350 mg, 87.3% yield, compound 23c) was obtained as a yellow oil.

Separation of 23c by Pre-HPLC give methyl 3-[5-(8-chloro-4-oxo-chromen-2-yl)-2,3-dimethoxy-phenoxy]cyclobutanecarboxylate with cis- and trans-configuration, one of which is characterized as cis-configuration (Example 23c-1) and the other is characterized as trans-configuration (Example 23c-2). MS obsd. (ESI⁺) [(M+H)⁺]: 445.

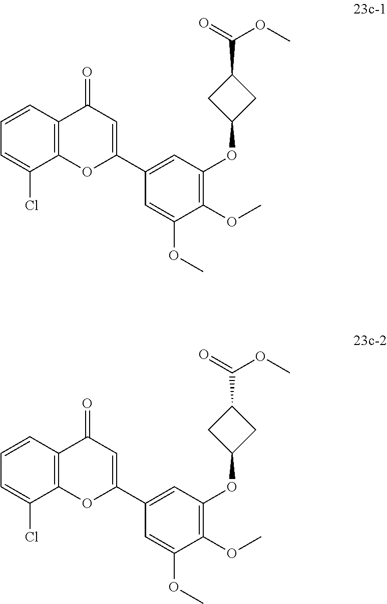 [SMILES: COc1cc(-c2cc(=O)c3cccc(Cl)c3o2)cc(O[C@H]2C[C@@H](C(=O)OC)C2)c1OC ; COc1cc(-c2cc(=O)c3cccc(Cl)c3o2)cc(O[C@H]2C[C@H](C(=O)OC)C2)c1OC]

### Step 4. Synthesis of cis-3-[5-(8-chloro-4-oxo-chromen-2-yl)-2,3-dimethoxy-phenoxy]cyclobutanecarboxylic acid

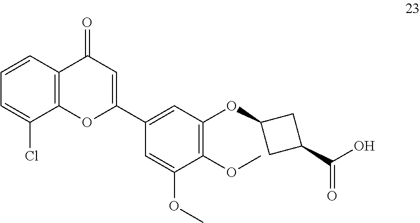 [SMILES: COc1cc(-c2cc(=O)c3cccc(Cl)c3o2)cc(O[C@H]2C[C@@H](C(=O)O)C2)c1OC]

Compound 23 was prepared in analogy to the procedure described for the preparation of example 7 by using cis-methyl-3-(5-(8-chloro-4-oxo-chromen-2-yl)-2,3-dimethoxyphenoxy)cyclobutane-1-carboxylate (compound 23c-1) as the starting material instead of methyl 3-[2-chloro-5-(8-chloro-4-oxo-chromen-2-yl)-4-methoxy-phenoxy]cyclobutanecarboxylate (compound 7c) in Step 4. Cis-3-[5-(8-chloro-4-oxo-chromen-2-yl)-2,3-dimethoxy-phenoxy]cyclobutanecarboxylic acid (40.5 mg, 79.5% yield, example 23) was obtained as an off-white solid ¹H NMR (400 MHz, DMSO-d₆) δ ppm 12.30 (br s, 1H), 8.01 (br t, J=7.2 Hz, 2H), 7.50 (br t, J=7.8 Hz, 1H), 7.42 (s, 1H), 7.27 (s, 2H), 4.79 (br d, J=6.5 Hz, 1H), 3.90 (s, 3H), 3.7-3.8 (m, 3H), 2.7-2.9 (m, 3H), 2.2-2.3 (m, 2H). MS obsd. (ESI⁺) [(M+H)⁺]: 431.

### Example 24

### Trans-3-[2-[5-(8-chloro-4-oxo-chromen-2-yl)-2,3-dimethoxy-phenoxy]ethoxy]cyclobutanecarboxylic acid

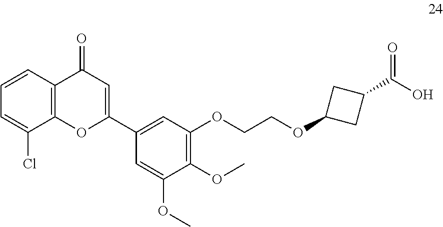 [SMILES: COc1cc(-c2cc(=O)c3cccc(Cl)c3o2)cc(OCCO[C@H]2C[C@H](C(=O)O)C2)c1OC]

### Step 1. Synthesis of methyl 3-[2-[5-(8-chloro-4-oxo-chromen-2-yl)-2,3-dimethoxy-phenoxy]ethoxy]cyclobutanecarboxylate

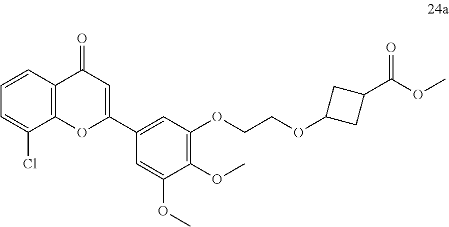 [SMILES: COC(=O)C1CC(OCCOc2cc(-c3cc(=O)c4cccc(Cl)c4o3)cc(OC)c2OC)C1]

Compound 24a was prepared in analogy to the procedure described for the preparation of example 1 by using 8-chloro-2-(3-hydroxy-4,5-dimethoxyphenyl)-chromen-4-one (compound 23b) and methyl 3-(2-(tosyloxy)ethoxy)cyclobutane-1-carboxylate (Intermediate AO) as the starting material instead of 2-(4-bromo-5-hydroxy-2-methoxy-phenyl)-8-chloro-chromen-4-one (compound 1c) and cis-tert-butyl 3-[2-(p-tolylsulfonyloxy)ethoxy]cyclobutanecarboxylate (Intermediate AP) in Step 4. Methyl 3-[2-[5-(8-chloro-4-oxo-chromen-2-yl)-2,3-dimethoxy-phenoxy]ethoxy]cyclobutanecarboxylate (compound 24a) was obtained as a yellow solid. MS obsd. (ESI⁺) [(M+H)⁺]: 489.

Separation of compound 24a give methyl-3-(2-(5-(8-chloro-4-oxo-chromen-2-yl)-2,3-dimethoxyphenoxy)ethoxy)cyclobutane-1-carboxylate as a yellow solid with cis- and trans-configuration, one of which is characterized as cis-configuration (compound 24a-1) (45 mg, 22.5%) and the other is characterized as trans-configuration (compound 24a-2) (36 mg, 18%). MS obsd. (ESI⁺) [(M+H)⁺]: 489. Separation condition: H₂O (0.1% FA) and ACN on waters sun fire C18 column (flow rate: 25 mL/minute).

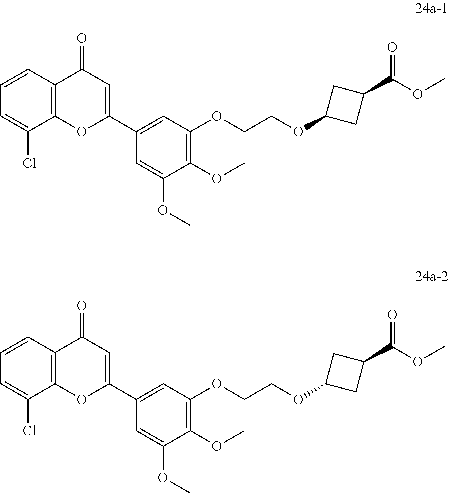 [SMILES: COc1cc(-c2cc(=O)c3cccc(Cl)c3o2)cc(OCCO[C@H]2C[C@@H](C(=O)OC)C2)c1OC ; COc1cc(-c2cc(=O)c3cccc(Cl)c3o2)cc(OCCO[C@H]2C[C@H](C(=O)OC)C2)c1OC]

### Step 2. Synthesis of trans-3-[2-[5-(8-chloro-4-oxo-chromen-2-yl)-2,3-dimethoxy-phenoxy]ethoxy]cyclobutanecarboxylic acid

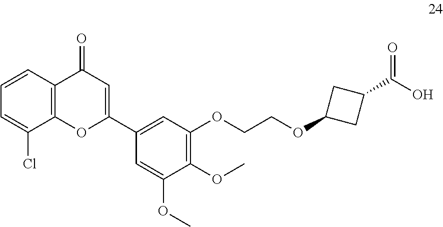 [SMILES: COc1cc(-c2cc(=O)c3cccc(Cl)c3o2)cc(OCCO[C@H]2C[C@H](C(=O)O)C2)c1OC]

Example 24 was prepared in analogy to the procedure described for the preparation of example 7 by using trans-methyl-3-(2-(5-(8-chloro-4-oxo-chromen-2-yl)-2,3-dimethoxyphenoxy)ethoxy)cyclobutane-1-carboxylate (36 mg, compound 24a-2) as the starting material instead of methyl 3-[2-chloro-5-(8-chloro-4-oxo-chromen-2-yl)-4-methoxy-phenoxy]cyclobutanecarboxylate (compound 7c) in Step 4. Trans-3-[2-[5-(8-chloro-4-oxo-chromen-2-yl)-2,3-dimethoxy-phenoxy]ethoxy]cyclobutanecarboxylic acid (1.8 mg, 4.5% yield, Example 24) was obtained as an off-white foam. ¹H NMR (400 MHz, DMSO-d₆) δ ppm 12.17 (br s, 1H), 8.0-8.1 (m, 2H), 7.51 (s, 1H), 7.43 (d, J=2.3 Hz, 2H), 7.28 (s, 1H), 4.2-4.3 (m, 3H), 3.91 (s, 3H), 3.80 (s, 3H), 3.6-3.7 (m, 2H), 2.8-3.0 (m, 1H), 2.4-2.4 (m, 2H), 2.1-2.2 (m, 2H). MS obsd. (ESI⁺) [(M+H)⁺]: 475.

### Example 25

### 2-[5-(8-Chloro-4-oxo-chromen-2-yl)-2,3-dimethoxy-phenoxy]acetic acid

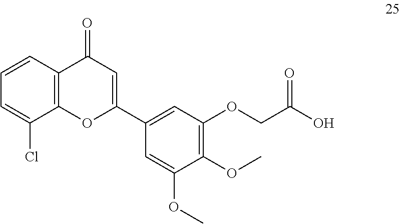 [SMILES: COc1cc(-c2cc(=O)c3cccc(Cl)c3o2)cc(OCC(=O)O)c1OC]

### Step 1. Synthesis of ethyl 2-(5-(8-chloro-4-oxo-chromen-2-yl)-2,3-dimethoxyphenoxy)acetate

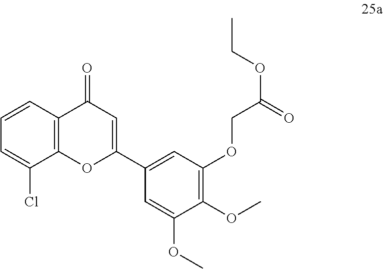 [SMILES: CCOC(=O)COc1cc(-c2cc(=O)c3cccc(Cl)c3o2)cc(OC)c1OC]

Compound 25a was prepared in analogy to the procedure described for the preparation of example 1 by using 8-chloro-2-(3-hydroxy-4,5-dimethoxyphenyl)-chromen-4-one (compound 23b) and ethyl 2-bromoacetate as the starting material instead of 2-(4-bromo-5-hydroxy-2-methoxy-phenyl)-8-chloro-chromen-4-one (compound 1c) and cis-tert-butyl 3-[2-(p-tolylsulfonyloxy)ethoxy]cyclobutanecarboxylate (Intermediate AP) in Step 4. Ethyl 2-(5-(8-chloro-4-oxo-chromen-2-yl)-2,3-dimethoxyphenoxy)acetate (240 mg, 95.3% yield, compound 25a) was obtained as a yellow solid, which was used in the next step directly. MS obsd. (ESI⁺) [(M+H)⁺]: 405.

### Step 2. Synthesis of 2-[5-(8-chloro-4-oxo-chromen-2-yl)-2,3-dimethoxy-phenoxy]acetic acid

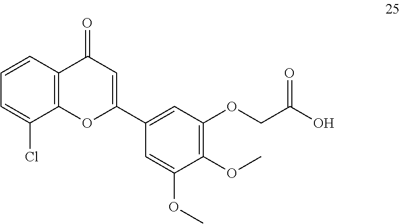 [SMILES: COc1cc(-c2cc(=O)c3cccc(Cl)c3o2)cc(OCC(=O)O)c1OC]

Example 25 was prepared in analogy to the procedure described for the preparation of example 7 by using ethyl 2-(5-(8-chloro-4-oxo-chromen-2-yl)-2,3-dimethoxyphenoxy)acetate (compound 25a) as the starting material instead of methyl 3-[2-chloro-5-(8-chloro-4-oxo-chromen-2-yl)-4-methoxy-phenoxy]cyclobutanecarboxylate (compound 7c) in Step 4. 2-[5-(8-Chloro-4-oxo-chromen-2-yl)-2,3-dimethoxy-phenoxy]acetic acid (83.2 mg, 56% yield, example 25) was obtained as a yellow foam. ¹H NMR (400 MHz, DMSO-d₆) δ ppm 13.11 (br s, 1H), 8.00 (ddd, J=1.5, 6.7, 8.0 Hz, 2H), 7.4-7.5 (m, 2H), 7.36 (d, J=2.0 Hz, 1H), 7.28 (s, 1H), 4.86 (s, 2H), 3.91 (s, 3H), 3.82 (s, 3H). MS obsd. (ESI⁺) [(M+H)⁺]: 391.

### Example 26

### Trans-3-[4-bromo-5-(8-chloro-4-oxo-chromen-2-yl)-2-methoxy-phenoxy]cyclobutanecarboxylic acid

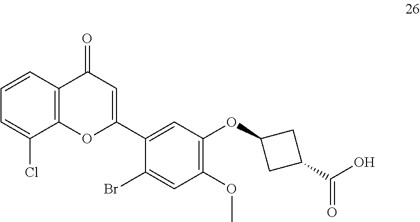 [SMILES: COc1cc(Br)c(-c2cc(=O)c3cccc(Cl)c3o2)cc1O[C@H]1C[C@H](C(=O)O)C1]

### Step 1. Synthesis of (E)-3-(2-bromo-5-hydroxy-4-methoxy-phenyl)-1-(3-chloro-2-hydroxy-phenyl)prop-2-en-1-one

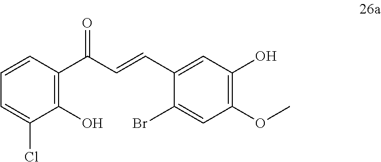 [SMILES: COc1cc(Br)c(/C=C/C(=O)c2cccc(Cl)c2O)cc1O]

Compound 26a was prepared in analogy to the procedure described for the preparation of example 1 by using 2-bromo-5-hydroxy-4-methoxybenzaldehyde as the starting material instead of 4-bromo-2-methoxy-5-[(4-methoxyphenyl)methoxy]benzaldehyde (Intermediate AA) in Step 1. (E)-3-(2-Bromo-5-hydroxy-4-methoxy-phenyl)-1-(3-chloro-2-hydroxy-phenyl)prop-2-en-1-one (4.3 g, 95.6% yield, compound 26a) was obtained as a yellow solid, which was used in the next step without further purification. MS obsd. (ESI⁺) [(M+H)⁺]: 383.

### Step 2. Synthesis of 2-(2-bromo-5-hydroxy-4-methoxy-phenyl)-8-chloro-chromen-4-one

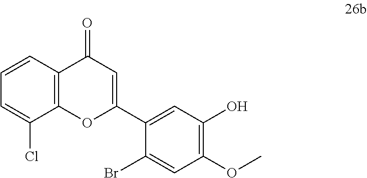 [SMILES: COc1cc(Br)c(-c2cc(=O)c3cccc(Cl)c3o2)cc1O]

Compound 26b was prepared in analogy to the procedure described for the preparation of example 1 by (E)-3-(2-bromo-5-hydroxy-4-methoxyphenyl)-1-(3-chloro-2-hydroxyphenyl)prop-2-en-1-one (compound 26a) as the starting material instead of (E)-3-[4-bromo-2-methoxy-5-[(4-methoxyphenyl)methoxy]phenyl]-1-(3-chloro-2-hydroxy-phenyl)prop-2-en-1-one (compound 1a) in Step 2. 2-(2-Bromo-5-hydroxy-4-methoxy-phenyl)-8-chloro-chromen-4-one (1.7 g, 85.4% yield, compound 26b) was obtained as a yellow solid. (MS obsd. (ESI⁺) [(M+H)⁺]: 382.

### Step 3. Synthesis of methyl 3-[4-bromo-5-(8-chloro-4-oxo-chromen-2-yl)-2-methoxy-phenoxy]cyclobutanecarboxylate

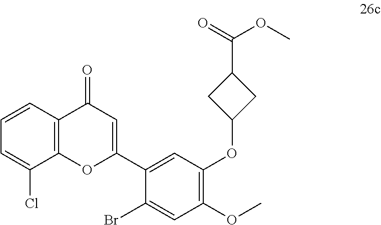 [SMILES: COC(=O)C1CC(Oc2cc(-c3cc(=O)c4cccc(Cl)c4o3)c(Br)cc2OC)C1]

Compound 26c was prepared in analogy to the procedure described for the preparation of example 1 by using 2-(2-bromo-5-hydroxy-4-methoxy-phenyl)-8-chloro-chromen-4-one (1.7 g, compound 26b) and methyl 3-chlorocyclobutane-1-carboxylate as the starting material instead of 2-(4-bromo-5-hydroxy-2-methoxy-phenyl)-8-chloro-chromen-4-one (compound 1c) and cis-tert-butyl 3-[2-(p-tolylsulfonyloxy)ethoxy]cyclobutanecarboxylate (Intermediate AP) in Step 4. Methyl 3-(4-bromo-5-(8-chloro-4-oxo-chromen-2-yl)-2-methoxyphenoxy)cyclobutane-1-carboxylate (compound 26c) was obtained as a yellow solid.

Separation of compound 26c with Semi-preparative HPLC give methyl 3-[4-bromo-5-(8-chloro-4-oxo-chromen-2-yl)-2-methoxy-phenoxy]cyclobutanecarboxylate with cis- and trans-configuration, one of which is characterized as cis-configuration (compound 26c-1,113 mg, 35.8%) and the other is characterized as trans-configuration (compound 26c-2, 55 mg, 17.4%). MS obsd. (ESI⁺) [(M+H)⁺]: 493. (Separation condition: H₂O (10 mM ammonium hydrogen carbonate) and ACN on YMC-Triart Prep C18, 150×40.0 mm, 7 μM (flow rate: 60 mL/min) Step 4. Synthesis of trans-3-[4-bromo-5-(8-chloro-4-oxo-chromen-2-yl)-2-methoxy-phenoxy]cyclobutanecarboxylic acid

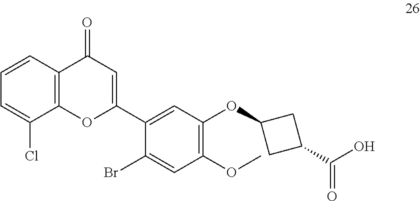 [SMILES: COc1cc(Br)c(-c2cc(=O)c3cccc(Cl)c3o2)cc1O[C@H]1C[C@H](C(=O)O)C1]

Example 26 was prepared in analogy to the procedure described for the preparation of example 7 by using trans-methyl-3-(4-bromo-5-(8-chloro-4-oxo-chromen-2-yl)-2-methoxyphenoxy)cyclobutane-1-carboxylate (45 mg, compound 26c-2) as the starting material instead of methyl 3-[2-chloro-5-(8-chloro-4-oxo-chromen-2-yl)-4-methoxy-phenoxy]cyclobutanecarboxylate (compound 7c) in Step 4. Trans-3-[4-bromo-5-(8-chloro-4-oxo-chromen-2-yl)-2-methoxy-phenoxy]cyclobutanecarboxylic acid (39.7 mg, 90.8% yield, example 26) was obtained as an off-white solid. ¹H NMR (400 MHz, DMSO-d₆) δ ppm 12.25 (br s, 1H), 8.0-8.1 (m, 2H), 7.5-7.6 (m, 1H), 7.38 (s, 1H), 7.19 (s, 1H), 6.72 (s, 1H), 4.8-5.0 (m, 1H), 3.89 (s, 3H), 3.0-3.1 (m, 1H), 2.6-2.7 (m, 2H), 2.3-2.4 (m, 2H). MS obsd. (ESI⁺) [(M+H)⁺]: 480.

### Example 27

### Trans-3-[2-[4-Bromo-5-(8-chloro-4-oxo-chromen-2-yl)-2-methoxy-phenoxy]ethoxy]cyclobutanecarboxylic acid

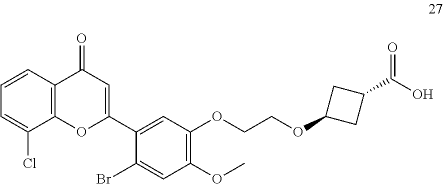 [SMILES: COc1cc(Br)c(-c2cc(=O)c3cccc(Cl)c3o2)cc1OCCO[C@H]1C[C@H](C(=O)O)C1]

### Step 1. Synthesis of methyl 3-[2-[4-bromo-5-(8-chloro-4-oxo-chromen-2-yl)-2-methoxy-phenoxy]ethoxy]cyclobutanecarboxylate

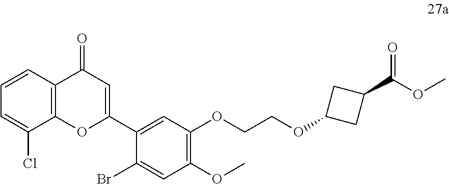 [SMILES: COc1cc(Br)c(-c2cc(=O)c3cccc(Cl)c3o2)cc1OCCO[C@H]1C[C@H](C(=O)OC)C1]

Compound 27a was prepared in analogy to the procedure described for the preparation of example 1 by using 2-(2-bromo-5-hydroxy-4-methoxyphenyl)-8-chloro-chromen-4-one and trans-methyl 3-[2-(p-tolylsulfonyloxy)ethoxy]cyclobutanecarboxylate as the starting material instead of 2-(4-bromo-5-hydroxy-2-methoxy-phenyl)-8-chloro-chromen-4-one (compound 1c) and cis-tert-butyl 3-[2-(p-tolylsulfonyloxy)ethoxy]cyclobutanecarboxylate (Intermediate AP) in Step 4. Methyl 3-[2-[4-bromo-5-(8-chloro-4-oxo-chromen-2-yl)-2-methoxy-phenoxy]ethoxy]cyclobutanecarboxylate (250 mg, compound 27a) was obtained as a yellow oil and the residue was purified by preparative HPLC to give trans-methyl-3-(2-(4-bromo-5-(8-chloro-4-oxo-chromen-2-yl)-2-methoxyphenoxy)ethoxy)cyclobutane-1-carboxylate (52 mg, compound 27a) was obtained as a light yellow solid. MS obsd. (ESI⁺) [(M+H)⁺]: 538.

### Step 2. Synthesis of trans-3-[2-[4-bromo-5-(8-chloro-4-oxo-chromen-2-yl)-2-methoxyphenoxy]ethoxy]cyclobutanecarboxylic acid

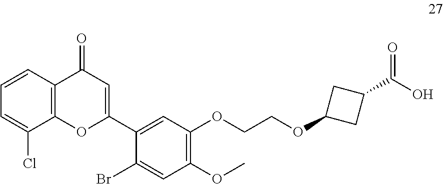 [SMILES: COc1cc(Br)c(-c2cc(=O)c3cccc(Cl)c3o2)cc1OCCO[C@H]1C[C@H](C(=O)O)C1]

Example 27 was prepared in analogy to the procedure described for the preparation of example 7 by using trans-methyl-3-(2-(4-bromo-5-(8-chloro-4-oxo-chromen-2-yl)-2-methoxyphenoxy)ethoxy)cyclobutane-1-carboxylate (52 mg, compound 28a) as the starting material instead of methyl 3-[2-chloro-5-(8-chloro-4-oxo-chromen-2-yl)-4-methoxy-phenoxy]cyclobutanecarboxylate (compound 7c) in Step 4. Trans-3-[2-[4-bromo-5-(8-chloro-4-oxo-chromen-2-yl)-2-methoxy-phenoxy]ethoxy]cyclobutanecarboxylic acid (27.4 mg, example 27) was obtained as an off-white foam. ¹H NMR (400 MHz, DMSO-d₆) δ ppm 12.16 (br d, 1H, J=0.6 Hz), 8.0-8.1 (m, 2H), 7.5-7.6 (m, 1H), 7.38 (d, 2H, J=7.9 Hz), 6.71 (s, 1H), 4.14 (br d, 3H, J=5.9 Hz), 3.89 (s, 3H), 3.65 (br s, 2H), 2.8-3.0 (m, 1H), 2.3-2.4 (m, 2H), 2.1-2.2 (m, 2H). MS obsd. (ESI⁺) [(M+H)⁺]: 524.

### Example 28

### 3-[3-(8-Chloro-4-oxo-chromen-2-yl)-2-methoxy-phenoxy]cyclobutanecarboxylic acid

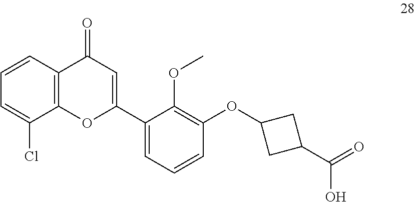 [SMILES: COc1c(OC2CC(C(=O)O)C2)cccc1-c1cc(=O)c2cccc(Cl)c2o1]

### Step 1. Synthesis of (E)-1-(3-chloro-2-hydroxy-phenyl)-3-(3-hydroxy-2-methoxy-phenyl)prop-2-en-1-one

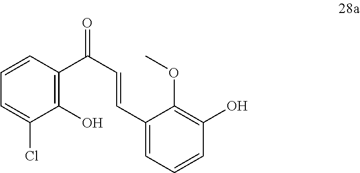 [SMILES: COc1c(O)cccc1/C=C/C(=O)c1cccc(Cl)c1O]

Compound 28a was prepared in analogy to the procedure described for the preparation of example 1 by using 3-[tert-butyl(dimethyl)silyl]oxy-2-methoxy-benzaldehyde (Intermediate AD) as the starting material instead of 4-bromo-2-methoxy-5-[(4-methoxyphenyl)methoxy]benzaldehyde (Intermediate AA) in Step 1. (E)-1-(3-chloro-2-hydroxy-phenyl)-3-(3-hydroxy-2-methoxy-phenyl)prop-2-en-1-one (1.0 g, 82% yield, compound 28a) was obtained as a yellow solid, which was used for next step without purification. MS obsd. (ESI⁺): 305.1 [(M+H)⁺], 307.1 [(M+2+H)⁺].

### Step 2. Synthesis of 8-chloro-2-(3-hydroxy-2-methoxy-phenyl)chromen-4-one

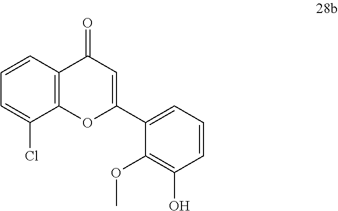 [SMILES: COc1c(O)cccc1-c1cc(=O)c2cccc(Cl)c2o1]

Compound 28b was prepared in analogy to the procedure described for the preparation of example 1 by using (E)-1-(3-chloro-2-hydroxy-phenyl)-3-(3-hydroxy-2-methoxy-phenyl)prop-2-en-1-one (compound 28a) was obtained as the starting material instead of (E)-3-[4-bromo-2-methoxy-5-[(4-methoxyphenyl)methoxy]phenyl]-1-(3-chloro-2-hydroxy-phenyl)prop-2-en-1-one (compound 1a) in Step 2. 8-Chloro-2-(3-hydroxy-2-methoxy-phenyl)chromen-4-one (180 mg, 52% yield, compound 28b) was obtained as a yellow solid. MS obsd. (ESI⁺): 303.0 [(M+H)⁺], 305.1 [(M+2+H)⁺].

### Step 3. Synthesis of methyl 3-[3-(8-chloro-4-oxo-chromen-2-yl)-2-methoxy-phenoxy]cyclobutanecarboxylate

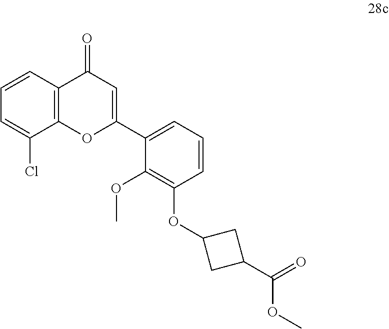 [SMILES: COC(=O)C1CC(Oc2cccc(-c3cc(=O)c4cccc(Cl)c4o3)c2OC)C1]

Compound 28c was prepared in analogy to the procedure described for the preparation of example 1 by using 8-chloro-2-(3-hydroxy-2-methoxy-phenyl)chromen-4-one (180 mg, compound 28b) and methyl 3-(p-tolylsulfonyloxy)cyclobutanecarboxylate as the starting material instead of 2-(4-bromo-5-hydroxy-2-methoxy-phenyl)-8-chloro-chromen-4-one (compound 1c) and cis-tert-butyl 3-[2-(p-tolylsulfonyloxy)ethoxy]cyclobutanecarboxylate (Intermediate AP) in Step 4. Methyl 3-[3-(8-chloro-4-oxo-chromen-2-yl)-2-methoxy-phenoxy]cyclobutanecarboxylate (65 mg, 47.43% yield, compound 28c) was obtained as a yellow oil. MS obsd. (ESI⁺): 415.1 [(M+H)⁺], 417.0 [(M+2+H)⁺].

### Step 4. Synthesis of 3-[3-(8-chloro-4-oxo-chromen-2-yl)-2-methoxy-phenoxy]cyclobutanecarboxylic acid

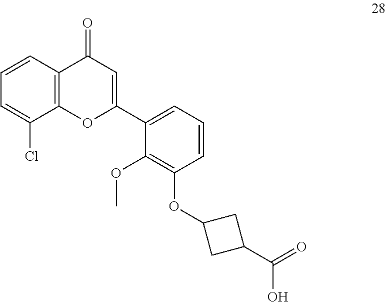 [SMILES: COc1c(OC2CC(C(=O)O)C2)cccc1-c1cc(=O)c2cccc(Cl)c2o1]

Example 28 was prepared in analogy to the procedure described for the preparation of example 7 by using methyl 3-[3-(8-chloro-4-oxo-chromen-2-yl)-2-methoxy-phenoxy]cyclobutanecarboxylate (compound 28c) was obtained as the starting material instead of methyl 3-[2-chloro-5-(8-chloro-4-oxo-chromen-2-yl)-4-methoxy-phenoxy]cyclobutanecarboxylate (compound 7c) in Step 4. 3-[3-(8-Chloro-4-oxo-chromen-2-yl)-2-methoxy-phenoxy]cyclobutanecarboxylic acid (5 mg, 10% yield, example 28) was obtained as a white solid. ¹H NMR (400 MHz, MeOD) δ ppm: 8.10 (dd, J=8.0, 1.5 Hz, 1H), 7.92 (dd, J=7.8, 1.5 Hz, 1H), 7.54 (dd, J=8.0, 1.4 Hz, 1H), 7.48 (t, J=7.9 Hz, 1H), 7.23 (t, J 8.1 Hz, 1H), 7.09 (s, 1H), 7.07 (dd, J=8.2, 1.3 Hz, 1H), 4.96-5.03 (m, 1H), 3.97 (s, 3H), 3.17-3.23 (m, 1H), 2.79 (ddd, J=13.4, 7.0, 4.3 Hz, 2H), 2.48-2.58 (m, 2H). MS obsd. (ESI⁺): 401.0 [(M+H)⁺], 403.0 [(M+2+H)⁺].

### Example 29

### Cis-3-[2-[3-(8-chloro-4-oxo-chromen-2-yl)-2-methoxy-phenoxy]ethoxy]cyclobutanecarboxylic acid

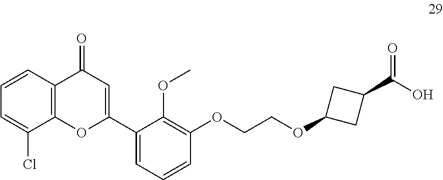 [SMILES: COc1c(OCCO[C@H]2C[C@@H](C(=O)O)C2)cccc1-c1cc(=O)c2cccc(Cl)c2o1]

### Step 1. Synthesis of tert-butyl 3-[2-[3-(8-chloro-4-oxo-chromen-2-yl)-2-methoxy-phenoxy]ethoxy]cyclobutanecarboxylate

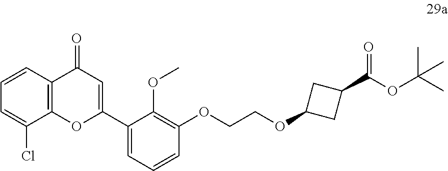 [SMILES: COc1c(OCCO[C@H]2C[C@@H](C(=O)OC(C)(C)C)C2)cccc1-c1cc(=O)c2cccc(Cl)c2o1]

Compound 29a was prepared in analogy to the procedure described for the preparation of example 1 by using 8-chloro-2-(3-hydroxy-2-methoxy-phenyl)chromen-4-one (compound 28b) as the starting material instead of 2-(4-bromo-5-hydroxy-2-methoxy-phenyl)-8-chloro-chromen-4-one (compound 1c) in Step 4. Tert-butyl 3-[2-[3-(8-chloro-4-oxo-chromen-2-yl)-2-methoxy-phenoxy]ethoxy]cyclobutanecarboxylate (150 mg, compound 29a) was obtained as a yellow oil. MS obsd. (ESI⁺): 501.1 [(M+H)⁺], 503.1 [(M+2+H)⁺].

### Step 2. Synthesis of cis-3-[2-[3-(8-chloro-4-oxo-chromen-2-yl)-2-methoxy-phenoxy]ethoxy]cyclobutanecarboxylic acid

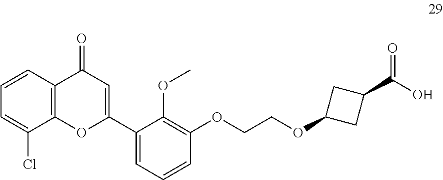 [SMILES: COc1c(OCCO[C@H]2C[C@@H](C(=O)O)C2)cccc1-c1cc(=O)c2cccc(Cl)c2o1]

Example 29 was prepared in analogy to the procedure described for the preparation of example 1 by using tert-butyl 3-[2-[3-(8-chloro-4-oxo-chromen-2-yl)-2-methoxy-phenoxy]ethoxy]cyclobutanecarboxylate (compound 29a) was obtained as the starting material instead of tert-butyl 3-[2-[2-bromo-5-(8-chloro-4-oxo-chromen-2-yl)-4-methoxy-phenoxy]ethoxy]cyclobutanecarboxylate (compound 1d) in Step 5. Cis-3-[2-[3-(8-Chloro-4-oxo-chromen-2-yl)-2-methoxy-phenoxy]ethoxy]cyclobutanecarboxylic acid (80 mg, 0.180 mmol, 60% yield, example 29) was obtained as a white solid. ¹H NMR (400 MHz, MeOD) δ ppm: 8.09 (dd, J=8.0, 1.4 Hz, 1H), 7.92 (dd, J=7.8, 1.5 Hz, 1H), 7.55 (dd, J=7.5, 1.9 Hz, 1H), 7.47 (t, J=7.9 Hz, 1H), 7.22-7.30 (m, 2H), 7.11 (s, 1H), 4.19-4.25 (m, 2H), 4.03-4.08 (m, 1H), 3.99 (s, 3H), 3.76-3.82 (m, 2H), 2.67 (dd, J=16.8, 8.8 Hz, 1H), 2.55 (ddd, J=14.8, 7.4, 2.7 Hz, 2H), 2.12-2.21 (m, 2H). MS obsd. (ESI⁺): 445.1 [(M+H)⁺], 447.1 [(M+2+H)⁺].

### Example 30

### 3-[3-(8-Chloro-4-oxo-chromen-2-yl)-4-methoxy-phenoxy]cyclobutanecarboxylic acid

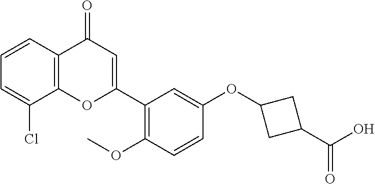 [SMILES: COc1ccc(OC2CC(C(=O)O)C2)cc1-c1cc(=O)c2cccc(Cl)c2o1]

### 30 Step 1. Synthesis of (E)-1-(3-chloro-2-hydroxy-phenyl)-3-(4-hydroxy-2-methoxy-phenyl)prop-2-en-1-one

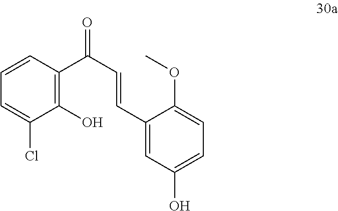 [SMILES: COc1ccc(O)cc1/C=C/C(=O)c1cccc(Cl)c1O]

Compound 30a was prepared in analogy to the procedure described for the preparation of example 1 by using 5-hydroxy-2-methoxy-benzaldehyde (Intermediate AE) as the starting material instead of 4-bromo-2-methoxy-5-[(4-methoxyphenyl)methoxy]benzaldehyde (Intermediate AA) in Step 1. (E)-1-(3-chloro-2-hydroxy-phenyl)-3-(4-hydroxy-2-methoxy-phenyl)prop-2-en-1-one (300 mg, 28% yield, compound 30a) was obtained as a yellow oil. MS obsd. (ESI⁺): 305.1 [(M+H)⁺], 307.1 [(M+2+H)⁺].

### Step 2. Synthesis of 8-chloro-2-(5-hydroxy-2-methoxy-phenyl)chromen-4-one

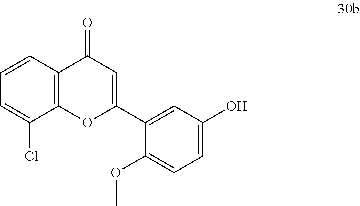 [SMILES: COc1ccc(O)cc1-c1cc(=O)c2cccc(Cl)c2o1]

Compound 30b was prepared in analogy to the procedure described for the preparation of example 1 by using (E)-1-(3-chloro-2-hydroxy-phenyl)-3-(4-hydroxy-2-methoxy-phenyl)prop-2-en-1-one (compound 30a) as the starting material instead of (E)-3-[4-bromo-2-methoxy-5-[(4-methoxyphenyl)methoxy]phenyl]-1-(3-chloro-2-hydroxy-phenyl)prop-2-en-1-one (compound 1a) in Step 2. 8-Chloro-2-(5-hydroxy-2-methoxy-phenyl)chromen-4-one (170 mg, 67% yield, compound 31b) was obtained as a yellow solid. MS obsd. (ESI⁺): 303.1 [(M+H)⁺], 305.0 [(M+2+H)⁺].

### Step 3. Synthesis of methyl 3-[3-(8-chloro-4-oxo-chromen-2-yl)-4-methoxy-phenoxy]cyclobutanecarboxylate

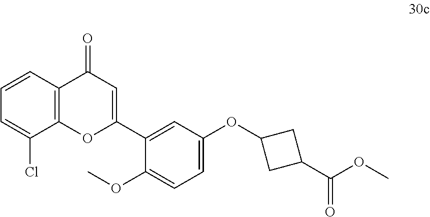 [SMILES: COC(=O)C1CC(Oc2ccc(OC)c(-c3cc(=O)c4cccc(Cl)c4o3)c2)C1]

Compound 30c was prepared in analogy to the procedure described for the preparation of example 1 by using 8-chloro-2-(5-hydroxy-2-methoxy-phenyl)chromen-4-one (compound 30b) and methyl 3-(p-tolylsulfonyloxy)cyclobutanecarboxylate as the starting material instead of 2-(4-bromo-5-hydroxy-2-methoxy-phenyl)-8-chloro-chromen-4-one (compound 1c) and cis-tert-butyl 3-[2-(p-tolylsulfonyloxy)ethoxy]cyclobutanecarboxylate (Intermediate AP) in Step 4. Methyl 3-[3-(8-chloro-4-oxo-chromen-2-yl)-4-methoxy-phenoxy]cyclobutanecarboxylate (100 mg, 38% yield, compound 30c) was obtained as a yellow solid. MS obsd. (ESI⁺): 415.1 [(M+H)⁺], 417.1 [(M+2+H)⁺].

### Step 4. Synthesis of 3-[3-(8-chloro-4-oxo-chromen-2-yl)-4-methoxy-phenoxy]cyclobutanecarboxylic acid

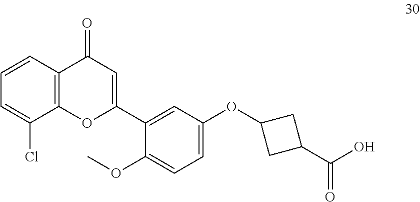 [SMILES: COc1ccc(OC2CC(C(=O)O)C2)cc1-c1cc(=O)c2cccc(Cl)c2o1]

Compound 30 was prepared in analogy to the procedure described for the preparation of example 7 by using methyl 3-[3-(8-chloro-4-oxo-chromen-2-yl)-4-methoxy-phenoxy]cyclobutanecarboxylate (compound 30c) was obtained as the starting material instead of methyl 3-[2-chloro-5-(8-chloro-4-oxo-chromen-2-yl)-4-methoxy-phenoxy]cyclobutanecarboxylate (compound 7c) in Step 4. 3-[3-(8-Chloro-4-oxo-chromen-2-yl)-4-methoxy-phenoxy]cyclobutanecarboxylic acid (53 mg, 67% yield, example 30) was obtained as a yellow solid. ¹H NMR (400 MHz, DMSO-d₆) δ ppm: 12.34 (s, 1H), 8.01 (t, J=7.4 Hz, 2H), 7.48-7.55 (m, 2H), 7.23 (d, J=8.8 Hz, 2H), 7.16 (s, 1H), 7.10 (d, J=8.9 Hz, 1H), 4.83-4.92 (m, 1H), 3.92 (s, 3H), 3.02-3.15 (m, 1H), 2.65-2.75 (m, 2H), 2.25-2.39 (m, 2H). MS obsd. (ESI⁺): 401.0 [(M+H)⁺], 403.0 [(M+2+H)⁺].

### Example 31

### Cis-3-[2-[3-(8-Chloro-4-oxo-chromen-2-yl)-4-methoxy-phenoxy]ethoxy]cyclobutanecarboxylic acid

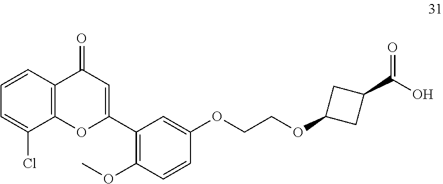 [SMILES: COc1ccc(OCCO[C@H]2C[C@@H](C(=O)O)C2)cc1-c1cc(=O)c2cccc(Cl)c2o1]

### Step 1. Synthesis of cis-tert-butyl 3-[2-[3-(8-chloro-4-oxo-chromen-2-yl)-4-methoxy-phenoxy]ethoxy]cyclobutanecarboxylate

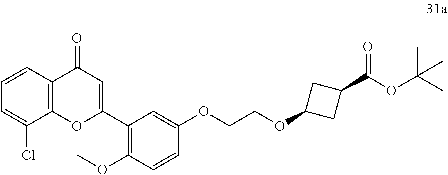 [SMILES: COc1ccc(OCCO[C@H]2C[C@@H](C(=O)OC(C)(C)C)C2)cc1-c1cc(=O)c2cccc(Cl)c2o1]

Compound 31a was prepared in analogy to the procedure described for the preparation of example 1 by using 8-chloro-2-(5-hydroxy-2-methoxy-phenyl)chromen-4-one (compound 30b) as the starting material instead of 2-(4-bromo-5-hydroxy-2-methoxy-phenyl)-8-chloro-chromen-4-one (compound 1c) in Step 4. cis-tert-Butyl 3-[2-[3-(8-chloro-4-oxo-chromen-2-yl)-4-methoxy-phenoxy]ethoxy]cyclobutanecarboxylate (90 mg, 80% yield, compound 31a) was obtained as a yellow oil. MS obsd. (ESI⁺): 501.2 [(M+H)⁺], 503.1 [(M+2+H)⁺].

### Step 2. Synthesis of cis-3-[2-[3-(8-chloro-4-oxo-chromen-2-yl)-4-methoxy-phenoxy]ethoxy]cyclobutanecarboxylic acid

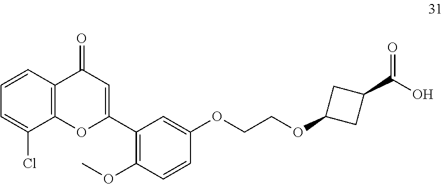 [SMILES: COc1ccc(OCCO[C@H]2C[C@@H](C(=O)O)C2)cc1-c1cc(=O)c2cccc(Cl)c2o1]

Example 31 was prepared in analogy to the procedure described for the preparation of example 1 by using tert-butyl 3-[2-[3-(8-chloro-4-oxo-chromen-2-yl)-4-methoxy-phenoxy]ethoxy]cyclobutanecarboxylate (compound 31a) as the starting material instead of tert-butyl 3-[2-[2-bromo-5-(8-chloro-4-oxo-chromen-2-yl)-4-methoxy-phenoxy]ethoxy]cyclobutanecarboxylate (compound 1d) in Step 5. Cis-3-[2-[3-(8-chloro-4-oxo-chromen-2-yl)-4-methoxy-phenoxy]ethoxy]cyclobutanecarboxylic acid (52.5 mg, 65% yield, example 31) was obtained as a yellow solid. ¹H NMR (400 MHz, MeOD) δ ppm: 7.99-8.02 (m, 1H), 7.82-7.90 (m, 1H), 7.60-7.70 (m, 1H), 7.40-7.50 (m, 1H), 7.20-7.31 (m, 1H), 7.10-7.15 (m, 2H), 4.10-4.15 (m, 2H), 3.95-4.10 (m, 1H), 3.94 (d, J=2.5 Hz, 3H), 3.71-3.78 (m, 2H), 2.66 (dd, J=16.8, 8.8 Hz, 1H), 2.50-2.59 (m, 2H), 2.11-2.22 (m, 2H). MS obsd. (ESI⁺): 445.1 [(M+H)⁺], 447.1 [(M+2+H)⁺].

### Example 32

### 2-[3-(8-Chloro-4-oxo-chromen-2-yl)-4-methoxy-phenoxy]acetic acid

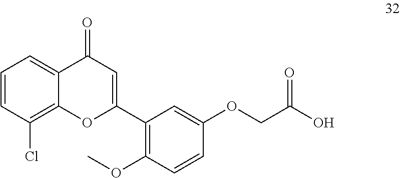 [SMILES: COc1ccc(OCC(=O)O)cc1-c1cc(=O)c2cccc(Cl)c2o1]

### Step 1. Synthesis of tert-butyl 2-[3-(8-chloro-4-oxo-chromen-2-yl)-4-methoxy-phenoxy]acetate

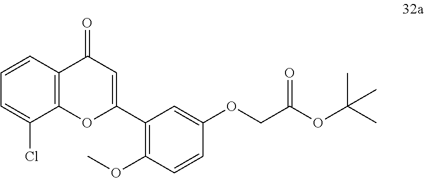 [SMILES: COc1ccc(OCC(=O)OC(C)(C)C)cc1-c1cc(=O)c2cccc(Cl)c2o1]

Compound 32a was prepared in analogy to the procedure described for the preparation of example 1 by using 8-chloro-2-(5-hydroxy-2-methoxy-phenyl)chromen-4-one (compound 31b) and tert-butyl bromoacetate as the starting material instead of 2-(4-bromo-5-hydroxy-2-methoxy-phenyl)-8-chloro-chromen-4-one (compound 1c) and cis-tert-butyl 3-[2-(p-tolylsulfonyloxy)ethoxy]cyclobutanecarboxylate (Intermediate AP) in Step 4. Tert-butyl 2-[3-(8-chloro-4-oxo-chromen-2-yl)-4-methoxy-phenoxy]acetate (70 mg, 72% yield, compound 32a) was obtained as a yellow solid. MS obsd. (ESI⁺): 417.1 [(M+H)⁺], 419.1 [(M+2+H)⁺].

### Step 2. Synthesis of 2-[3-(8-chloro-4-oxo-chromen-2-yl)-4-methoxy-phenoxy]acetic acid

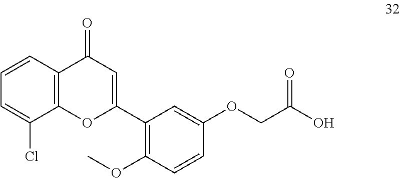 [SMILES: COc1ccc(OCC(=O)O)cc1-c1cc(=O)c2cccc(Cl)c2o1]

Example 32 was prepared in analogy to the procedure described for the preparation of example 1 by using tert-butyl 2-[3-(8-chloro-4-oxo-chromen-2-yl)-4-methoxy-phenoxy]acetate (compound 32a) was obtained as the starting material instead of tert-butyl 3-[2-[2-bromo-5-(8-chloro-4-oxo-chromen-2-yl)-4-methoxy-phenoxy]ethoxy]cyclobutanecarboxylate (compound 1d) in Step 5. 2-[3-(8-Chloro-4-oxo-chromen-2-yl)-4-methoxy-phenoxy]acetic acid (45 mg, 74% yield, example 32) was obtained as a yellow solid. ¹H NMR (400 MHz, DMSO-d₆) δ ppm 13.06 (s, 1H), 7.97-8.04 (m, 2H), 7.47-7.55 (m, 1H), 7.21 (dt, J=9.1, 6.1 Hz, 1H), 7.12 (s, 1H), 4.73 (s, 2H), 3.92 (s, 3H). MS obsd. (ESI⁺): 361.0 [(M+H)⁺], 363.0 [(M+2+H)⁺].

### Example 33

### 3-[3-(8-Chloro-4-oxo-chromen-2-yl)-4-methoxy-phenoxy]propanoic acid

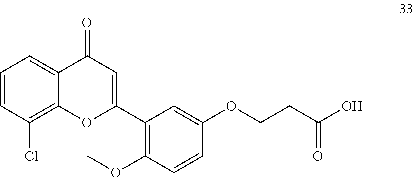 [SMILES: COc1ccc(OCCC(=O)O)cc1-c1cc(=O)c2cccc(Cl)c2o1]

Example 33 was prepared in analogy to the procedure described for the preparation of example 2 by using 8-chloro-2-(5-hydroxy-2-methoxy-phenyl)chromen-4-one (compound 31b) as the starting material instead of 2-(4-bromo-5-hydroxy-2-methoxy-phenyl)-8-chloro-chromen-4-one (compound 1c). 3-[3-(8-Chloro-4-oxo-chromen-2-yl)-4-methoxy-phenoxy]propanoic acid (6 mg, 3% yield, example 33) was obtained as a yellow solid. ¹H NMR (400 MHz, DMSO-d₆) δ ppm 12.35 (s, 1H), 7.99-7.82 (m, 2H), 7.54 (d, J=2.1 Hz, 1H), 7.50 (t, J=7.9 Hz, 1H), 7.17-7.26 (m, 2H), 7.10 (s, 1H), 4.22 (t, J=6.1 Hz, 2H), 3.91 (s, 3H), 2.72 (t, J=6.0 Hz, 2H). MS obsd. (ESI⁺): 375.1 [(M+H)⁺], 377.1 [(M+2+H)⁺].

### Example 34

### Cis-3-[2-[6-bromo-3-(8-chloro-4-oxo-chromen-2-yl)-2-methoxy-phenoxy]ethoxy]cyclobutanecarboxylic acid

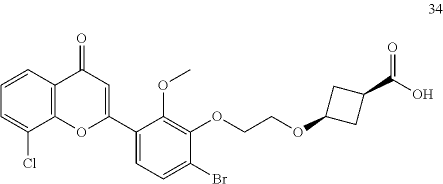 [SMILES: COc1c(-c2cc(=O)c3cccc(Cl)c3o2)ccc(Br)c1OCCO[C@H]1C[C@@H](C(=O)O)C1]

### Step 1. Synthesis of (E)-3-(4-bromo-3-hydroxy-2-methoxy-phenyl)-1-(3-chloro-2-hydroxy-phenyl)prop-2-en-1-one

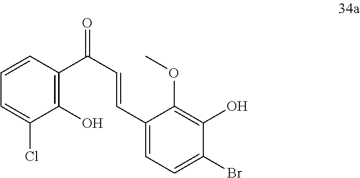 [SMILES: COc1c(/C=C/C(=O)c2cccc(Cl)c2O)ccc(Br)c1O]

Compound 34a was prepared in analogy to the procedure described for the preparation of example 1 by using 4-bromo-3-hydroxy-2-methoxy-benzaldehyde (Intermediate AF) as the starting material instead of 4-bromo-2-methoxy-5-[(4-methoxyphenyl)methoxy]benzaldehyde (Intermediate AA) in Step 1. (E)-3-(4-Bromo-3-hydroxy-2-methoxy-phenyl)-1-(3-chloro-2-hydroxy-phenyl)prop-2-en-1-one (300 mg, 21% yield, compound 34a) was obtained as a yellow solid. MS obsd. (ESI⁺): 383.1 [(M+H)⁺], 385.1 [(M+2+H)⁺].

### Step 2. Synthesis of 2-(4-bromo-3-hydroxy-2-methoxy-phenyl)-8-chloro-chromen-4-one

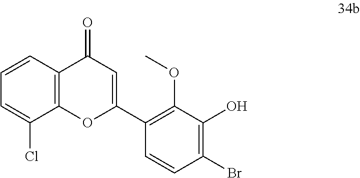 [SMILES: COc1c(-c2cc(=O)c3cccc(Cl)c3o2)ccc(Br)c1O]

Compound 34b was prepared in analogy to the procedure described for the preparation of example 1 by using (E)-3-(4-bromo-3-hydroxy-2-methoxy-phenyl)-1-(3-chloro-2-hydroxy-phenyl)prop-2-en-1-one (compound 34a) as the starting material instead of (E)-3-[4-bromo-2-methoxy-5-[(4-methoxyphenyl)methoxy]phenyl]-1-(3-chloro-2-hydroxy-phenyl)prop-2-en-1-one (compound 1a) in Step 2. 2-(4-Bromo-3-hydroxy-2-methoxy-phenyl)-8-chloro-chromen-4-one (180 mg, 80% yield, compound 34b) was obtained as a yellow solid. MS obsd. (ESI⁺): 380.9[(M+H)⁺], 382.9[(M+2+H)⁺].

### Step 3. Synthesis of cis-tert-butyl 3-[2-[6-bromo-3-(8-chloro-4-oxo-chromen-2-yl)-2-methoxy-phenoxy]ethoxy]cyclobutanecarboxylate

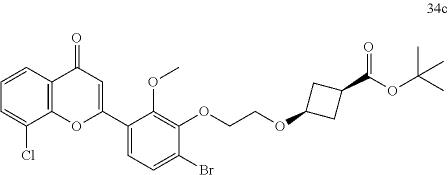 [SMILES: COc1c(-c2cc(=O)c3cccc(Cl)c3o2)ccc(Br)c1OCCO[C@H]1C[C@@H](C(=O)OC(C)(C)C)C1]

Compound 34c was prepared in analogy to the procedure described for the preparation of example 1 by 2-(4-bromo-3-hydroxy-2-methoxy-phenyl)-8-chloro-chromen-4-one (180 mg, 80% yield, compound 34b) as the starting material instead of 2-(4-bromo-5-hydroxy-2-methoxy-phenyl)-8-chloro-chromen-4-one (compound 1c) in Step 4. Cis-tert-Butyl 3-[2-[6-bromo-3-(8-chloro-4-oxo-chromen-2-yl)-2-methoxy-phenoxy]ethoxy]cyclobutanecarboxylate (100 mg, 71% yield, compound 34c) was obtained as a white solid. MS obsd. (ESI⁺): 579.1 [(M+H)⁺], 581.1 [(M+2+H)⁺].

### Step 4. Synthesis of cis-3-[2-[6-bromo-3-(8-chloro-4-oxo-chromen-2-yl)-2-methoxy-phenoxy]ethoxy]cyclobutanecarboxylic acid

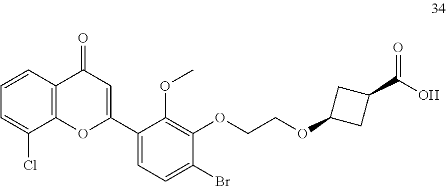 [SMILES: COc1c(-c2cc(=O)c3cccc(Cl)c3o2)ccc(Br)c1OCCO[C@H]1C[C@@H](C(=O)O)C1]

Example 34 was prepared in analogy to the procedure described for the preparation of example 1 by using cis-tert-butyl 3-[2-[6-bromo-3-(8-chloro-4-oxo-chromen-2-yl)-2-methoxy-phenoxy]ethoxy]cyclobutanecarboxylate (compound 34c) as the starting material instead of tert-butyl 3-[2-[2-bromo-5-(8-chloro-4-oxo-chromen-2-yl)-4-methoxy-phenoxy]ethoxy]cyclobutanecarboxylate (compound 1d) in Step 5. Cis-3-[2-[6-bromo-3-(8-chloro-4-oxo-chromen-2-yl)-2-methoxy-phenoxy]ethoxy]cyclobutanecarboxylic acid (58 mg, 64% yield, example 34) was obtained as a white solid. ¹H NMR (400 MHz, DMSO-d₆) δ ppm 12.08 (s, 1H), 8.03 (d, J=8.2 Hz, 2H), 7.64 (d, J=8.6 Hz, 1H), 7.50-7.61 (m, 2H), 6.93 (s, 1H), 4.11-4.19 (m, 2H), 3.91-4.02 (m, 4H), 3.62-3.72 (m, 2H), 2.57-2.67 (m, 1H), 2.38-2.49 (m, 2H), 1.95-2.06 (m, 2H). MS obsd. (ESI⁺): 523.0 [(M+H)⁺], 525.0 [(M+2+H)⁺].

### Example 35

### 3-[6-Bromo-3-(8-chloro-4-oxo-chromen-2-yl)-2-methoxy-phenoxy]cyclobutanecarboxylic acid

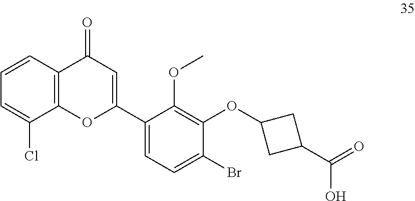 [SMILES: COc1c(-c2cc(=O)c3cccc(Cl)c3o2)ccc(Br)c1OC1CC(C(=O)O)C1]

### Step 1. Synthesis of methyl 3-[6-bromo-3-(8-chloro-4-oxo-chromen-2-yl)-2-methoxy-phenoxy]cyclobutanecarboxylate

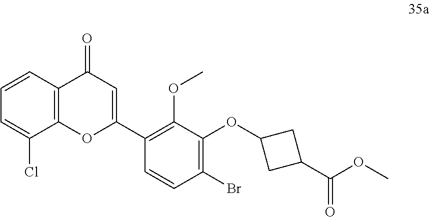 [SMILES: COC(=O)C1CC(Oc2c(Br)ccc(-c3cc(=O)c4cccc(Cl)c4o3)c2OC)C1]

Compound 35a was prepared in analogy to the procedure described for the preparation of example 1 by using 2-(4-bromo-3-hydroxy-2-methoxy-phenyl)-8-chloro-chromen-4-one (compound 34b) and methyl 3-(p-tolylsulfonyloxy)cyclobutanecarboxylate as the starting material instead of 2-(4-bromo-5-hydroxy-2-methoxy-phenyl)-8-chloro-chromen-4-one (compound 1c) and cis-tert-butyl 3-[2-(p-tolylsulfonyloxy)ethoxy]cyclobutanecarboxylate in Step 4. Methyl 3-[6-bromo-3-(8-chloro-4-oxo-chromen-2-yl)-2-methoxy-phenoxy]cyclobutanecarboxylate (100 mg, 74% yield, compound 35a) was obtained as a yellow solid. MS obsd. (ESI⁺): 493.0 [(M+H)⁺], 495.0 [(M+2+H)⁺].

### Step 2. Synthesis of 3-[6-bromo-3-(8-chloro-4-oxo-chromen-2-yl)-2-methoxy-phenoxy]cyclobutanecarboxylic acid

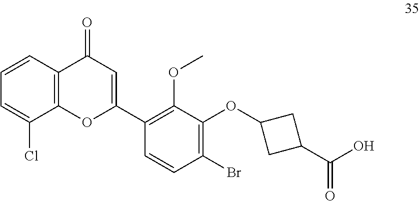 [SMILES: COc1c(-c2cc(=O)c3cccc(Cl)c3o2)ccc(Br)c1OC1CC(C(=O)O)C1]

Example 35 was prepared in analogy to the procedure described for the preparation of example 7 by using methyl 3-[6-bromo-3-(8-chloro-4-oxo-chromen-2-yl)-2-methoxy-phenoxy]cyclobutanecarboxylate (compound 35a) as the starting material instead of methyl 3-[2-chloro-5-(8-chloro-4-oxo-chromen-2-yl)-4-methoxy-phenoxy]cyclobutanecarboxylate (compound 7c) in Step 4. 3-[6-Bromo-3-(8-chloro-4-oxo-chromen-2-yl)-2-methoxy-phenoxy]cyclobutanecarboxylic acid (55 mg, 49% yield, example 35) obtained as an off-white solid. ¹H NMR (400 MHz, MeOD) δ ppm: 8.09 (dd, J=8.0, 1.5 Hz, 1H), 7.92 (dd, J=7.8, 1.5 Hz, 1H), 7.67 (d, J=8.7 Hz, 1H), 7.58 (d, J=8.7 Hz, 1H), 7.48 (t, J=7.9 Hz, 1H), 7.12 (s, 1H), 4.95-5.05 (m, 1H), 3.95 (s, 3H), 3.12-3.19 (m, 1H), 2.56-2.71 (m, 4H). MS obsd. (ESI⁺): 479.0 [(M+H)⁺], 481.0 [(M+2+H)⁺].

### Example 36

### 3-[3-(8-Chloro-4-oxo-chromen-2-yl)-2-methoxy-6-methyl-phenoxy]cyclobutanecarboxylic acid

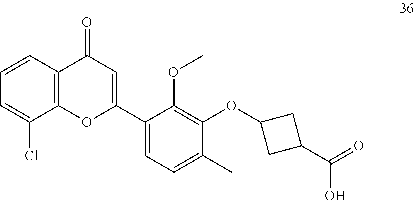 [SMILES: COc1c(-c2cc(=O)c3cccc(Cl)c3o2)ccc(C)c1OC1CC(C(=O)O)C1]

### Step 1. Synthesis of (E)-1-(3-chloro-2-hydroxy-phenyl)-3-[2-methoxy-3-(methoxymethoxy)-4-methyl-phenyl]prop-2-en-1-one

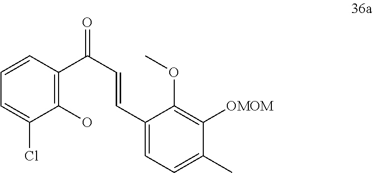

Compound 36a was prepared in analogy to the procedure described for the preparation of example 1 by using 2-methoxy-3-(methoxymethoxy)-4-methyl-benzaldehyde (Intermediate AG) as the starting material instead of 4-bromo-2-methoxy-5-[(4-methoxyphenyl)methoxy]benzaldehyde (Intermediate AA) in Step 1. (E)-1-(3-chloro-2-hydroxy-phenyl)-3-[2-methoxy-3-(methoxymethoxy)-4-methyl-phenyl]prop-2-en-1-one (180 mg, compound 36a) was obtained as a yellow solid, which was used for next step without purification. MS obsd. (ESI⁺): 363.1 [(M+H)⁺], 365.2 [(M+2+H)⁺].

### Step 2. Synthesis of 8-chloro-2-(3-hydroxy-2-methoxy-4-methyl-phenyl)chromen-4-one

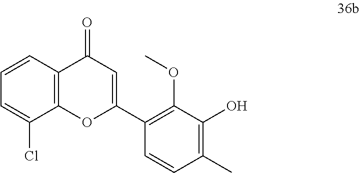 [SMILES: COc1c(-c2cc(=O)c3cccc(Cl)c3o2)ccc(C)c1O]

Compound 36b was prepared in analogy to the procedure described for the preparation of example 1 by using (E)-1-(3-chloro-2-hydroxy-phenyl)-3-[2-methoxy-3-(methoxymethoxy)-4-methyl-phenyl]prop-2-en-1-one (compound 36a) as the starting material instead of (E)-3-[4-bromo-2-methoxy-5-[(4-methoxyphenyl)methoxy]phenyl]-1-(3-chloro-2-hydroxy-phenyl)prop-2-en-1-one (compound 1a) in Step 2. 8-Chloro-2-(3-hydroxy-2-methoxy-4-methyl-phenyl)chromen-4-one (100 mg, 42% yield, compound 36b) was obtained as a brown solid. MS obsd. (ESI⁺): 317.1 [(M+H)⁺], 319.0 [(M+2+H)⁺].

### Step 3. Synthesis of methyl 3-[3-(8-chloro-4-oxo-chromen-2-yl)-2-methoxy-6-methyl-phenoxy]cyclobutanecarboxylate

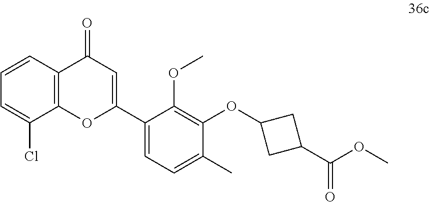 [SMILES: COC(=O)C1CC(Oc2c(C)ccc(-c3cc(=O)c4cccc(Cl)c4o3)c2OC)C1]

Compound 36c was prepared in analogy to the procedure described for the preparation of example 1 by using 8-chloro-2-(3-hydroxy-2-methoxy-4-methyl-phenyl)chromen-4-one (compound 36b) and methyl 3-(p-tolylsulfonyloxy)cyclobutanecarboxylate as the starting material instead of 2-(4-bromo-5-hydroxy-2-methoxy-phenyl)-8-chloro-chromen-4-one (compound 1c) and cis-tert-butyl 3-[2-(p-tolylsulfonyloxy)ethoxy]cyclobutanecarboxylate (Intermediate AP) in Step 4. Methyl 3-[3-(8-chloro-4-oxo-chromen-2-yl)-2-methoxy-6-methyl-phenoxy]cyclobutanecarboxylate (70 mg, 77% yield, compound 36c) was obtained as a yellow oil. MS obsd. (ESI⁺): 429.2 [(M+H)⁺], 431.2 [(M+2+H)⁺].

### Step 4. Synthesis of 3-[3-(8-chloro-4-oxo-chromen-2-yl)-2-methoxy-6-methyl-phenoxy]cyclobutanecarboxylic acid

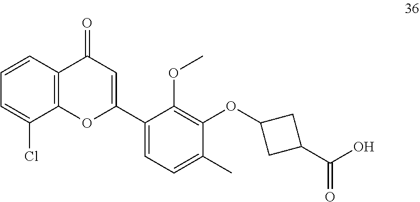 [SMILES: COc1c(-c2cc(=O)c3cccc(Cl)c3o2)ccc(C)c1OC1CC(C(=O)O)C1]

Example 36 was prepared in analogy to the procedure described for the preparation of example 7 by using methyl 3-[3-(8-chloro-4-oxo-chromen-2-yl)-2-methoxy-6-methyl-phenoxy]cyclobutanecarboxylate (70 mg, compound 36c) as the starting material instead of methyl 3-[2-chloro-5-(8-chloro-4-oxo-chromen-2-yl)-4-methoxy-phenoxy]cyclobutanecarboxylate (compound 7c) in Step 4. 3-[3-(8-Chloro-4-oxo-chromen-2-yl)-2-methoxy-6-methyl-phenoxy]cyclobutanecarboxylic acid (20 mg, 29% yield, example 36) was obtained as a white solid. ¹H NMR (400 MHz, MeOD) δ ppm: 8.09 (dd, J=8.0, 1.5 Hz, 1H), 7.91 (dd, J=7.8, 1.5 Hz, 1H), 7.67 (d, J=8.2 Hz, 1H), 7.47 (t, J=7.9 Hz, 1H), 7.17 (d, J=8.9 Hz, 2H), 4.85-4.89 (m, 1H), 3.89 (s, 3H), 3.09-3.15 (m, 1H), 2.55-2.65 (m, 4H), 2.36 (s, 3H). MS obsd. (ESI⁺): 415.1 [(M+H)⁺], 417.1 [(M+2+H)⁺].

### Example 37

### 2-[6-Chloro-3-(8-chloro-4-oxo-chromen-2-yl)-2-methoxy-phenoxy]acetic acid

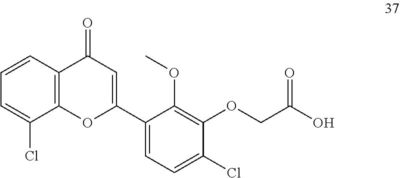 [SMILES: COc1c(-c2cc(=O)c3cccc(Cl)c3o2)ccc(Cl)c1OCC(=O)O]

### Step 1. Synthesis of tert-butyl 2-[6-chloro-3-(8-chloro-4-oxo-chromen-2-yl)-2-methoxy-phenoxy]acetate

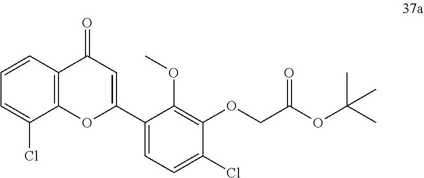 [SMILES: COc1c(-c2cc(=O)c3cccc(Cl)c3o2)ccc(Cl)c1OCC(=O)OC(C)(C)C]

Compound 37a was prepared in analogy to the procedure described for the preparation of example 1 by using 8-chloro-2-(4-chloro-3-hydroxy-2-methoxy-phenyl)chromen-4-one and tert-butyl bromoacetate as the starting material instead of 2-(4-bromo-5-hydroxy-2-methoxy-phenyl)-8-chloro-chromen-4-one (compound 1c) and cis-tert-butyl 3-[2-(p-tolylsulfonyloxy)ethoxy]cyclobutanecarboxylate in Step 4. Tert-butyl 2-[6-chloro-3-(8-chloro-4-oxo-chromen-2-yl)-2-methoxy-phenoxy]acetate (65 mg, 46% yield, compound 37a) was obtained as a yellow oil. MS obsd. (ESI⁺): 451.0 [(M+H)⁺], 453.1 [(M+2+H)⁺].

### Step 2. Synthesis of 2-[6-chloro-3-(8-chloro-4-oxo-chromen-2-yl)-2-methoxy-phenoxy]acetic acid

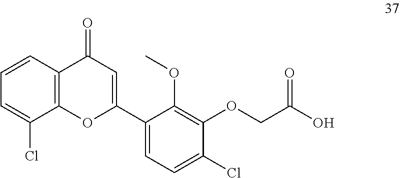 [SMILES: COc1c(-c2cc(=O)c3cccc(Cl)c3o2)ccc(Cl)c1OCC(=O)O]

Example 37 was prepared in analogy to the procedure described for the preparation of example 1 by using tert-butyl 2-[6-chloro-3-(8-chloro-4-oxo-chromen-2-yl)-2-methoxy-phenoxy]acetate (65 mg, compound 37a) was obtained as the starting material instead of tert-butyl 3-[2-[2-bromo-5-(8-chloro-4-oxo-chromen-2-yl)-4-methoxy-phenoxy]ethoxy]cyclobutanecarboxylate (compound 1d) in Step 5. 2-[6-Chloro-3-(8-chloro-4-oxo-chromen-2-yl)-2-methoxy-phenoxy]acetic acid (50 mg, 87% yield, Example 37) was obtained as a white solid. ¹H NMR (400 MHz, DMSO-d₆) δ ppm 13.02 (br s, 1H), 8.02-8.10 (m, 2H), 7.56 (t, J=7.9 Hz, 1H), 7.37 (d, J=9.0 Hz, 1H), 7.25 (d, J=9.1 Hz, 1H), 6.66 (s, 1H), 4.87 (s, 2H), 3.88 (s, 3H). MS obsd. (ESI⁺): 395.0 [(M+H)⁺], 397.1 [(M+2+H)⁺].

### Example 38

### 2-[3-(8-Chloro-4-oxo-chromen-2-yl)-2,6-dimethoxy-phenoxy]acetic acid

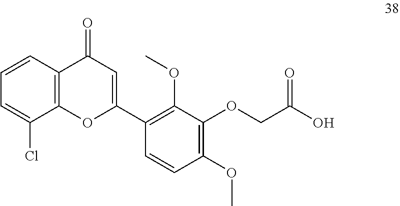 [SMILES: COc1ccc(-c2cc(=O)c3cccc(Cl)c3o2)c(OC)c1OCC(=O)O]

### Step 1. Synthesis of (E)-1-(3-chloro-2-hydroxy-phenyl)-3-[2,4-dimethoxy-3-[(4-methoxyphenyl)methoxy]phenyl]prop-n2-en-1-one

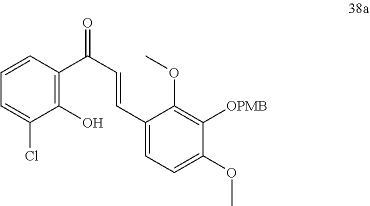

Compound 38a was prepared in analogy to the procedure described for the preparation of example 1 by using 2,4-dimethoxy-3-[(4-methoxyphenyl)methoxy]benzaldehyde (Intermediate AI) was obtained as the starting material instead of 4-bromo-2-methoxy-5-[(4-methoxyphenyl)methoxy]benzaldehyde (Intermediate AA) in Step 1. (E)-1-(3-chloro-2-hydroxy-phenyl)-3-[2,4-dimethoxy-3-[(4-methoxyphenyl)methoxy]phenyl]prop-2-en-1-one (1 g, 51% yield, compound 38a) was obtained as a yellow solid MS obsd. (ESI⁺): 455.1 [(M+H)⁺], 457.1 [(M+2+H)⁺].

### Step 2. Synthesis of 8-chloro-2-(3-hydroxy-2,4-dimethoxy-phenyl)chromen-4-one

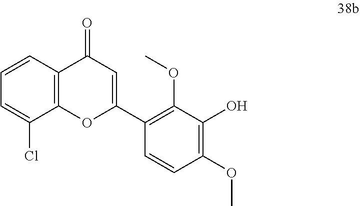 [SMILES: COc1ccc(-c2cc(=O)c3cccc(Cl)c3o2)c(OC)c1O]

Compound 38b was prepared in analogy to the procedure described for the preparation of example 1 by using (E)-1-(3-chloro-2-hydroxy-phenyl)-3-[2,4-dimethoxy-3-[(4-methoxyphenyl)methoxy]phenyl]prop-2-en-1-one (1.0 g, compound 38a) as the starting material instead of (E)-3-[4-bromo-2-methoxy-5-[(4-methoxyphenyl)methoxy]phenyl]-1-(3-chloro-2-hydroxy-phenyl)prop-2-en-1-one (compound 1a) in Step 2. 8-chloro-2-(3-hydroxy-2,4-dimethoxy-phenyl)chromen-4-one (400 mg, 54% yield, compound 38b) was obtained as a grey solid. MS obsd. (ESI⁺): 333.1 [(M+H)⁺], 335.0 [(M+2+H)⁺].

### Step 3. Synthesis of tert-butyl 2-[3-(8-chloro-4-oxo-chromen-2-yl)-2,6-dimethoxy-phenoxy]acetate

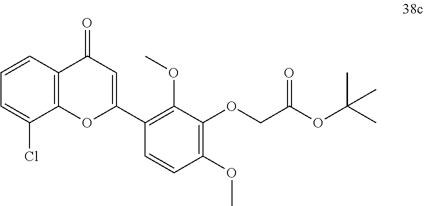 [SMILES: COc1ccc(-c2cc(=O)c3cccc(Cl)c3o2)c(OC)c1OCC(=O)OC(C)(C)C]

Compound 38c was prepared in analogy to the procedure described for the preparation of example 1 by using 8-chloro-2-(3-hydroxy-2,4-dimethoxy-phenyl)chromen-4-one (400 mg, compound 38b) and tert-butyl bromoacetate as the starting material instead of 2-(4-bromo-5-hydroxy-2-methoxy-phenyl)-8-chloro-chromen-4-one (compound 1c) and cis-tert-butyl 3-[2-(p-tolylsulfonyloxy)ethoxy]cyclobutanecarboxylate in Step 4. Tert-butyl 2-[3-(8-chloro-4-oxo-chromen-2-yl)-2,6-dimethoxy-phenoxy]acetate (100 mg, 74% yield, compound 38c) was obtained as a yellow solid. MS obsd. (ESI⁺): 447.1 [(M+H)⁺], 449.1 [(M+2+H)⁺].

### Step 4. Synthesis of 2-[3-(8-chloro-4-oxo-chromen-2-yl)-2,6-dimethoxy-phenoxy]acetic acid

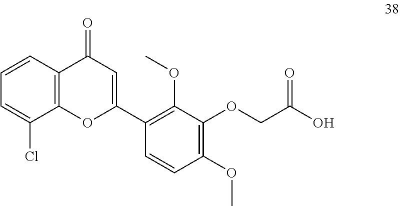 [SMILES: COc1ccc(-c2cc(=O)c3cccc(Cl)c3o2)c(OC)c1OCC(=O)O]

Example 38 was prepared in analogy to the procedure described for the preparation of example 1 by using tert-butyl 2-[3-(8-chloro-4-oxo-chromen-2-yl)-2,6-dimethoxy-phenoxy]acetate (100 mg, compound 38c) as the starting material instead of tert-butyl 3-[2-[2-bromo-5-(8-chloro-4-oxo-chromen-2-yl)-4-methoxy-phenoxy]ethoxy]cyclobutanecarboxylate (compound 1d) in Step 5. 2-[3-(8-Chloro-4-oxo-chromen-2-yl)-2,6-dimethoxy-phenoxy]acetic acid (68.9 mg, 85% yield, Example 38) was obtained as a yellow solid. ¹H NMR (400 MHz, DMSO-d₆) δ ppm 13.02 (br, 1H), 8.02 (d, J=8.0 Hz, 2H), 7.67 (d, J=8.0 Hz, 1H), 7.50 (t, J=8.0 Hz, 1H), 7.09 (d, J=8.0 Hz, 1H), 6.89 (s, 1H), 4.60 (s, 2H), 3.95 (s, 3H), 3.90 (s, 3H). MS obsd. (ESI⁺): 391.0 [(M+H)⁺], 393.0 [(M+2+H)⁺].

### Example 39

### 3-[2-Bromo-5-(8-chloro-4-oxo-chromen-2-yl)-4-ethoxy-phenoxy]propanoic acid

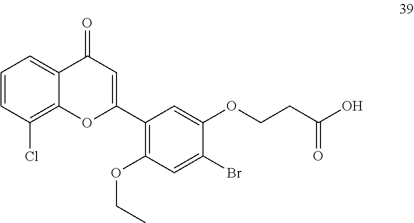 [SMILES: CCOc1cc(Br)c(OCCC(=O)O)cc1-c1cc(=O)c2cccc(Cl)c2o1]

### Step 1. Synthesis of (E)-3-[4-bromo-2-ethoxy-5-(methoxymethoxy)phenyl]-1-(3-chloro-2-hydroxy-phenyl)prop-2-en-1-one

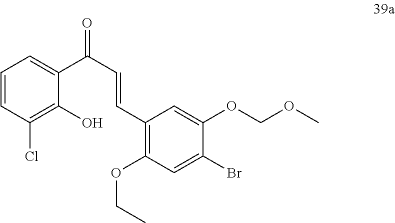 [SMILES: CCOc1cc(Br)c(OCOC)cc1/C=C/C(=O)c1cccc(Cl)c1O]

Compound 39a was prepared in analogy to the procedure described for the preparation of example 1 by using 4-bromo-2-ethoxy-5-(methoxymethoxy)benzaldehyde (Intermediate AJ) as the starting material instead of 4-bromo-2-methoxy-5-[(4-methoxyphenyl)methoxy]benzaldehyde (Intermediate AA) in Step 1. (E)-3-[4-Bromo-2-ethoxy-5-(methoxymethoxy)phenyl]-1-(3-chloro-2-hydroxy-phenyl)prop-2-en-1-one (100 mg, 93% yield, compound 39 a) was obtained as a white solid. MS obsd. (ESI⁺): 441.0 [(M+H)⁺], 443.0 [(M+2+H)⁺], 445.0 [(M+4+H)⁺].

### Step 2. Synthesis of 2-(4-bromo-2-ethoxy-5-hydroxy-phenyl)-8-chloro-chromen-4-one

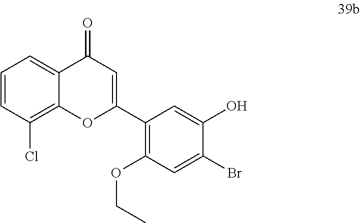 [SMILES: CCOc1cc(Br)c(O)cc1-c1cc(=O)c2cccc(Cl)c2o1]

Compound 39b was prepared in analogy to the procedure described for the preparation of example 1 by using (E)-3-[4-bromo-2-ethoxy-5-(methoxymethoxy)phenyl]-1-(3-chloro-2-hydroxy-phenyl)prop-2-en-1-one (100 mg, compound 39 a) as the starting material instead of (E)-3-[4-bromo-2-methoxy-5-[(4-methoxyphenyl)methoxy]phenyl]-1-(3-chloro-2-hydroxy-phenyl)prop-2-en-1-one (compound 1a) in Step 2. 2-(4-bromo-2-ethoxy-5-hydroxy-phenyl)-8-chloro-chromen-4-one (90 mg, 90% yield, compound 39b) was obtained as a yellow solid. MS obsd. (ESI⁺): 395.0 [(M+H)⁺], 397.0 [(M+2+H)⁺], 398.9 [(M+4+H)⁺].

### Step 3. Synthesis of 2-[4-bromo-5-[2-(1,3-dioxolan-2-yl)ethoxy]-2-ethoxy-phenyl]-8-chloro-chromen-4-one

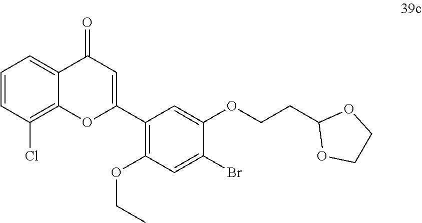 [SMILES: CCOc1cc(Br)c(OCCC2OCCO2)cc1-c1cc(=O)c2cccc(Cl)c2o1]

Compound 39c was prepared in analogy to the procedure described for the preparation of example 1 by using 2-(4-bromo-2-ethoxy-5-hydroxy-phenyl)-8-chloro-chromen-4-one (90 mg, compound 39b) and 2-(2-bromoethyl)-1,3-dioxolane as the starting material instead of 2-(4-bromo-5-hydroxy-2-methoxy-phenyl)-8-chloro-chromen-4-one (compound 1c) and cis-tert-butyl 3-[2-(p-tolylsulfonyloxy)ethoxy]cyclobutanecarboxylate in Step 4. 2-[4-Bromo-5-[2-(1,3-dioxolan-2-yl)ethoxy]-2-ethoxy-phenyl]-8-chloro-chromen-4-one (70 mg, 85%, compound 39c) was obtained as an white solid. MS obsd. (ESI⁺): 495.0 [(M+H)⁺], 497.0 [(M+2+H)⁺], 499.0 [(M+4+H)⁺].

### Step 4. Synthesis of 3-[2-bromo-5-(8-chloro-4-oxo-chromen-2-yl)-4-ethoxy-phenoxy]propanal

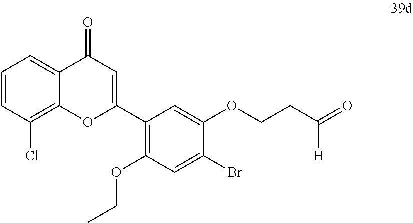 [SMILES: CCOc1cc(Br)c(OCCC=O)cc1-c1cc(=O)c2cccc(Cl)c2o1]

To a solution of 2-[4-bromo-5-[2-(1,3-dioxolan-2-yl)ethoxy]-2-ethoxy-phenyl]-8-chloro-chromen-4-one (70.0 mg) in water (0.5 mL) was added trifluoroacetic acid dropwise (2.0 mL). The mixture was stirred at 25° C. for 2 hrs. After reaction, water (500 mL) was then added to the solution. The solution was extracted with EtOAc (200 mL) three times. The combined organic layer was washed with brine (300 mL) two times, dried with MgSO₄, filtered and concentrated filtrate to afford 3-[2-bromo-5-(8-chloro-4-oxo-chromen-2-yl)-4-ethoxy-phenoxy]propanal (60 mg, 94% yield, Compound 39d) was obtained as a yellow solid. MS obsd. (ESI⁺): 451.0 [(M+H)⁺], 453.0 [(M+2+H)⁺], 455.0 [(M+4+H)⁺].

### Step 5. Synthesis of 3-[2-bromo-5-(8-chloro-4-oxo-chromen-2-yl)-4-ethoxy-phenoxy]propanoic acid

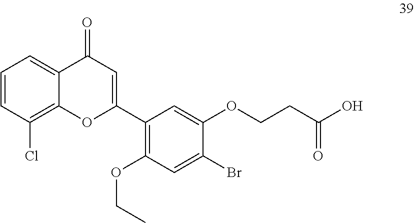 [SMILES: CCOc1cc(Br)c(OCCC(=O)O)cc1-c1cc(=O)c2cccc(Cl)c2o1]

A mixture of 3-[2-bromo-5-(8-chloro-4-oxo-chromen-2-yl)-4-ethoxy-phenoxy]propanal (60.0 mg, 0.130 mmol), 2-methyl-2-butene (83.84 mg, 1.2 mmol), chlorosyloxysodium (36.04 mg, 0.4 mmol), sodium phosphate monobasic (44.62 mg, 0.370 mmol) in 1,4-dioxane (2.51 mL) and water (0.835 mL). The pH was adjusted to around 6 by 1M HCl. The mixture was added water (400 mL). The mixture was filtered. The filter cake was washed with water (400 mL) and DCM (150 mL) to give the crude product. TFA (15 ml) was added dropwise to the mixture of the crude product in DCM (400 mL) until the solid was dissolved. Then ethyl acetate (100 mL) was dropwise to the solution until the solid separated out. The mixture was filtered. The filter cake was purified by prep-HPLC (Kromasil-C18 100×21.2 mm 5 μm Mobile Phase: ACN/H₂O (0.1% FA) Gradient: 30-40%) to give 3-[2-bromo-5-(8-chloro-4-oxo-chromen-2-yl)-4-ethoxy-phenoxy]propanoic acid (16.5 mg, 27% yield, example 39) as a white solid. ¹H NMR (400 MHz, DMSO-d₆) δ ppm 12.67 (s, 1H), 7.99 (t, J=6.8 Hz, 2H), 7.67 (s, 1H), 7.49 (t, J=7.7 Hz, 2H), 7.12 (s, 1H), 4.29 (t, J=5.9 Hz, 2H), 4.19 (q, J=6.8 Hz, 2H), 2.75 (t, J=5.9 Hz, 2H), 1.38 (t, J=6.9 Hz, 3H). MS obsd. (ESI⁺): 467.0 [(M+H)⁺], 469.0 [(M+2+H)⁺].

### Example 40

### 3-[2-Bromo-5-(8-chloro-4-oxo-chromen-2-yl)-4-(difluoromethoxy)phenoxy]propanoic acid

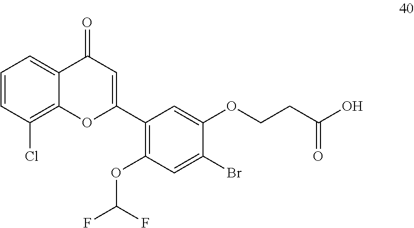 [SMILES: O=C(O)CCOc1cc(-c2cc(=O)c3cccc(Cl)c3o2)c(OC(F)F)cc1Br]

### Step 1. Synthesis of (E)-3-[4-bromo-2-(difluoromethoxy)-5-(methoxymethoxy)phenyl]-1-(3-chloro-2-hydroxy-phenyl)prop-2-en-1-one

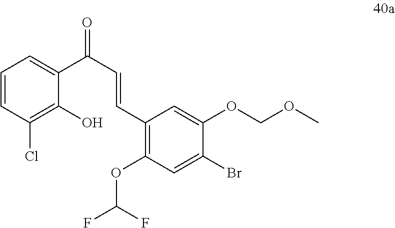 [SMILES: COCOc1cc(/C=C/C(=O)c2cccc(Cl)c2O)c(OC(F)F)cc1Br]

Compound 40a was prepared in analogy to the procedure described for the preparation of example 1 by using 4-bromo-2-(difluoromethoxy)-5-(methoxymethoxy)benzaldehyde (Intermediate AK) as the starting material instead of 4-bromo-2-methoxy-5-[(4-methoxyphenyl)methoxy]benzaldehyde (Intermediate AA) in Step 1. (E)-3-[4-Bromo-2-(difluoromethoxy)-5-(methoxymethoxy)phenyl]-1-(3-chloro-2-hydroxy-phenyl)prop-2-en-1-one (120 mg, 94% yield, compound 40a) was obtained as a yellow solid. MS obsd. (ESI⁺): 375.0 [(M+H)⁺], 377.0 [(M+2+H)⁺].

### Step 2. Synthesis of 2-[4-bromo-2-(difluoromethoxy)-5-hydroxy-phenyl]-8-chloro-chromen-4-one

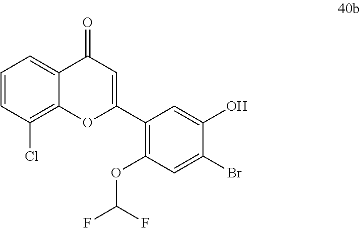 [SMILES: O=c1cc(-c2cc(O)c(Br)cc2OC(F)F)oc2c(Cl)cccc12]

Compound 40b was prepared in analogy to the procedure described for the preparation of example 1 by using (E)-3-[4-bromo-2-(difluoromethoxy)-5-(methoxymethoxy)phenyl]-1-(3-chloro-2-hydroxy-phenyl)prop-2-en-1-one (120 mg, compound 40a) as the starting material instead of (E)-3-[4-bromo-2-methoxy-5-[(4-methoxyphenyl)methoxy]phenyl]-1-(3-chloro-2-hydroxy-phenyl)prop-2-en-1-one (compound 1a) in Step 2. 2-[4-bromo-2-(difluoromethoxy)-5-hydroxy-phenyl]-8-chloro-chromen-4-one (100 mg, 85% yield, compound 40b) was obtained as a yellow solid. MS obsd. (ESI⁺): 417.0 [(M+H)⁺], 419.0 [(M+2+H)⁺].

### Step 3. Synthesis of 2-[4-bromo-2-(difluoromethoxy)-5-[2-(1,3-dioxolan-2-yl)ethoxy]phenyl]-8-chloro-chromen-4-one

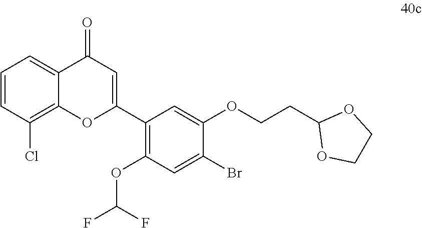 [SMILES: O=c1cc(-c2cc(OCCC3OCCO3)c(Br)cc2OC(F)F)oc2c(Cl)cccc12]

Compound 40c was prepared in analogy to the procedure described for the preparation of example 1 by using 2-[4-bromo-2-(difluoromethoxy)-5-hydroxy-phenyl]-8-chloro-chromen-4-one (100 mg, compound 40b) and 2-(2-bromoethyl)-1,3-dioxolane as the starting material instead of 2-(4-bromo-5-hydroxy-2-methoxy-phenyl)-8-chloro-chromen-4-one (compound 1c) and cis-tert-butyl 3-[2-(p-tolylsulfonyloxy)ethoxy]cyclobutanecarboxylate in Step 4. 2-[4-Bromo-2-(difluoromethoxy)-5-[2-(1,3-dioxolan-2-yl)ethoxy]phenyl]-8-chloro-chromen-4-one (80 mg, 64% yield, compound 40c) was obtained as a light yellow solid. MS obsd. (ESI⁺): 517.0 [(M+H)⁺], 519.0 [(M+2+H)⁺].

### Step 4. Synthesis of 3-[2-bromo-5-(8-chloro-4-oxo-chromen-2-yl)-4-(difluoromethoxy)phenoxy]propanal

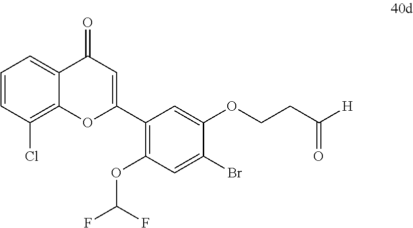 [SMILES: O=CCCOc1cc(-c2cc(=O)c3cccc(Cl)c3o2)c(OC(F)F)cc1Br]

Compound 40d was prepared in analogy to the procedure described for the preparation of example 1 by using 2-[4-bromo-2-(difluoromethoxy)-5-[2-(1,3-dioxolan-2-yl)ethoxy]phenyl]-8-chloro-chromen-4-one (80 mg, compound 40c) as the starting material instead of 2-(4-bromo-2-ethoxy-5-hydroxy-phenyl)-8-chloro-chromen-4-one (90 mg, 90% yield, compound 39b) in Step 3. 3-[2-bromo-5-(8-chloro-4-oxo-chromen-2-yl)-4-(difluoromethoxy)phenoxy]propanal (70 mg, 95% yield, compound 40d) was obtained as a yellow solid. MS obsd. (ESI⁺): 475.0 [(M+H)⁺], 476.9 [(M+2+H)⁺].

### Step 5. Synthesis of 3-[2-bromo-5-(8-chloro-4-oxo-chromen-2-yl)-4-(difluoromethoxy)phenoxy]propanoic acid

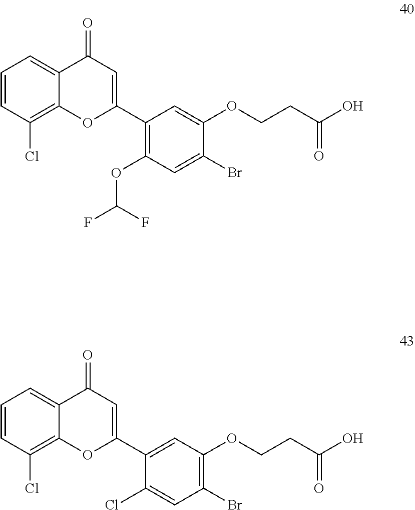 [SMILES: O=C(O)CCOc1cc(-c2cc(=O)c3cccc(Cl)c3o2)c(OC(F)F)cc1Br ; O=C(O)CCOc1cc(-c2cc(=O)c3cccc(Cl)c3o2)c(Cl)cc1Br]

Example 40 was prepared in analogy to the procedure described for the preparation of example 1 by using 3-[2-bromo-5-(8-chloro-4-oxo-chromen-2-yl)-4-(difluoromethoxy)phenoxy]propanal (70 mg, compound 40d) as the starting material instead of 3-[2-bromo-5-(8-chloro-4-oxo-chromen-2-yl)-4-ethoxy-phenoxy]propanal (60 mg, 94% yield. Compound 39d) in Step 4. 3-[2-Bromo-5-(8-chloro-4-oxo-chromen-2-yl)-4-(difluoromethoxy)phenoxy]propanoic acid (11.1 mg, 15% yield, Example 40) was obtained as a white solid. ¹H NMR (400 MHz, DMSO-d₆) δ ppm 12.49 (s, 1H), 8.04 (ddd, J=8.0, 3.7, 1.5 Hz, 2H), 7.73 (s, 1H), 7.66 (s, 1H), 7.53 (t, J=7.9 Hz, 1H), 7.30 (t, J=73.3 Hz, 1H), 6.90 (s, 1H), 4.37 (t, J=6.1 Hz, 2H), 2.78 (t, J=6.0 Hz, 2H). MS obsd. (ESI⁺): 489.0 [(M+H)⁺], 491.0 [(M+2+H)⁺].

### Example 41

### 3-[2-Bromo-5-(8-chloro-4-oxo-chromen-2-yl)-4-fluoro-phenoxy]propanoic acid

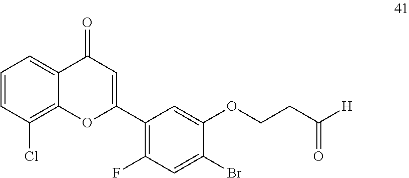 [SMILES: O=CCCOc1cc(-c2cc(=O)c3cccc(Cl)c3o2)c(F)cc1Br]

### Step 1. Synthesis of (E)-3-[4-bromo-2-fluoro-5-(methoxymethoxy)phenyl]-1-(3-chloro-2-hydroxy-phenyl)prop-2-en-1-one

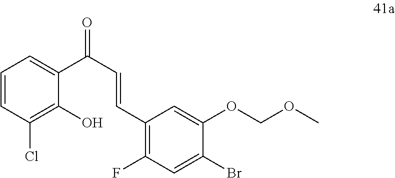 [SMILES: COCOc1cc(/C=C/C(=O)c2cccc(Cl)c2O)c(F)cc1Br]

Compound 41a was prepared in analogy to the procedure described for the preparation of example 1 by using 4-bromo-2-fluoro-5-(methoxymethoxy)benzaldehyde (Intermediate AM) as the starting material instead of 4-bromo-2-methoxy-5-[(4-methoxyphenyl)methoxy]benzaldehyde (Intermediate AA) in Step 1. (E)-3-[4-Bromo-2-fluoro-5-(methoxymethoxy)phenyl]-1-(3-chloro-2-hydroxy-phenyl)prop-2-en-1-one (350 mg, 85.2% yield, compound 41a) was obtained as a yellow solid. MS obsd. (ESI⁺): 415.0[(M+H)⁺], 416.9 [(M+H+2)⁺].

### Step 2. Synthesis of 2-(4-Bromo-2-fluoro-5-hydroxy-phenyl)-8-chloro-chromen-4-one

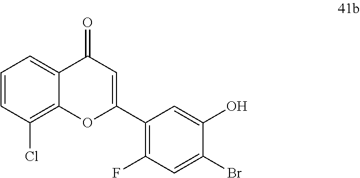 [SMILES: O=c1cc(-c2cc(O)c(Br)cc2F)oc2c(Cl)cccc12]

Compound 41b was prepared in analogy to the procedure described for the preparation of example 1 by using (E)-3-[4-bromo-2-fluoro-5-(methoxymethoxy)phenyl]-1-(3-chloro-2-hydroxy-phenyl)prop-2-en-1-one (350 mg, compound 41a) as the starting material instead of (E)-3-[4-bromo-2-methoxy-5-[(4-methoxyphenyl)methoxy]phenyl]-1-(3-chloro-2-hydroxy-phenyl)prop-2-en-1-one (compound 1a) in Step 2. 2-(4-bromo-2-fluoro-5-hydroxy-phenyl)-8-chloro-chromen-4-one (300 mg, 96.4% yield, compound 41b) was obtained as a light yellow solid. MS obsd. (ESI⁺): 368.9 [(M+H)⁺], 370.9 [(M+H+2)⁺].

### Step 3. Synthesis of 2-[4-bromo-5-[2-(1,3-dioxolan-2-yl)ethoxy]-2-fluoro-phenyl]-8-chloro-chromen-4-one

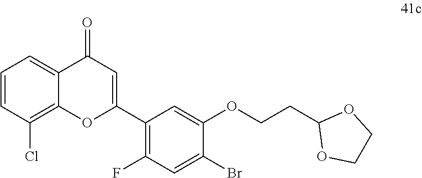 [SMILES: O=c1cc(-c2cc(OCCC3OCCO3)c(Br)cc2F)oc2c(Cl)cccc12]

Compound 41c was prepared in analogy to the procedure described for the preparation of example 1 by using 2-(4-bromo-2-fluoro-5-hydroxy-phenyl)-8-chloro-chromen-4-one (300 mg, compound 41b) and 2-(2-bromoethyl)-1,3-dioxolane as the starting material instead of 2-(4-bromo-5-hydroxy-2-methoxy-phenyl)-8-chloro-chromen-4-one (compound 1c) and cis-tert-butyl 3-[2-(p-tolylsulfonyloxy)ethoxy]cyclobutanecarboxylate in Step 4. 2-[4-Bromo-5-[2-(1,3-dioxolan-2-yl)ethoxy]-2-fluoro-phenyl]-8-chloro-chromen-4-one (60 mg, 47.21% yield, compound 41c) was obtained as a light yellow solid. MS obsd. (ESI⁺): 469.0 [(M+H)⁺], 471.0 [(M+H+2)+].

### Step 4. Synthesis of 3-[2-bromo-5-(8-chloro-4-oxo-chromen-2-yl)-4-fluoro-phenoxy]propanal

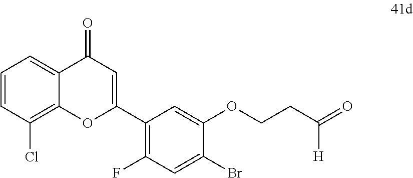 [SMILES: O=CCCOc1cc(-c2cc(=O)c3cccc(Cl)c3o2)c(F)cc1Br]

Compound 41d was prepared in analogy to the procedure described for the preparation of example 39 by using 2-[4-bromo-5-[2-(1,3-dioxolan-2-yl)ethoxy]-2-fluoro-phenyl]-8-chloro-chromen-4-one (60 mg, compound 41c) as the starting material instead of 2-(4-bromo-2-ethoxy-5-hydroxy-phenyl)-8-chloro-chromen-4-one (compound 39b) in Step 3. 3-[2-Bromo-5-(8-chloro-4-oxo-chromen-2-yl)-4-fluoro-phenoxy]propanal (60 mg, compound 41d) was obtained as a light yellow solid. MS obsd. (ESI⁺): 425.0 [(M+H)⁺], 427.0 [(M+H+2)+].

### Step 5. Synthesis of 3-[2-bromo-5-(8-chloro-4-oxo-chromen-2-yl)-4-fluoro-phenoxy]propanoic acid

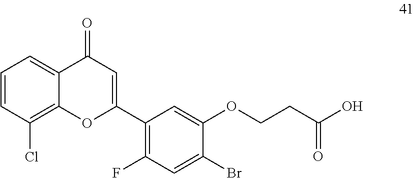 [SMILES: O=C(O)CCOc1cc(-c2cc(=O)c3cccc(Cl)c3o2)c(F)cc1Br]

Example 41 was prepared in analogy to the procedure described for the preparation of example 1 by using 3-[2-bromo-5-(8-chloro-4-oxo-chromen-2-yl)-4-fluoro-phenoxy]propanal (60 mg, compound 41d) as the starting material instead of 3-[2-bromo-5-(8-chloro-4-oxo-chromen-2-yl)-4-ethoxy-phenoxy]propanal (Compound 39d) in Step 4. 3-[2-Bromo-5-(8-chloro-4-oxo-chromen-2-yl)-4-fluoro-phenoxy]propanoic acid (26 mg, 41% yield, example 41) was obtained as a light yellow solid. MS obsd. ¹H NMR (400 MHz, DMSO-d₆) δ ppm 12.46 (s, 1H), 8.04 (t, J=7.6 Hz, 2H), 7.90 (d, J=10.5 Hz, 1H), 7.66 (d, J=6.5 Hz, 1H), 7.53 (t, J=7.9 Hz, 1H), 6.96 (s, 1H), 4.37 (t, J=5.9 Hz, 2H), 2.79 (t, J=5.9 Hz, 2H). MS obsd. (ESI⁺) [(M+H)⁺]: 441.0 [(M+H)⁺], 443.0 [(M+2+H)⁺].

### Example 42

### 3-[2-Bromo-5-(8-chloro-4-oxo-chromen-2-yl)-4-(trifluoromethoxy)phenoxy]propanoic acid

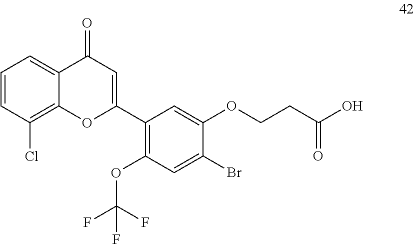 [SMILES: O=C(O)CCOc1cc(-c2cc(=O)c3cccc(Cl)c3o2)c(OC(F)(F)F)cc1Br]

### Step 1. Synthesis of 3-[2-bromo-5-[(E)-3-(3-chloro-2-hydroxy-phenyl)-3-oxo-prop-1-enyl]-4-(trifluoromethoxy)phenoxy]propanoic acid

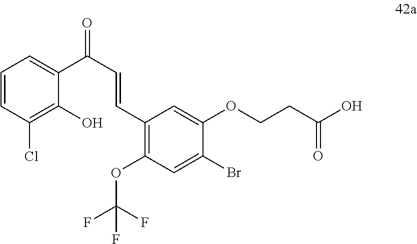 [SMILES: O=C(O)CCOc1cc(/C=C/C(=O)c2cccc(Cl)c2O)c(OC(F)(F)F)cc1Br]

Compound 42a was prepared in analogy to the procedure described for the preparation of example 1 by using 3-[2-bromo-5-formyl-4-(trifluoromethoxy)phenoxy]propanoic acid (Intermediate AN) as the starting material instead of 4-bromo-2-methoxy-5-[(4-methoxyphenyl)methoxy]benzaldehyde (Intermediate AA) in Step 1. 3-[2-Bromo-5-[(E)-3-(3-chloro-2-hydroxy-phenyl)-3-oxo-prop-1-enyl]-4-(trifluoromethoxy)phenoxy]propanoic acid (800 mg, 56% yield, compound 42a) was obtained as a light yellow solid. MS obsd. (ESI⁺): 508.9 [(M+H)⁺], 511.0 [(M+2+H)⁺].

### Step 2. Synthesis of 3-[2-bromo-5-(8-chloro-4-oxo-chromen-2-yl)-4-(trifluoromethoxy)phenoxy]propanoic acid

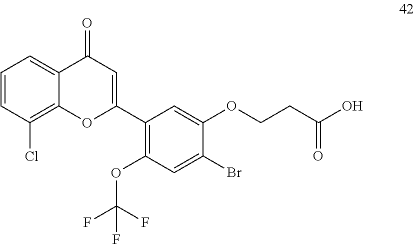 [SMILES: O=C(O)CCOc1cc(-c2cc(=O)c3cccc(Cl)c3o2)c(OC(F)(F)F)cc1Br]

Example 42 was prepared in analogy to the procedure described for the preparation of example 1 by using 3-[2-bromo-5-[(E)-3-(3-chloro-2-hydroxy-phenyl)-3-oxo-prop-1-enyl]-4-(trifluoromethoxy)phenoxy]propanoic acid (800 mg, compound 42a) as the starting material instead of (E)-3-[4-bromo-2-methoxy-5-[(4-methoxyphenyl)methoxy]phenyl]-1-(3-chloro-2-hydroxy-phenyl)prop-2-en-1-one (compound 1a) in Step 2. 3-[2-bromo-5-(8-chloro-4-oxo-chromen-2-yl)-4-(trifluoromethoxy)phenoxy]propanoic acid (58.2 mg, 29% yield, example 42) was obtained as a light yellow solid. ¹H NMR (400 MHz, DMSO-d₆) δ ppm 12.54 (s, 1H), 7.89-8.18 (m, 3H), 7.79 (s, 1H), 7.50 (s, 1H), 7.07 (s, 1H), 4.45 (t, J=5.8 Hz, 2H), 2.81 (d, J=5.7 Hz, 2H). MS obsd. (ESI⁺): 507.0 [(M+H)⁺], 509.0 [(M+2+H)⁺].

### Example 43

### 3-[2-Bromo-4-chloro-5-(8-chloro-4-oxo-chromen-2-yl)phenoxy]propanoic acid

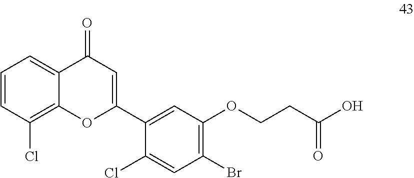 [SMILES: O=C(O)CCOc1cc(-c2cc(=O)c3cccc(Cl)c3o2)c(Cl)cc1Br]

### Step 1. Synthesis of (E)-3-[4-bromo-2-chloro-5-(methoxymethoxy)phenyl]-1-(3-chloro-2-hydroxy-phenyl)prop-2-en-1-one

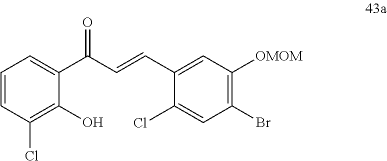

Compound 43a was prepared in analogy to the procedure described for the preparation of example 1 by using 4-bromo-2-chloro-5-(methoxymethoxy)benzaldehyde (Intermediate AL) as the starting material instead of 4-bromo-2-methoxy-5-[(4-methoxyphenyl)methoxy]benzaldehyde (Intermediate AA) in Step 1. (E)-3-[4-Bromo-2-chloro-5-(methoxymethoxy)phenyl]-1-(3-chloro-2-hydroxy-phenyl)prop-2-en-1-one (327 mg, 70% yield, compound 43a) was obtained as a light yellow solid. MS obsd. (ESI⁺): 430.9 [(M+H)⁺], 432.9 [(M+2+H)⁺].

### Step 2. Synthesis of 2-[4-bromo-2-chloro-5-(methoxymethoxy)phenyl]-8-chloro-chromen-4-one

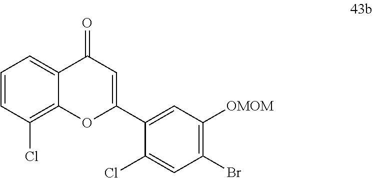

Compound 43b was prepared in analogy to the procedure described for the preparation of example 1 by using (E)-3-[4-bromo-2-chloro-5-(methoxymethoxy)phenyl]-1-(3-chloro-2-hydroxy-phenyl)prop-2-en-1-one (327 mg, compound 43a) as the starting material instead of (E)-3-[4-bromo-2-methoxy-5-[(4-methoxyphenyl)methoxy]phenyl]-1-(3-chloro-2-hydroxy-phenyl)prop-2-en-1-one (compound 1a) in Step 2. 2-[4-bromo-2-chloro-5-(methoxymethoxy)phenyl]-8-chloro-chromen-4-one (288 mg, 96% yield, compound 43b) was obtained as a light yellow solid. MS obsd. (ESI⁺): 428.9 [(M+H)⁺], 430.9 [(M+2+H)⁺], 432.9 [(M+4+H)⁺].

### Step 3. Synthesis of 2-(4-bromo-2-chloro-5-hydroxy-phenyl)-8-chloro-chromen-4-one

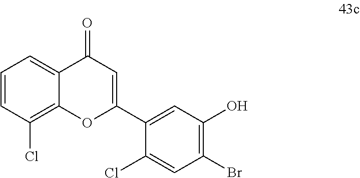 [SMILES: O=c1cc(-c2cc(O)c(Br)cc2Cl)oc2c(Cl)cccc12]

To a solution of 2-[4-bromo-2-chloro-5-(methoxymethoxy)phenyl]-8-chloro-chromen-4-one (288.0 mg) in DCM (3 mL) was added trifluoroacetic acid (2.0 mL) and stirred at room temperature for 2 hrs. The reaction mixture was concentrated in vacuo to give 2-(4-bromo-2-chloro-5-hydroxy-phenyl)-8-chloro-chromen-4-one (240 mg, 92% yield, compound 43c) was obtained as a light yellow solid. MS obsd. (ESI⁺): 384.9 [(M+H)⁺], 386.9 [(M+2+H)⁺], 388.9 [(M+4+H)⁺]

### Step 4. Synthesis of 8-chloro-2-[2-[2-(1,3-dioxolan-2-yl)ethoxy]-4-(trifluoromethyl)phenyl]-7-(1-methylpyrazol-3-yl)chromen-4-one

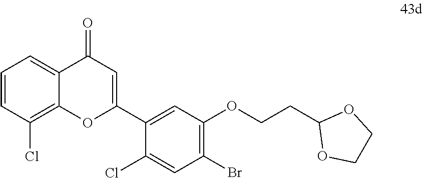 [SMILES: O=c1cc(-c2cc(OCCC3OCCO3)c(Br)cc2Cl)oc2c(Cl)cccc12]

Compound 43d was prepared in analogy to the procedure described for the preparation of example 1 by using 2-(4-bromo-2-chloro-5-hydroxy-phenyl)-8-chloro-chromen-4-one (compound 43c) and 2-(2-bromoethyl)-1,3-dioxolane as the starting material instead of 2-(4-bromo-5-hydroxy-2-methoxy-phenyl)-8-chloro-chromen-4-one (compound 1c) and tert-butyl 3-[2-(p-tolylsulfonyloxy)ethoxy]cyclobutanecarboxylate in Step 4. 8-Chloro-2-[2-[2-(1,3-dioxolan-2-yl)ethoxy]-4-(trifluoromethyl)phenyl]-7-(1-methylpyrazol-3-yl)chromen-4-one (160 mg, 81% yield, compound 43d) was obtained as a light yellow solid. MS obsd. (ESI⁺): 485.0 [(M+H)⁺], 487.0 [(M+2+H)⁺], 489.0 [(M+4+H)⁺]

### Step 5. Synthesis of 3-[2-bromo-4-chloro-5-(8-chloro-4-oxo-chromen-2-yl)phenoxy]propanal

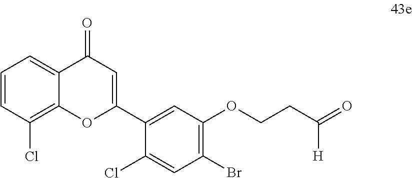 [SMILES: O=CCCOc1cc(-c2cc(=O)c3cccc(Cl)c3o2)c(Cl)cc1Br]

Compound 43e was prepared in analogy to the procedure described for the preparation of example 1 by using 8-chloro-2-[2-[2-(1,3-dioxolan-2-yl)ethoxy]-4-(trifluoromethyl)phenyl]-7-(1-methylpyrazol-3-yl)chromen-4-one (160 mg, compound 43d) as the starting material instead of 2-(4-bromo-2-ethoxy-5-hydroxy-phenyl)-8-chloro-chromen-4-one (90 mg, 90% yield, compound 39b) in Step 3. 3-[2-bromo-4-chloro-5-(8-chloro-4-oxo-chromen-2-yl)phenoxy]propanal (198 mg, compound 43e) was obtained as a crude solid. MS obsd. (ESI⁺): 440.9 [(M+H)⁺], 443.0 [(M+2+H)⁺], 445.0 [(M+4+H)⁺].

### Step 6. Synthesis of 3-[2-bromo-4-chloro-5-(8-chloro-4-oxo-chromen-2-yl)phenoxy]propanoic acid

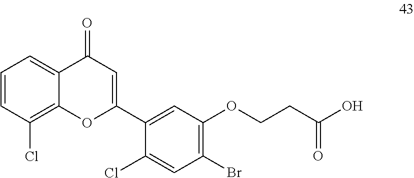 [SMILES: O=C(O)CCOc1cc(-c2cc(=O)c3cccc(Cl)c3o2)c(Cl)cc1Br]

Example 43 was prepared in analogy to the procedure described for the preparation of example 1 by using 3-[2-bromo-4-chloro-5-(8-chloro-4-oxo-chromen-2-yl)phenoxy]propanal (198 mg, compound 43e) as the starting material instead of 3-[2-bromo-5-(8-chloro-4-oxo-chromen-2-yl)-4-ethoxy-phenoxy]propanal (Compound 39d) in Step 4. 3-[2-Bromo-4-chloro-5-(8-chloro-4-oxo-chromen-2-yl)phenoxy]propanoic acid (48.9 mg, Example 43) was obtained as a white solid. ¹H NMR (400 MHz, DMSO-d₆) δ ppm 8.07 (t, J=8.3 Hz, 2H), 7.69 (d, J=9.0 Hz, 1H), 7.56 (d, J=7.9 Hz, 1H), 7.42 (d, J=9.1 Hz, 1H), 6.71 (s, 1H), 4.33 (t, J=5.9 Hz, 2H), 2.74 (t, J=5.9 Hz, 2H). MS obsd. (ESI⁺): 456.9 [(M+H)⁺], 458.9 [(M+2+H)⁺], 460.9. [(M+4+H)⁺].

### Example 44

### 2-[3-[6-Bromo-3-(8-chloro-4-oxo-chromen-2-yl)-2-methoxy-phenoxy]propylamino]-2-oxo-acetic acid

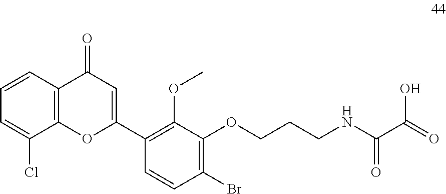 [SMILES: COc1c(-c2cc(=O)c3cccc(Cl)c3o2)ccc(Br)c1OCCCNC(=O)C(=O)O]

### Step 1. Synthesis of tert-butyl N-[3-[6-bromo-3-(8-chloro-4-oxo-chromen-2-yl)-2-methoxy-phenoxy]propyl]carbamate

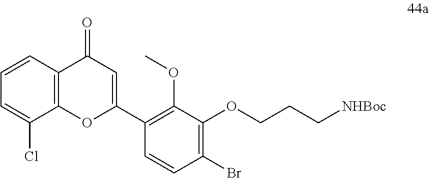 [SMILES: COc1c(-c2cc(=O)c3cccc(Cl)c3o2)ccc(Br)c1OCCCNC(=O)OC(C)(C)C]

To a solution of 2-(4-bromo-3-hydroxy-2-methoxy-phenyl)-8-chloro-chromen-4-one (200.0 mg, 0.520 mmol, compound 34b) and potassium iodide (174.0 mg) in dry DMF (20 mL) was added potassium tert-butoxide (117.62 mg). After addition, the reaction mixture was stirred at 25° C. for 0.5 h. Then 3-(BOC-amino)propyl bromide (250 mg) was added. After addition, the reaction was stirred at 25° C. for 16 hrs. Then it was cooled to 25° C., potassium tert-butoxide (117.62 mg) was added. After addition, the reaction was stirred at 25° C. for 0.5 h. Then 3-(BOC-amino)propyl bromide (250 mg) was added. After addition, the reaction was stirred at 25° C. for 16 hrs. Then it was poured into H₂O (50 mL), extracted with EtOAc (20 mL) three times. The combined organics were washed with brine (100 mL) three times, dried over anhydrous Na₂SO₄, filtered and concentrated in vacuo. The resulting oil was purified by flash silica gel column chromatography (eluting with EtOAc/PE (V/V)=3/7) to give tert-butyl N-[3-[6-bromo-3-(8-chloro-4-oxo-chromen-2-yl)-2-methoxy-phenoxy]propyl]carbamate (90 mg, 31.87% yield, compound 44a) as a yellow solid. MS obsd. (ESI⁺): 560.0 [(M+Na)⁺], 562.0 [(M+2+Na)⁺].

### Step 2. Synthesis of 2-[3-(3-aminopropoxy)-4-bromo-2-methoxy-phenyl]-8-chloro-chromen-4-one

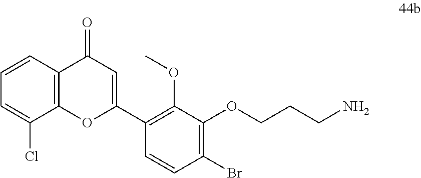 [SMILES: COc1c(-c2cc(=O)c3cccc(Cl)c3o2)ccc(Br)c1OCCCN]

To a solution of tert-butyl N-[3-[6-bromo-3-(8-chloro-4-oxo-chromen-2-yl)-2-methoxy-phenoxy]propyl]carbamate (90.0 mg, 0.170 mmol) in DCM (10 mL) at 25° C. was added trifluoroacetic acid (1.0 mL, 13 mmol) dropwise. After addition, this mixture was stirred at 25° C. for 3 hrs. Then the reaction mixture was concentrated in vacuo to give 2-[3-(3-aminopropoxy)-4-bromo-2-methoxy-phenyl]-8-chloro-chromen-4-one (72 mg, compound 44b) as a yellow solid. MS obsd. (ESI⁺): 438.0 [(M+H)⁺], 440.0 [(M+2+H)⁺].

### Step 3. Synthesis of ethyl 2-[3-[6-bromo-3-(8-chloro-4-oxo-chromen-2-yl)-2-methoxy-phenoxy]propylamino]-2-oxo-acetate

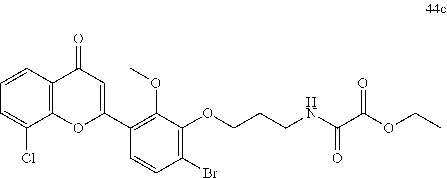 [SMILES: CCOC(=O)C(=O)NCCCOc1c(Br)ccc(-c2cc(=O)c3cccc(Cl)c3o2)c1OC]

To a solution of 2-[3-(3-aminopropoxy)-4-bromo-2-methoxy-phenyl]-8-chloro-chromen-4-one (72.0 mg), N,N-diisopropylethylamine (0.11 mL) in DCM (10 mL) at 0° C. was added ethyl oxalyl chloride (33.61 mg) dropwise. After addition, this mixture was stirred at 25° C. for 6 hrs. Then it was concentrated in vacuo. The crude product was purified by reverse silica gel chromatography (eluting with ACN/water from 0% to 50%, 0.1% FA in water) to give ethyl 2-[3-[6-bromo-3-(8-chloro-4-oxo-chromen-2-yl)-2-methoxy-phenoxy]propylamino]-2-oxo-acetate (40 mg, compound 44c) as a white solid. MS obsd. (ESI⁺): 560.0 [(M+H)⁺], 562.0 [(M+2+H)⁺].

### Step 4. Synthesis of 2-[3-[6-bromo-3-(8-chloro-4-oxo-chromen-2-yl)-2-methoxy-phenoxy]propylamino]-2-oxo-acetic acid

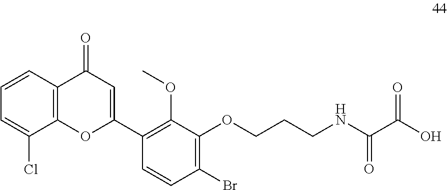 [SMILES: COc1c(-c2cc(=O)c3cccc(Cl)c3o2)ccc(Br)c1OCCCNC(=O)C(=O)O]

To a solution of ethyl 2-[3-[6-bromo-3-(8-chloro-4-oxo-chromen-2-yl)-2-methoxy-phenoxy]propylamino]-2-oxo-acetate (40.0 mg) in THF (10 mL) at 0° C. was added lithium hydroxide (0.74 mL). After being stirred at 25° C. for 16 hrs, the reaction mixture was cooled to 0° C., acidified to pH=3 with 1 M HCl at 0° C. and concentrated in vacuo. The residue was purified by prep-HPLC (eluting with ACN in water 0% to 20%, 0.1% NH₄OH in water) to afford 2-[3-[6-bromo-3-(8-chloro-4-oxo-chromen-2-yl)-2-methoxy-phenoxy]propylamino]-2-oxo-acetic acid (15 mg, example 44) as an off-white solid. ¹H NMR (400 MHz, DMSO-d₆) δ ppm 13.85 (s, 1H), 8.94 (s, 1H), 8.03 (d, J=7.8 Hz, 2H), 7.66 (d, J=8.6 Hz, 1H), 7.59 (d, J=8.9 Hz, 1H), 7.53 (t, J=7.5 Hz, 1H), 6.93 (s, 1H), 4.05 (s, 1H), 3.92 (s, 3H), 3.39 (s, 2H), 2.01 (s, 2H). MS obsd. (ESI⁺): 510.0 [(M+H)⁺], 512.0 [(M+2+H)⁺].

### Example 45

### 2-[2-[2-Bromo-5-(8-chloro-4-oxo-chromen-2-yl)-4-methoxy-phenoxy]ethylamino]-2-oxo-acetic acid

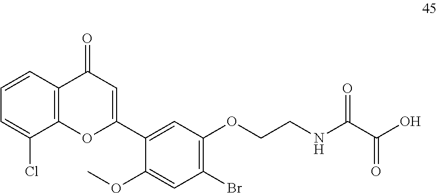 [SMILES: COc1cc(Br)c(OCCNC(=O)C(=O)O)cc1-c1cc(=O)c2cccc(Cl)c2o1]

### Step 1. Synthesis of tert-butyl N-[2-[2-bromo-5-(8-chloro-4-oxo-chromen-2-yl)-4-methoxy-phenoxy]ethyl]carbamate

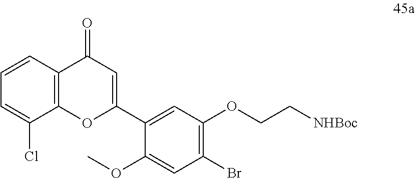 [SMILES: COc1cc(Br)c(OCCNC(=O)OC(C)(C)C)cc1-c1cc(=O)c2cccc(Cl)c2o1]

Compound 45a was prepared in analogy to the procedure described for the preparation of example 44 by using 2-(4-bromo-5-hydroxy-2-methoxy-phenyl)-8-chloro-chromen-4-one (compound 1c) and tert-butyl N-(2-bromoethyl)carbamate as the starting material instead of 2-(4-bromo-3-hydroxy-2-methoxy-phenyl)-8-chloro-chromen-4-one (compound 34b) and 3-(BOC-amino)propyl bromide in Step 1. Tert-Butyl N-[2-[2-bromo-5-(8-chloro-4-oxo-chromen-2-yl)-4-methoxy-phenoxy]ethyl]carbamate (240 mg, compound 45a) was obtained as a yellow solid. MS obsd. (ESI⁺): 524.1 [(M+H)⁺], 526.1 [(M+2+H)⁺].

### Step 2. Synthesis of 2-[5-(2-aminoethoxy)-4-bromo-2-hydroxy-phenyl]-8-chloro-chroman-4-one

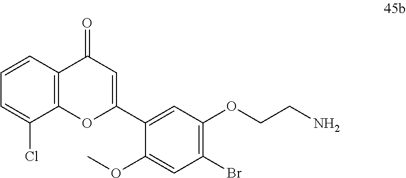 [SMILES: COc1cc(Br)c(OCCN)cc1-c1cc(=O)c2cccc(Cl)c2o1]

Compound 45b was prepared in analogy to the procedure described for the preparation of example 44 by using tert-butyl N-[2-[2-bromo-5-(8-chloro-4-oxo-chromen-2-yl)-4-methoxy-phenoxy]ethyl]carbamate (240 mg, compound 45a) as the starting material instead of tert-butyl N-[3-[6-bromo-3-(8-chloro-4-oxo-chromen-2-yl)-2-methoxy-phenoxy]propyl]carbamate (compound 44a) in Step 2. 2-[5-(2-Aminoethoxy)-4-bromo-2-hydroxy-phenyl]-8-chloro-chroman-4-one (198 mg, compound 45b) was obtained as a yellow solid. MS obsd. (ESI⁺): 424.0 [(M+H)⁺], 426.1 [(M+2+H)⁺].

### Step 3. Synthesis of ethyl 2-[2-[2-bromo-5-(8-chloro-4-oxo-chromen-2-yl)-4-methoxy-phenoxy]ethylamino]-2-oxo-acetate

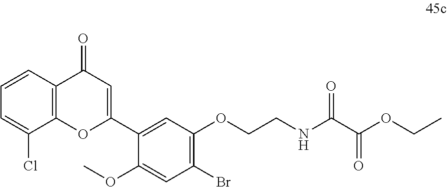 [SMILES: CCOC(=O)C(=O)NCCOc1cc(-c2cc(=O)c3cccc(Cl)c3o2)c(OC)cc1Br]

Compound 46c was prepared in analogy to the procedure described for the preparation of example 44 by using 2-[5-(2-aminoethoxy)-4-bromo-2-hydroxy-phenyl]-8-chloro-chroman-4-one (198 mg, compound 45b) as the starting material instead of 2-[3-(3-aminopropoxy)-4-bromo-2-methoxy-phenyl]-8-chloro-chromen-4- (72 mg, compound 44b) in Step 3. Ethyl 2-[2-[2-bromo-5-(8-chloro-4-oxo-chromen-2-yl)-4-methoxy-phenoxy]ethylamino]-2-oxo-acetate (100 mg, compound 45c) was obtained as a yellow solid. MS obsd. (ESI⁺): 524.0 [(M+H)⁺], 526.0 [(M+2+H)⁺].

### Step 4. Synthesis of 2-[2-[2-bromo-5-(8-chloro-4-oxo-chromen-2-yl)-4-methoxy-phenoxy]ethylamino]-2-oxo-acetic acid

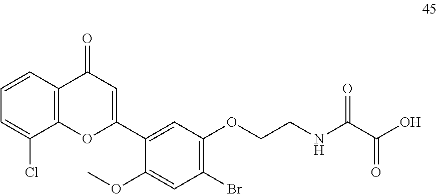 [SMILES: COc1cc(Br)c(OCCNC(=O)C(=O)O)cc1-c1cc(=O)c2cccc(Cl)c2o1]

Example 45 was prepared in analogy to the procedure described for the preparation of example 44 by using ethyl 2-[2-[2-bromo-5-(8-chloro-4-oxo-chromen-2-yl)-4-methoxy-phenoxy]ethylamino]-2-oxo-acetate (100 mg, compound 45c) as the starting material instead of ethyl 2-[3-[6-bromo-3-(8-chloro-4-oxo-chromen-2-yl)-2-methoxy-phenoxy]propylamino]-2-oxo-acetate (40 mg, compound 44c) in Step 4. 2-[2-[2-Bromo-5-(8-chloro-4-oxo-chromen-2-yl)-4-methoxy-phenoxy]ethylamino]-2-oxo-acetic acid (30 mg, example 45) was obtained as a yellow solid. ¹H NMR (400 MHz, DMSO-d₆) δ ppm 8.90 (t, J=5.5 Hz, 1H), 8.00 (t, J=6.9 Hz, 2H), 7.66 (s, 1H), 7.42-7.51 (m, 2H), 7.06 (s, 1H), 4.20 (t, J=5.7 Hz, 2H), 3.92 (s, 3H), 3.58 (dd, J=11.8, 6.0 Hz, 2H). MS obsd. (ESI⁺): 496.0 [(M+H)⁺], 498.0 [(M+2+H)⁺].

### Example 46

### 2-[3-[2-Bromo-5-(8-chloro-4-oxo-chromen-2-yl)-4-methoxy-phenoxy]propylamino]-2-oxo-acetic acid

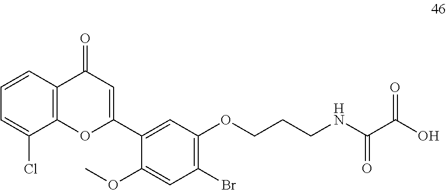 [SMILES: COc1cc(Br)c(OCCCNC(=O)C(=O)O)cc1-c1cc(=O)c2cccc(Cl)c2o1]

### Step 1. Synthesis of tert-butyl N-[3-[2-bromo-5-(8-chloro-4-oxo-chromen-2-yl)-4-methoxy-phenoxy]propyl]carbamate

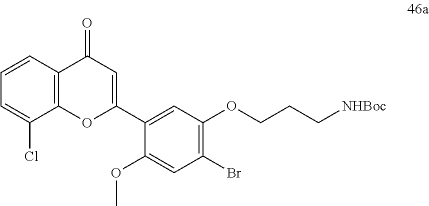 [SMILES: COc1cc(Br)c(OCCCNC(=O)OC(C)(C)C)cc1-c1cc(=O)c2cccc(Cl)c2o1]

Compound 46a was prepared in analogy to the procedure described for the preparation of example 44 by using 2-(4-bromo-5-hydroxy-2-methoxy-phenyl)-8-chloro-chromen-4-one (compound 1c) as the starting material instead of 2-(4-bromo-3-hydroxy-2-methoxy-phenyl)-8-chloro-chromen-4-one (compound 34b) in Step 1. tert-Butyl N-[3-[2-bromo-5-(8-chloro-4-oxo-chromen-2-yl)-4-methoxy-phenoxy]propyl]carbamate (170 mg, 60.2% yield, compound 45a) was obtained as a yellow solid.

### Step 2. Synthesis of 2-[5-(3-aminopropoxy)-4-bromo-2-hydroxy-phenyl]-8-chloro-chroman-4-one

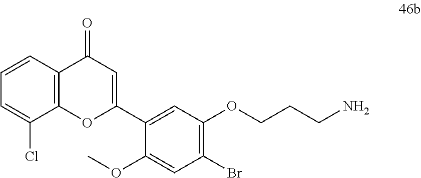 [SMILES: COc1cc(Br)c(OCCCN)cc1-c1cc(=O)c2cccc(Cl)c2o1]

Compound 46b was prepared in analogy to the procedure described for the preparation of example 44 by using tert-Butyl N-[3-[2-bromo-5-(8-chloro-4-oxo-chromen-2-yl)-4-methoxy-phenoxy]propyl]carbamate (compound 45a) as the starting material instead of tert-butyl N-[3-[6-bromo-3-(8-chloro-4-oxo-chromen-2-yl)-2-methoxy-phenoxy]propyl]carbamate (compound 44a) in Step 2. 2-[5-(3-aminopropoxy)-4-bromo-2-hydroxy-phenyl]-8-chloro-chroman-4-one (134 mg, compound 46b) was obtained as a yellow solid. MS obsd. (ESI⁺): 438.0 [(M+H)⁺], 440.0 [(M+2+H)⁺].

### Step 3. Synthesis of ethyl 2-[3-[2-bromo-5-(8-chloro-4-oxo-chromen-2-yl)-4-methoxy-phenoxy]propylamino]-2-oxo-acetate

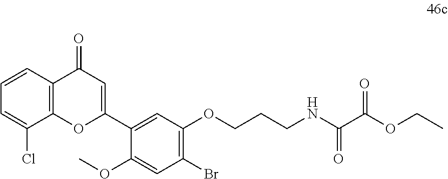 [SMILES: CCOC(=O)C(=O)NCCCOc1cc(-c2cc(=O)c3cccc(Cl)c3o2)c(OC)cc1Br]

Compound 46c was prepared in analogy to the procedure described for the preparation of example 44 by using 2-[5-(3-aminopropoxy)-4-bromo-2-hydroxy-phenyl]-8-chloro-chroman-4-one (134 mg, compound 46b) as the starting material instead of 2-[3-(3-aminopropoxy)-4-bromo-2-methoxy-phenyl]-8-chloro-chromen-4- (compound 44b) in Step 3. Ethyl 2-[3-[2-bromo-5-(8-chloro-4-oxo-chromen-2-yl)-4-methoxy-phenoxy]propylamino]-2-oxo-acetate (130 mg, 69% yield, compound 46c) was obtained as a yellow solid. MS obsd. (ESI⁺): 538.1 [(M+H)⁺], 540.1 [(M+2+H)⁺].

### Step 4. Synthesis of 2-[3-[2-bromo-5-(8-chloro-4-oxo-chromen-2-yl)-4-methoxy-phenoxy]propylamino]-2-oxo-acetic acid

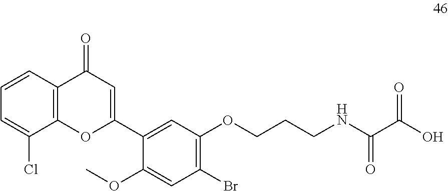 [SMILES: COc1cc(Br)c(OCCCNC(=O)C(=O)O)cc1-c1cc(=O)c2cccc(Cl)c2o1]

Example 46 was prepared in analogy to the procedure described for the preparation of example 44 by using ethyl 2-[3-[2-bromo-5-(8-chloro-4-oxo-chromen-2-yl)-4-methoxy-phenoxy]propylamino]-2-oxo-acetate (compound 46c) as the starting material instead of ethyl 2-[3-[6-bromo-3-(8-chloro-4-oxo-chromen-2-yl)-2-methoxy-phenoxy]propylamino]-2-oxo-acetate (40 mg, compound 44c) in Step 4. 2-[3-[2-Bromo-5-(8-chloro-4-oxo-chromen-2-yl)-4-methoxy-phenoxy]propylamino]-2-oxo-acetic acid (25 mg, 68% yield, example 46) was obtained as a yellow solid. ¹H NMR (400 MHz, DMSO-d₆) δ ppm 13.73 (s, 1H), 8.85 (s, 1H), 8.00 (t, J=6.8 Hz, 2H), 7.64-7.68 (m, 1H), 7.54 (s, 0.83H), 7.51 (t, J=7.8 Hz, 1H), 7.42 (s, 0.18H), 7.07 (s, 1H), 4.11 (t, J=5.7 Hz, 2H), 3.94 (s, 3H), 3.33-3.36 (m, 2H), 1.98 (t, J=5.7 Hz, 2H). MS obsd. (ESI⁺): 510.0 [(M+H)⁺], 512.0 [(M+2+H)⁺].

### BIOLOGICAL EXAMPLES

### BIO-Example 1: Engineered HepDES19 Primary Screen Assay

The assay was employed to screen for novel cccDNA inhibitors. HepDES19 is a cccDNA-producing cell line. In this cell line, HBeAg in the cell culture supernatant as surrogate marker, as HBeAg production depends on cccDNA level and activity. HepDES19 is an engineered cell line which contains a 1.1 unit length HBV genome, and pgRNA transcription from the transgene is controlled by Tetracycline (Tet). In the absence of Tet, pgRNA transcription will be induced, but HBV e antigen (HBeAg) could not be produced from this pgRNA due to very short leader sequence before the HBeAg start codon and the start codon is disrupted. Only after cccDNA is formed, the missing leader sequence and start codon mutation would be restored from the 3′-terminal redundancy of pgRNA, and then HBeAg could be synthesized. Therefore, HBeAg could be used as a surrogate marker for cccDNA (Zhou, T. et al., Antiviral Res. (2006), 72(2), 116-124; Guo, H. et al., J. Virol. (2007), 81(22), 12472-12484).

HepDES19 cells were seeded at 2×10⁶ cells per T150 flask and cultured with the culture medium (Dulbecco's Modified Eagle Medium: Nutrient Mixture F-12 [DMEM-F12, Gibco Cat. 11320-82], 10% Fetal Bovine Serum [FBS, Clontech Cat. 631101], 0.1 mM Non-Essential Amino Acids Solution [NEAA, Gibco Cat. 11140-050], 50 μg/mL Penicillin-Streptomycin [PS, Invitrogen Cat. 15140-163], 500 μg/mL Geneticin [G418, Invitrogen Cat. 10131-027]) containing 3 μg/mL Tet (Sigma, Cat. 87128) for 5 days. Cells were then seeded at 4×10⁶ cells per T150 in the same culture medium as described above in the absence of Tet for 8 days. Cells were then harvested and frozen at density of 2×10⁶ cells per ML. For compound testing, the frozen cells were thawed and seeded into 96-well plates at a density of 6×10⁴ cells per well. At 24 hours after seeding, half log serial dilutions of compounds made with Dimethyl sulfoxide (DMSO, Sigma, Cat. D2650) were further diluted with the same culture medium as described above before they were added to the cells to reach desired final compound concentrations and 1% DMSO concentration. Plates were then incubated at 37° C. for another 5 days before measurement of HBeAg level and cell viability. Intracellular HBeAg level were measured with enzyme-linked immunosorbent assay (ELISA) kit (Shanghai Kehua Diagnostic Medical Products Co., Ltd). Cell viability was assessed using Cell Counting Kit-8 (DonJindo, Cat. CK04-20). IC₅₀ values were derived from the dose-response curve using 4 parameter logistic curve fit method.

The compounds of the present invention were tested for their capacity to inhibit extracellular HBeAg level as described herein. The compounds of this invention were found to have IC₅₀ below 50 μM. Particular compounds of formula (I) were found to have IC₅₀ below 5.0 μM. Results of HepDES19 primary screen assay are given in Table 1.

TABLE 1
Activity data in HepDES19 primary screen assay

| Example No. | IC₅₀ (μM) |
|---|---|
| 1 | 3.18 |
| 2 | 1.19 |
| 3 | 0.58 |
| 4 | 1.96 |
| 5 | 6.08 |
| 6 | 1.66 |
| 7 | 4.51 |
| 8 | 1.50 |
| 9 | 5.20 |
| 10 | 1.54 |
| 11 | 2.43 |
| 12 | 0.62 |
| 13 | 0.19 |
| 14 | 6.32 |
| 15 | 0.49 |
| 16 | 6.36 |
| 17 | 0.72 |
| 18 | 0.52 |
| 19 | 7.93 |
| 20 | 2.14 |
| 21 | 4.12 |
| 22 | 0.32 |
| 23 | 7.36 |
| 24 | 1.46 |
| 25 | 7.51 |
| 26 | 1.07 |
| 27 | 3.24 |
| 28 | 0.56 |
| 29 | 3.24 |
| 30 | 7.51 |
| 31 | 4.66 |
| 32 | 7.13 |
| 33 | 6.75 |
| 34 | 0.94 |
| 35 | 0.19 |
| 36 | 3.13 |
| 37 | 10.1 |
| 38 | 1.13 |
| 39 | 0.11 |
| 40 | 1.39 |
| 41 | 0.133 |
| 42 | 2.63 |
| 43 | 11.7 |
| 44 | 1.44 |
| 45 | 1.40 |
| 46 | 0.61 |

### BIO-Example 2: Cryopreserved Primary Human Hepatocytes (PHH) Assay

This assay is used to confirm the anti-HBV effect of the compounds in HBV PHH infection assay. Cryopreserved PHH (BioreclamationIVT, Lot YJM) was thawed at 37° C. and gently transferred into pre-warmed InVitroGRO HT medium (BioreclamationIVT, Cat. S03317). The mixture was centrifuged at 70 relative centrifugal force (RCF) for 3 minutes at RT, and the supernatant was discarded. Pre-warmed InVitroGRO CP medium (BioreclamationIVT, Cat #S03316) was added to the cell pellet to gently re-suspend cells. The cells were seeded at the density of 5.8×10⁴ cells per well to collagen I coated 96-well plate (Gibco, Cat. A1142803) with the InVitroGRO CP medium. All plates were incubated at 37° C. with 5% CO₂ and 85% humidity.

At 20 hours after plating, the medium was changed to PHH culture medium (Dulbecco's Modified Eagle Medium (DMEM)/F12 (1:1) (Gibco, Cat. 11320-033), 10% fetal bovine serum (Gibco Cat. 10099141), 100 U/mL penicillin, 100 μg/mL streptomycin (Gibco, Cat. 151401-122), ng/mL human epidermal growth factor (Invitrogen Cat. PHG0311L), 20 ng/mL dexamethasone (Sigma, Cat. D4902) and 250 ng/mL human recombinant insulin (Gibco, Cat. 12585-014)). And the cells were incubated at 37° C. with 5% CO₂ and 85% humidity for 4 hours. The medium was then changed to pre-warmed PHH culture medium containing 4% polyethylene glycol (PEG) MW8000 (Sigma, Cat. P1458-50ML) and 1% DMSO (Sigma, Cat. D2650). 5.8×10⁶ genomic equivalents of HBV were added into the medium.

At 24 hours post-infection, the cells were gently washed with PBS and refreshed with PHH culture medium supplemented with 1% DMSO, and 0.25 mg/mL Matrix gel (Corning, Cat. 356237) at 200 μL per well. All plates were immediately placed in at 37° C. CO₂ incubator.

24 hours later, serial dilutions of compounds made with DMSO were further diluted with the same culture medium (PHH culture medium supplemented with 1% DMSO and 0.25 mg/mL Matrix gel as described above) before they were added to the cells to reach desired final compound concentrations and 1% DMSO concentration. The medium containing the compounds were refreshed every three days.

At 9 days post-compound treatment, extracellular HBsAg level were measured with Chemiluminescence Immuno Assay (CLIA) kit (Autobio, HBsAg Quantitative CLIA). Extracellular HBV DNA was extracted by MagNA Pure 96 system (Roche) and then determined by quantitative PCR with the following primers and probe:

HBV-Forward Primer (SEQ ID NO: 1):
AAGAAAAACCCCGCCTGTAA (5′ to 3′);
HBV-Reverse Primer (SEQ ID NO: 2):
CCTGTTCTGACTACTGCCTCTCC (5′ to 3′);
HBV-Probe:
5′ + tetramethylrhodamine + SEQ ID NO: 3 + black
hole quencher 2-3′, wherein SEQ ID NO: 3 is
CCTGATGTGATGTTCTCCATGTTCAGC.

HBsAg IC₅₀ and HBV DNA IC₅₀ values were derived from the dose-response curve using 4 parameter logistic curve fit method. The compounds of formula (I) have HBsAg IC₅₀<20 μM, particularly <1 μM; and HBV DNA IC₅₀<50 μM. Results of Cryopreserved PHH assay are given in Table D2.

TABLE D2
HBsAg IC₅₀ data in Cryopreserved PHH assay

| Example No. | HBsAg IC₅₀ (μM) |
|---|---|
| 1 | 8.02 |
| 2 | 3.85 |
| 5 | 4.09 |
| 8 | 5.13 |
| 11 | 7.40 |
| 15 | 1.96 |
| 22 | 6.59 |
| 31 | 10.2 |
| 35 | 3.91 |
| 36 | 9.26 |

1. A compound of formula (I), 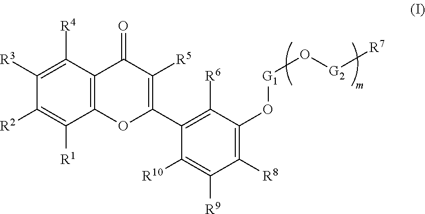
wherein:
R¹ is halogen;
R² is selected from H, OH, halogen, C₁₋₆alkyl, haloC₁₋₆alkyl and C₁₋₆alkoxy;
R³ is selected from H, OH, halogen, C₁₋₆alkyl, haloC₁₋₆alkyl and C₁₋₆alkoxy;
R⁴ is selected from H, OH, halogen, C₁₋₆alkyl, haloC₁₋₆alkyl and C₁₋₆alkoxy;
R⁵ is selected from H, OH, halogen, C₁₋₆alkyl, haloC₁₋₆alkyl and C₁₋₆alkoxy;
R⁶ is selected from H, OH, halogen, C₁₋₆alkyl, haloC₁₋₆alkyl and C₁₋₆alkoxy;
R⁷ is selected from carboxy, C₁₋₆alkoxycarbonyl, carboxycarbonylamino, C₃₋₇cycloalkylsulfonylaminocarbonyl and C₁₋₆alkoxycarbonylcarbonylamino;
R⁸ is selected from H, OH, halogen, C₁₋₆alkyl, haloC₁₋₆alkyl and C₁₋₆alkoxy;
R⁹ is selected from H, OH, halogen, C₁₋₆alkyl, haloC₁₋₆alkyl and C₁₋₆alkoxy;
R¹⁰ is selected from H, OH, halogen, C₁₋₆alkyl, haloC₁₋₆alkyl, C₁₋₆alkoxy and haloC₁₋₆alkoxy;
G₁ is selected from C₁₋₆alkyl and C₃₋₇cycloalkyl;
G₂ is selected from C₁₋₆alkyl and C₃₋₇cycloalkyl; and
m is 0 or 1;
or a pharmaceutically acceptable salt thereof.

2. A compound according to claim 1, wherein
R² is H;
R³ is H;
R⁴ is H;
R⁵ is H;
R⁶ is selected from H and C₁₋₆alkoxy;
R⁷ is selected from carboxy, carboxycarbonylamino and C₃₋₇cycloalkylsulfonylamino-carbonyl;
R⁸ is selected from H, halogen, C₁₋₆alkyl, haloC₁₋₆alkyl and C₁₋₆alkoxy;
R⁹ is selected from H and C₁₋₆alkoxy; and
R¹⁰ is selected from H, halogen, C₁₋₆alkoxy and haloC₁₋₆alkoxy;
or a pharmaceutically acceptable salt thereof.

3. A compound according to claim 2, wherein
R¹ is Cl;
R⁶ is selected from H and methoxy;
R⁷ is selected from carboxy, carboxycarbonylamino and cyclopropylsulfonylamino-carbonyl;
R⁸ is selected from H, Cl, Br, methyl, CF₃ and methoxy;
R⁹ is selected from H and methoxy;
R¹⁰ is selected from H, F, Cl, Br, methoxy, ethoxy, difluoromethoxy and trifluoromethoxy;
G₁ is selected from methyl, ethyl, propyl and cyclobutyl; and
G₂ is selected from methyl and cyclobutyl;
or a pharmaceutically acceptable salt thereof.

4. A compound according to claim 1, or a pharmaceutically acceptable salt thereof, wherein R⁷ is selected from carboxy and C₃₋₇cycloalkylsulfonylamino-carbonyl.

5. A compound according to claim 4, or a pharmaceutically acceptable salt thereof, wherein R⁷ is selected from carboxy and cyclopropylsulfonylaminocarbonyl.

6. A compound according to claim 1, or a pharmaceutically acceptable salt thereof, wherein R⁹ is H.

7. A compound according to claim 1, or a pharmaceutically acceptable salt thereof, wherein R¹⁰ is selected from H, halogen and C₁₋₆alkoxy.

8. A compound according to claim 7, or a pharmaceutically acceptable salt thereof, wherein R¹⁰ is selected from H, F, methoxy and ethoxy.

9. A compound according to claim 1,
wherein:
R¹ is halogen;
R², R³, R⁴, and R⁵ are H;
R⁶ is selected from H and C₁₋₆alkoxy;
R⁷ is selected from carboxy and C₃₋₇cycloalkylsulfonylaminocarbonyl;
R⁸ is selected from H, halogen, C₁₋₆alkyl, haloC₁₋₆alkyl and C₁₋₆alkoxy;
R⁹ is H; and
R¹⁰ is selected from H, halogen and C₁₋₆alkoxy;
or a pharmaceutically acceptable salt thereof.

10. A compound according to claim 9, wherein:
R¹ is Cl;
R⁶ is selected from H and methoxy;
R⁷ is selected from carboxy and cyclopropylsulfonylaminocarbonyl;
R⁸ is selected from H, Cl, Br, methyl, CF₃ and methoxy;
R¹⁰ is selected from H, F, methoxy and ethoxy;
G₁ is selected from methyl, ethyl, propyl and cyclobutyl; and
G₂ is selected from methyl and cyclobutyl;
or a pharmaceutically acceptable salt thereof.

11. A compound according to claim 1, selected from:
cis-3-[2-[2-bromo-5-(8-chloro-4-oxo-chromen-2-yl)-4-methoxy-phenoxy]ethoxy]-cyclobutanecarboxylic acid;
3-[2-bromo-5-(8-chloro-4-oxo-chromen-2-yl)-4-methoxy-phenoxy]propanoic acid;
3-[2-bromo-5-(8-chloro-4-oxo-chromen-2-yl)-4-methoxy-phenoxy]-N-cyclopropylsulfonyl-propanamide;
2-[2-bromo-5-(8-chloro-4-oxo-chromen-2-yl)-4-methoxy-phenoxy]acetic acid;
3-[2-bromo-5-(8-chloro-4-oxo-chromen-2-yl)-4-methoxy-phenoxy]cyclobutanecarboxylic acid;
2-[3-[2-bromo-5-(8-chloro-4-oxo-chromen-2-yl)-4-methoxy-phenoxy]propoxy]acetic acid;
3-[2-chloro-5-(8-chloro-4-oxo-chromen-2-yl)-4-methoxy-phenoxy]cyclobutanecarboxylic acid;
cis-3-[2-[2-chloro-5-(8-chloro-4-oxo-chromen-2-yl)-4-methoxy-phenoxy]ethoxy]-cyclobutanecarboxylic acid;
3-[2-[5-(8-chloro-4-oxo-chromen-2-yl)-4-methoxy-2-methyl-phenoxy]ethoxy]-cyclobutanecarboxylic acid;
3-[5-(8-chloro-4-oxo-chromen-2-yl)-4-methoxy-2-methyl-phenoxy]propanoic acid;
2-[3-[5-(8-chloro-4-oxo-chromen-2-yl)-4-methoxy-2-methyl-phenoxy]propoxy]acetic acid;
3-[5-(8-chloro-4-oxo-chromen-2-yl)-2,4-dimethoxy-phenoxy]cyclobutanecarboxylic acid;
3-[2-[5-(8-chloro-4-oxo-chromen-2-yl)-2,4-dimethoxy-phenoxy]ethoxy]cyclobutane-carboxylic acid;
2-[5-(8-chloro-4-oxo-chromen-2-yl)-2-methyl-phenoxy]acetic acid;
cis-3-[5-(8-chloro-4-oxo-chromen-2-yl)-2-methyl-phenoxy]cyclobutanecarboxylic acid;
trans-3-[5-(8-chloro-4-oxo-chromen-2-yl)-2-methyl-phenoxy]cyclobutanecarboxylic acid;
3-[5-(8-chloro-4-oxo-chromen-2-yl)-2-methoxy-phenoxy]cyclobutanecarboxylic acid;
3-[2-[5-(8-chloro-4-oxo-chromen-2-yl)-2-methoxy-phenoxy]ethoxy]cyclobutanecarboxylic acid;
3-[5-(8-chloro-4-oxo-chromen-2-yl)-2-methoxy-phenoxy]propanoic acid;
cis-3-[5-(8-chloro-4-oxo-chromen-2-yl)-2-(trifluoromethyl)phenoxy]cyclobutanecarboxylic acid;
trans-3-[5-(8-chloro-4-oxo-chromen-2-yl)-2-(trifluoromethyl)phenoxy]cyclobutanecarboxylic acid;
2-[5-(8-chloro-4-oxo-chromen-2-yl)-2-(trifluoromethyl)phenoxy]acetic acid;
cis-3-[5-(8-chloro-4-oxo-chromen-2-yl)-2,3-dimethoxy-phenoxy]cyclobutanecarboxylic acid;
trans-3-[2-[5-(8-chloro-4-oxo-chromen-2-yl)-2,3-dimethoxy-phenoxy]ethoxy]cyclobutane-carboxylic acid;
2-[5-(8-chloro-4-oxo-chromen-2-yl)-2,3-dimethoxy-phenoxy]acetic acid;
trans-3-[4-bromo-5-(8-chloro-4-oxo-chromen-2-yl)-2-methoxy-phenoxy]cyclobutane-carboxylic acid;
trans-3-[2-[4-bromo-5-(8-chloro-4-oxo-chromen-2-yl)-2-methoxy-phenoxy]ethoxy]-cyclobutanecarboxylic acid;
3-[3-(8-chloro-4-oxo-chromen-2-yl)-2-methoxy-phenoxy]cyclobutanecarboxylic acid;
cis-3-[2-[3-(8-chloro-4-oxo-chromen-2-yl)-2-methoxy-phenoxy]ethoxy]cyclobutane-carboxylic acid;
3-[3-(8-chloro-4-oxo-chromen-2-yl)-4-methoxy-phenoxy]cyclobutanecarboxylic acid;
cis-3-[2-[3-(8-chloro-4-oxo-chromen-2-yl)-4-methoxy-phenoxy]ethoxy]cyclobutane-carboxylic acid;
2-[3-(8-chloro-4-oxo-chromen-2-yl)-4-methoxy-phenoxy]acetic acid;
3-[3-(8-chloro-4-oxo-chromen-2-yl)-4-methoxy-phenoxy]propanoic acid;
cis-3-[2-[6-bromo-3-(8-chloro-4-oxo-chromen-2-yl)-2-methoxy-phenoxy]ethoxy]-cyclobutanecarboxylic acid;
3-[6-bromo-3-(8-chloro-4-oxo-chromen-2-yl)-2-methoxy-phenoxy]cyclobutanecarboxylic acid;
3-[3-(8-chloro-4-oxo-chromen-2-yl)-2-methoxy-6-methyl-phenoxy]cyclobutanecarboxylic acid;
2-[6-chloro-3-(8-chloro-4-oxo-chromen-2-yl)-2-methoxy-phenoxy]acetic acid;
2-[3-(8-chloro-4-oxo-chromen-2-yl)-2,6-dimethoxy-phenoxy]acetic acid;
3-[2-bromo-5-(8-chloro-4-oxo-chromen-2-yl)-4-ethoxy-phenoxy]propanoic acid;
3-[2-bromo-5-(8-chloro-4-oxo-chromen-2-yl)-4-(difluoromethoxy)phenoxy]propanoic acid;
3-[2-bromo-5-(8-chloro-4-oxo-chromen-2-yl)-4-fluoro-phenoxy]propanoic acid;
3-[2-bromo-5-(8-chloro-4-oxo-chromen-2-yl)-4-(trifluoromethoxy)phenoxy]propanoic acid;
3-[2-bromo-4-chloro-5-(8-chloro-4-oxo-chromen-2-yl)phenoxy]propanoic acid;
2-[3-[6-bromo-3-(8-chloro-4-oxo-chromen-2-yl)-2-methoxy-phenoxy]propylamino]-2-oxo-acetic acid;
2-[2-[2-bromo-5-(8-chloro-4-oxo-chromen-2-yl)-4-methoxy-phenoxy]ethylamino]-2-oxo-acetic acid; and
2-[3-[2-bromo-5-(8-chloro-4-oxo-chromen-2-yl)-4-methoxy-phenoxy]propylamino]-2-oxo-acetic acid;
or a pharmaceutically acceptable salt thereof.

12. A compound according to claim 1, selected from:
3-[2-bromo-5-(8-chloro-4-oxo-chromen-2-yl)-4-methoxy-phenoxy]propanoic acid;
3-[2-bromo-5-(8-chloro-4-oxo-chromen-2-yl)-4-methoxy-phenoxy]-N-cyclopropylsulfonyl-propanamide;
2-[2-bromo-5-(8-chloro-4-oxo-chromen-2-yl)-4-methoxy-phenoxy]acetic acid;
2-[3-[2-bromo-5-(8-chloro-4-oxo-chromen-2-yl)-4-methoxy-phenoxy]propoxy]acetic acid;
cis-3-[2-[2-chloro-5-(8-chloro-4-oxo-chromen-2-yl)-4-methoxy-phenoxy]ethoxy]-cyclobutanecarboxylic acid;
3-[5-(8-chloro-4-oxo-chromen-2-yl)-2,4-dimethoxy-phenoxy]cyclobutanecarboxylic acid;
3-[2-[5-(8-chloro-4-oxo-chromen-2-yl)-2,4-dimethoxy-phenoxy]ethoxy]cyclobutane-carboxylic acid;
cis-3-[5-(8-chloro-4-oxo-chromen-2-yl)-2-methyl-phenoxy]cyclobutanecarboxylic acid;
2-[5-(8-chloro-4-oxo-chromen-2-yl)-2-(trifluoromethyl)phenoxy]acetic acid;
3-[3-(8-chloro-4-oxo-chromen-2-yl)-2-methoxy-phenoxy]cyclobutanecarboxylic acid;
cis-3-[2-[6-bromo-3-(8-chloro-4-oxo-chromen-2-yl)-2-methoxy-phenoxy]ethoxy]-cyclobutanecarboxylic acid;
3-[6-bromo-3-(8-chloro-4-oxo-chromen-2-yl)-2-methoxy-phenoxy]cyclobutanecarboxylic acid;
2-[3-(8-chloro-4-oxo-chromen-2-yl)-2,6-dimethoxy-phenoxy]acetic acid;
3-[2-bromo-5-(8-chloro-4-oxo-chromen-2-yl)-4-ethoxy-phenoxy]propanoic acid; and
3-[2-bromo-5-(8-chloro-4-oxo-chromen-2-yl)-4-fluoro-phenoxy]propanoic acid;
or a pharmaceutically acceptable salt thereof.

13. A process for preparing a compound according to claim 1, the process comprising at least one of the following steps:
(a) hydrolyzing a compound of formula (III-6), 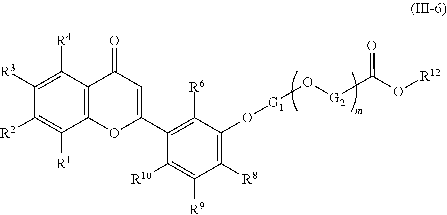
in the presence of a base or an acid;
(b) hydrolyzing a compound of formula (IV-1), 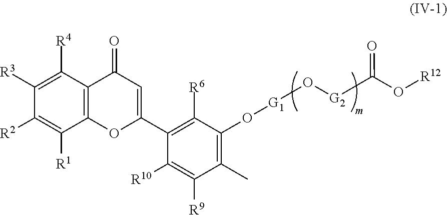
in the presence of a base or an acid;
(c) reacting a compound of formula (III-4), 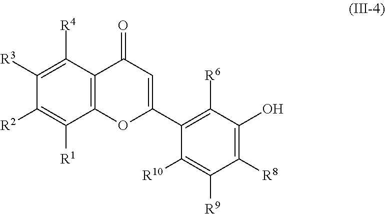
with oxetan-2-one in the presence of a base;
(d) oxidizing a compound of formula (V-1), 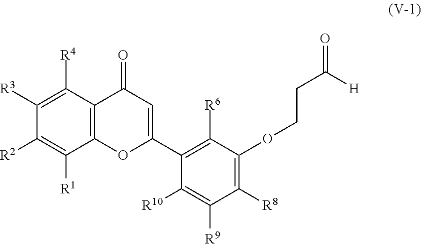
with NaClO₂;
(e) reacting a compound of formula (III-7), 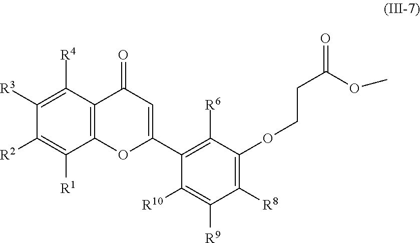
with sulfonamide in the presence of a Lewis acid;
and
(f) hydrolyzing a compound of formula (VI-3), 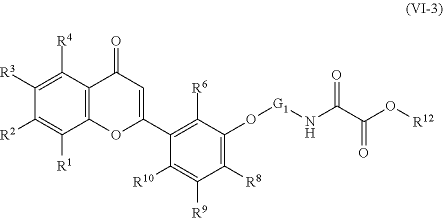
in the presence of a base;
wherein R¹² is C₁₋₆alkyl.

14. (canceled)

15. A pharmaceutical composition comprising a compound in accordance with claim 1 and a therapeutically inert carrier.

16. (canceled)

17. (canceled)

18. (canceled)

19. (canceled)

20. (canceled)

21. (canceled)

22. (canceled)

23. A compound when manufactured according to the process of claim 13.

24. A method for the treatment or prophylaxis of HBV infection, which method comprises administering an effective amount of a compound as defined in claim 1 to a patient in need thereof.

25. A pharmaceutical composition comprising a compound in accordance with claim 1 and a therapeutically inert carrier.

26. A pharmaceutical composition comprising a compound in accordance with claim 11 and a therapeutically inert carrier.

27. A method of inhibiting a target selected from cccDNA, HbeAg, HbsAg, and HBV DNA, the method comprising administering to a patient an effective amount of a compound of claim 1.

28. A method of inhibiting a target selected from cccDNA, HbeAg, HbsAg, and HBV DNA, the method comprising administering to a patient an effective amount of a compound of claim 11.

29. A method for the treatment or prophylaxis of HBV infection, which method comprises administering an effective amount of a compound as defined in claim 11 to a patient in need thereof.
